(19) United States
(12) Patent Application Publication
(10) Pub. No.: US 2024/0318204 A1
(43) Pub. Date: Sep. 26, 2024

(54) STRATEGIES TO DEVELOP GENOME EDITING SPHERICAL NUCLEIC ACIDS (SNAS)

(71) Applicant: NORTHWESTERN UNIVERSITY, Evanston, IL (US)

(72) Inventors: Chad A. Mirkin, Wilmette, IL (US); Isaac Larkin, Chicago, IL (US); Chi Huang, Evanston, IL (US)

(21) Appl. No.: 18/279,034

(22) PCT Filed: Feb. 25, 2022
(86) PCT No.: PCT/US22/17984

§ 371 Date: Aug. 25, 2023

Related U.S. Application Data
(60) Provisional application No. 63/290,522, filed on Dec. 16, 2021.
(60) Provisional application No. 63/154,530, filed on Feb. 26, 2021.
(60) Provisional application No. 63/273,086, filed on Oct. 28, 2021.

(51) Int. Cl. C12N 15/88; C12N 9/22; C12N 15/11

(52) U.S. Cl. CPC C12N 15/88; C12N 9/22; C12N 15/11; C07K 2319/09; C12N 2310/20; C12N 2320/32; C12N 2800/80

(57) ABSTRACT

Spherical nucleic acids (SNAs) are an attractive platform for therapeutic delivery due to their chemically tunable structures, biocompatibility, and ability to rapidly enter cells without transfection reagents. The present disclosure provides SNAs and strategies for delivering gene editing proteins into cells. The delivered gene editing proteins remain enzymatically active and rapidly enter mammalian cells.

### CROSS-REFERENCE TO RELATED APPLICATIONS

This application claims the priority benefit under 35 U.S.C. § 119(e) of U.S. Provisional Patent Application No. 63/154,530, filed Feb. 26, 2021, U.S. Provisional Patent Application No. 63/273,086, filed Oct. 28, 2021 and U.S. Provisional Patent Application No. 63/290,522, filed Dec. 16, 2021, which are incorporated herein by reference in their entirety.

### STATEMENT OF GOVERNMENT INTEREST

This invention was made with government support under grant number DJF-15-1200-K-0001730 awarded by the Federal Bureau of Investigation (FBI). The government has certain rights in the invention.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 2021-043R_Seqlisting.txt; Size: 61,129 bytes; Created: Feb. 25, 2022.

### BACKGROUND

Genome editing refers to the removal or the insertion of a specific DNA sequence. Among the members of genome editing proteins, the CRISPR/Cas9 (clustered regularly interspaced short palindromic repeat, and CRISPR-associated protein 9) protein has been exploited as efficient genome editing tools to edit and modulate genome for clinic translation because of its specificity and versatility [P. Horvath, R. Barrangou, Science 2010, 327, 167-170]. While considerable achievements of Cas9 enzyme have been made, reduced off-target effects and efficient and direct transduction of Cas9-single guide RNA (sgRNA) complexes is still highly desirable [L. Y. Chou, K. Ming, W. C. Chan, Chem. Soc. Rev. 2011, 40, 233-245; V. Biju, Chem. Soc. Rev. 2014, 43, 744-764; Y. Lu, A. A. Aimetti, R. Langer, Z. Gu, Nat. Rev. Mater. 2017, 2, 16075].

Rapidly programmable nucleases such as Clustered Regularly Interspaced Short Palindromic Repeats (CRISPR)/CRISPR associated (Cas) protein and Transcription Activator-Like Effector Nucleases (TALENs) have the potential to treat a wide range of genetic diseases [Gupta et al., J Clin Invest. 124(10): 4154-4161 (2014); Hsu et al., Cell 157(6): 1262-1278 (2014)], but efficient delivery into mammalian cells remains a challenge.

### SUMMARY

To attempt to address current limitations with genome editing including off-target effects and efficient transduction of gene editing proteins, various nonviral delivery systems, such as cationic liposomes, cationic polymers, and inorganic nanoparticles have been designed and employed for stabilizing and enhancing delivery of Cas9-sgRNA complexes [Y. Fu, J. A. Foden, C. Khayter, M. L. Maeder, D. Reyon, J. K. Joung, J. D. Sander, Nat. Biotechnol. 2013, 31, 822-826; J. G. Doench, N. Fusi, M. Sullender, M. Hegde, E. W. Vaimberg, K. F. Donovan, I. Smith, Z. Tothova, C. Wilen, R. Orchard, H. W. Virgin, J. Listgarten, D. E. Root, Nat. Biotechnol. 2016, 34, 184-191; B. P. Kleinstiver, V. Pattanayak, M. S. Prew, S. Q. Tsai, N. T. Nguyen, Z. Zheng, J. K. Joung, Nature 2016, 529, 490-495; I. M. Slaymaker, L. Gao, B. Zetsche, D. A. Scott, W. X. Yan, F. Zhang, Science 2016, 351, 84-88]. However, the complicated designs of these carriers, releasing efficiency, and potential toxic and immunogenic side effects impede their rapid clinical adoption. Viral systems have been used as a first resort to transduce cells in vivo. These systems suffer from problems related to packaging constraints, immunogenicity, and longevity of Cas expression, which favors off-target events. Viral vectors are as such not the best choice for direct in vivo delivery of CRISPR/Cas. The present disclosure is directed to spherical nucleic acids, which comprise a shell of oligonucleotides attached to a nanoparticle core, and their use in the delivery of gene editing proteins.

Accordingly, in some aspects the disclosure provides a protein-core spherical nucleic acid (ProSNA) comprising (a) a protein core that comprises a gene editing protein; and (b) a shell of oligonucleotides attached to the protein core. In some embodiments, each oligonucleotide in the shell of oligonucleotides is covalently attached to the protein core. In some embodiments, each oligonucleotide in the shell of oligonucleotides is attached to the protein core through a linker. In further embodiments, the linker is a cleavable linker, a non-cleavable linker, a traceless linker, or a combination thereof. In still further embodiments, the linker is a carbamate alkylene dithiolate linker. In some embodiments, at least one oligonucleotide in the shell of oligonucleotides comprises protein-core-NH—C(O)—O—C₂₋₅alkylene-S—S—C₂₋₇alkylene-Oligonucleotide, or protein-core-NH—C(O)—O—CH₂Ar—S—S—C₂₋₇alkylene-Oligonucleotide, and Ar comprises a meta-or para-substituted phenyl. In some embodiments, at least one oligonucleotide in the shell of oligonucleotides comprises protein-core-NH—C(O)—O—C(ZA)(ZB)C₁₋₄alkylene-C(XA)(XB)—S—S—C(YA)(YB)C₁₋₆alkylene-Oligonucleotide, and ZA, ZB, XA, XB, YA, and YB are each independently H, Me, Et, or iPr. In some embodiments, at least one oligonucleotide in the shell of oligonucleotides comprises protein-core-NH—C(O)—O—C(XA)(XB)—Ar—S—S—C(YA)(YB)C₂₋₆alkylene-Oligonucleotide, and XA, XB, YA, and YB are each independently H, Me, Et, or iPr. In some embodiments, the linker is an amide alkylene dithiolate linker. In further embodiments, at least one oligonucleotide in the shell of oligonucleotides comprises protein-core-NH—C(O)—C₂₋₅alkylene-S—S—C₂₋₇alkylene-Oligonucleotide. In some embodiments, at least one oligonucleotide in the shell of oligonucleotides comprises protein-core-NH—C(O)—C₁₋₄alkylene-C(XA)(XB)—S—S—C(YA)(YB)C₁₋₆alkylene-Oligonucleotide, and XA, XB, YA and YB are each independently H, Me, Et, or iPr. In some embodiments, the linker is an amide alkylene thioether linker. In further embodiments, at least one oligonucleotide in the shell of oligonucleotides comprises protein-core-NH—C(O)—C₂₋₄alkylene-N-succinimidyl-S—C₂₋₆alkylene-Oligonucleotide.

In some aspects, the disclosure provides a spherical nucleic acid (SNA) comprising (a) a nanoparticle core; (b) a shell of oligonucleotides attached to the external surface of the nanoparticle core; and (c) a gene editing protein. In some embodiments, the nanoparticle core is a liposomal core or a lipid nanoparticle core. In further embodiments, the lipid nanoparticle core comprises an ionizable lipid, a phospholipid, a sterol, and a lipid-polyethylene glycol (lipid-PEG) conjugate. In some embodiments, each oligonucleotide in the shell of oligonucleotides is covalently attached to the exterior of the lipid nanoparticle core through the lipid-PEG conjugate. In some embodiments, the gene editing protein is encapsulated in the lipid nanoparticle core. In some embodiments, a ProSNA of the disclosure is encapsulated in the lipid nanoparticle core. In some embodiments, a ribonucleoprotein (RNP) complex is encapsulated in the lipid nanoparticle core, the RNP comprising the gene editing protein, clustered regularly interspaced short palindromic repeat (CRISPR) RNA (crRNA), and trans-activating crRNA (tracrRNA). In some embodiments, the liposomal core comprises a plurality of lipid groups. In some embodiments, the gene editing protein is encapsulated in the liposomal core. In some embodiments, a ProSNA of the disclosure is encapsulated in the liposomal nanoparticle core. In some embodiments, a ribonucleoprotein (RNP) complex is encapsulated in the lipid nanoparticle core, the RNP comprising the gene editing protein, CRISPR RNA (crRNA), and trans-activating crRNA (tracrRNA). In some embodiments, the plurality of lipid groups comprises a lipid selected from the group consisting of the phosphatidylcholine, phosphatidylglycerol, and phosphatidylethanolamine families of lipids. In some embodiments, the lipid is selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE). In some embodiments, at least one oligonucleotide in the shell of oligonucleotides is attached to the exterior of the liposomal or lipid nanoparticle core through a lipid anchor group. In some embodiments, the lipid anchor group is attached to the 5′ end or the 3′ end of the at least one oligonucleotide. In further embodiments, the lipid anchor group is tocopherol or cholesterol. In some embodiments, the gene editing protein is a CRISPR-associated protein (Cas). In further embodiments, the Cas is Cas9, Cas12, Cas13, or a combination thereof. In some embodiments, at least one oligonucleotide in the shell of oligonucleotides is modified on its 5′ end and/or 3′ end with dibenzocyclooctyl (DBCO). In some embodiments, the shell of oligonucleotides comprises single-stranded DNA, double-stranded DNA, single-stranded RNA, double-stranded RNA, or a combination thereof. In some embodiments, at least one oligonucleotide in the shell of oligonucleotides is a modified oligonucleotide. In some embodiments, the shell of oligonucleotides comprises about 2 to about 100 oligonucleotides. In further embodiments, the shell of oligonucleotides comprises about 10 to about 80 oligonucleotides. In some embodiments, the shell of oligonucleotides comprises about 5 to about 50 oligonucleotides. In further embodiments, the shell of oligonucleotides comprises about 5 to about 20 oligonucleotides. In still further embodiments, the shell of oligonucleotides comprises about 14 oligonucleotides. In some embodiments, the shell of oligonucleotides comprises about 15 oligonucleotides. In some embodiments, each oligonucleotide in the shell of oligonucleotides is about 5 to about 100 nucleotides in length. In further embodiments, each oligonucleotide in the shell of oligonucleotides is about 10 to about 50 nucleotides in length. In some embodiments, one or more oligonucleotides in the shell of oligonucleotides comprises a (GGX)ₙ nucleotide sequence, wherein n is 2-20 and X is a nucleobase (A, C, T, G, or U). In some embodiments, the (GGX)ₙ nucleotide sequence is on the 5′ end of the one or more oligonucleotides. In some embodiments, the (GGX)ₙ nucleotide sequence is on the 3′ end of the one or more oligonucleotides. In some embodiments, one or more oligonucleotides in the shell of oligonucleotides comprises a (GGT)ₙ nucleotide sequence, wherein n is 2-20. In some embodiments, the (GGT)ₙ nucleotide sequence is on the 5′ end of the one or more oligonucleotides. In some embodiments, the (GGT)ₙ nucleotide sequence is on the 3′ end of the one or more oligonucleotides. In some embodiments, diameter of the ProSNA or SNA is about 1 nanometer (nm) to about 500 nm. In some embodiments, diameter of the SNA is less than or equal to about 50 nanometers. In some embodiments, at least one oligonucleotide in the shell of oligonucleotides is a targeting oligonucleotide. In some embodiments, the shell of oligonucleotides comprises an inhibitory oligonucleotide, an immunostimulatory oligonucleotide, a gene editor substrate DNA or RNA, or a combination thereof. In further embodiments, the inhibitory oligonucleotide is an antisense oligonucleotide, small interfering RNA (siRNA), an aptamer, a short hairpin RNA (shRNA), a DNAzyme, or an aptazyme. In some embodiments, the immunostimulatory oligonucleotide is a CpG-motif containing oligonucleotide, a double-stranded DNA oligonucleotide, or a single-stranded RNA oligonucleotide. In further embodiments, each of the immunostimulatory oligonucleotides is a toll-like receptor (TLR) agonist. In still further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

In some aspects, the disclosure provides a composition comprising a plurality of protein-core spherical nucleic acids (ProSNAs) as described herein. In some embodiments, the composition further comprises a guide RNA. In some embodiments, at least two of the ProSNAs comprise a different protein core.

In some aspects, the disclosure provides a composition comprising a plurality of spherical nucleic acids (SNAs) of the disclosure. In some embodiments, at least two of the SNAs comprise a different nanoparticle core.

In some aspects, the disclosure provides a method of delivering a gene editing protein to a cell comprising contacting the cell with a ProSNA of the disclosure.

In some aspects, the disclosure provides a method of delivering a gene editing protein to a cell comprising contacting the cell with a composition of the disclosure.

In some aspects, the disclosure provides a method of delivering a gene editing protein to a cell comprising contacting the cell with a SNA of the disclosure.

In some aspects, the disclosure provides a method of delivering a gene editing protein to a cell comprising contacting the cell with a composition of the disclosure.

In some aspects, the disclosure provides a method of treating, ameliorating, and/or preventing a disorder in a subject comprising administering to the subject an effective amount of (i) a ProSNA of the disclosure, (ii) a SNA of the disclosure, (iii) a composition of the disclosure, or (iv) a combination thereof. In some embodiments, the disorder is cancer, an infectious disease, an autoimmune disease, a neurodegenerative disease, an inherited disease, cardiovascular disease, or a combination thereof.

In some aspects, the disclosure provides a fused protein comprising the following. arranged from N-terminus to C-terminus as follows: (i) one or more GALA peptides; (ii) a gene editing protein, and (iii) a nuclear localization signal (NLS). In some embodiments, the one or more GALA peptides comprises three successive GALA peptides. In various embodiments, each of the one or more GALA peptides comprises or consists of an amino acid sequence that is at least 90% identical to the amino acid sequence as set out in SEQ ID NO: 22. In some embodiments, the one or more GALA peptides comprises or consists of the amino acid sequence as set out in SEQ ID NO: 26. In some embodiments, the gene editing protein is a CRISPR-associated protein (Cas). In further embodiments, the Cas is Cas9, Cas12, Cas13, or a combination thereof. In some embodiments, the Cas9 comprises or consists of an amino acid sequence that is at least 95% identical to the amino acid sequence as set out in SEQ ID NO: 1 or SEQ ID NO: 25. In some embodiments, the Cas12 comprises or consists of an amino acid sequence that is at least 95% identical to the amino acid sequence as set out in SEQ ID NO: 27. In some embodiments, the Cas13 comprises or consists of an amino acid sequence that is at least 95% identical to the amino acid sequence as set out in SEQ ID NO: 29. In various embodiments, the NLS comprises or consists of an amino acid sequence that is at least 95% identical to the amino acid sequence as set out in SEQ ID NO: 23 or SEQ ID NO: 28.

In some aspects, the disclosure provides a composition comprising a fused protein of the disclosure and a pharmaceutically acceptable carrier.

In further aspects, the disclosure provides a ProSNA as described herein, wherein the gene editing protein is a fused protein of the disclosure.

In some aspects, the disclosure provides a SNA as described herein, wherein the gene editing protein is a fused protein of the disclosure.

In further aspects, the disclosure provides a method of delivering a gene editing protein to a cell comprising contacting the cell with a fused protein as described herein.

In some aspects, the disclosure provides a method of delivering a gene editing protein to a cell comprising contacting the cell with a composition of the disclosure comprising a fused protein.

In further aspects, the disclosure provides a method of treating, ameliorating, and/or preventing a disorder in a subject comprising administering to the subject an effective amount of (i) a fused protein of the disclosure, (ii) a composition of the disclosure comprising a fused protein, or (iii) a combination thereof. In some embodiments, the disorder is cancer, an infectious disease, an autoimmune disease, a neurodegenerative disease, an inherited disease, cardiovascular disease, or a combination thereof.

### BRIEF DESCRIPTION OF THE FIGURES

FIG. 1 is a schematic of the synthesis of CRISPR-SNAs. Concentrated Cas9 RNPs are encapsulated in liposomes, most unencapsulated RNPs are removed via SEC, liposomes were extruded to reduce polydispersity, DBCO-DNA is added to functionalize liposomes with DNA, liposomes are incubated with proteinase K to digest remaining unencapsulated Cas9, and finally digested Cas9 is removed via SEC.

FIG. 2 shows: (A) DLS of CRISPR SNAs after DNA functionalization and cleaning. (B) Standard curve of Cy3-DNA fluorescence, with SNA sample (diluted by half). (C) ICP-OES quantification of phosphorus (and therefore phospholipid) concentration in CRISPR SNA sample, including standard curve (blue), SNA sample (red), and SNA sample after correcting for the concentration of DNA obtained in B. SNA concentration is calculated using equation 1. (D) Standard curve of Alexa647-RNP fluorescence, with SNA sample (blue) plotted with a linear fit.

FIG. 3 shows that RNPs remain active throughout SNA synthesis procedure. (A) Schematic of the in vitro Cas9 activity test. (B) Activity tests of fresh Cas9 RNP (B1), Cas9 RNPs that were modified with Alexa dye (B2), then concentrated with Amicon 10K filters (B3), then subjected to 7 cycles of freeze/thaw/sonication (B4), then run through Sepharose 6b SEC columns (B5), then extruded 3× through 0.2 μM and 0.1 μM PES membranes (B6).

FIG. 4 demonstrates that CRISPR-SNAs protect active RNPs from protease, indicating encapsulation. (A) Size exclusion fractions collected from a Superdex 200 column after incubating proteinase K with a mixture of empty SNAs and Alexa-RNPs (top) or CRISPR SNAs with encapsulated Alexa-RNPs (bottom). Cy3 (DNA) fluorescence is shown in red, Alexa647 (Cas9) fluorescence in blue, and co-localization of Cy3 and Cas9 fluorescence in pink. (B) In vitro Cas9 activity tests were run with no Cas9 (1); fresh Cas9 without proteinase K (2) and with proteinase K (3); Alexa-modified Cas9 without proteinase K (4) and with proteinase K (5); CRISPR liposomes without proteinase K (6), with proteinase K (7); and with proteinase K added after disrupting liposomes with Tween 20 (8); and finally, CRISPR SNAs without proteinase K (9), with proteinase K (10), and with proteinase K added after disrupting liposomes with Tween 20 (11).

FIG. 5 shows that CRISPR-SNAs are actively taken up into mammalian cells. After incubating 5 picomole-equivalents of Alexa RNP of each sample with C166-GFP cells for 16 hours, Alexa 647 fluorescence measured on the allophycocyanin (APC) excitation and emission filter. Histogram of Alexa-RNP fluorescence for untreated cells (red, overlaps with Empty liposomal spherical nucleic acid (LSNA), empty Cy3-modified LSNA (bright green), RNPs encapsulated in liposomes (orange), Alexa-RNPs transfected with RNAiMax, and finally CRISPR SNAs (dark green).

FIG. 6 shows the structure characterization of ProSNA (dashed red traces) Cas9. (A) TEM characterization of Cas9 SNA. (B) and (C) Denaturing gel electrophoresis and Zeta potentials of unmodified Cas9, Cas9 AF647, Cas9 azide and Cas9 SNA. (D) UV-vis absorbance spectra used to quantitate the functionalization of Cas9 with AlexaFluor 647 and DNA.

FIG. 7 shows results from cell experiments demonstrating the biocompatibility and cellular uptake. (a) cell viability of HaCat, HEK 293T, hMSCs, or Raw 264.7 cells treated with Cas9 SNA for 48 hours; (b) Cellular uptake of Cas9 (white) and Cas9 SNA (black), as determined by flow cytometry.

FIG. 8 depicts HEK293T/EGFP cell genome editing of Cas9 SNA. Surveyor assays of (a) DNase I hypersensitive site, (b) GRIN2B and (c) EGFP. d) Flow cytometry of HEK293T/EGFP cells treated with Cas9 SNA.

FIG. 9 shows a schematic design of engineering GeoCas9 was fused with GALA endosome peptides at N-terminus.

FIG. 10 shows quantitative molar extinction coefficients of GeoCas9 at (a) 260 nm and (b) 280 nm. The molar extinction coefficients were determined by Pierce bicinchoninic acid assay and used to quantitate the concentration of GeoCas9 and Cas9 SNAs.

FIG. 11 depicts the structure of Alexa Fluor™ 647 NHS Ester (AF647) used to prepare Cas9-AF647.

FIG. 12 shows UV-Vis spectrum of AF-647 fluorophore modified Cas9. Spectroscopy was determined at ambient temperature on a Cary5000 spectrophotometer. Protein and AF647 concentrations were calculated from the absorbance at 650 nm and 280 nm, respectively. The AF647 fluorophore was used to calculate the concentration of protein after DNA modification and track the protein uptake both in the flow cytometry and confocal imaging experiments. Inset: Calculations details of fluorophores per Cas9.

FIG. 13 shows the structure of NHS-PEG₄-Azide linker used to prepare azide terminated Cas9 (Cas9-AF647-azide).

FIG. 14 shows MALDI-MS spectra of unmodified Cas9-AF647 (blue) and Cas9-AF647-azide (red). To calculate the number of NHS-PEG₄-azides per protein, MALDI-MS was used to determine the mass difference between an unmodified and azide modified protein. Each linker conjugation leads to an mass increase of 275 m/z.

FIG. 15 shows the determination of the number of DNA strands on Cas9 ProSNAs with UV-Vis spectrum. Spectrum were determined on a Cary5000 spectrophotometer. Protein and DNA concentrations were calculated from the absorbance at 650 nm and 260 nm, respectively. Inset: Calculations details of DNA per Cas9.

FIG. 16 shows FPLC size-exclusion chromatogram (SEC) analysis of (a) Cas9 SNAs (b) and Cas9-AF647-azide. Solid lines correspond to extinction at 650 nm, and dashed lines to 260 nm. All samples were ran on an SEC650 column (Bio-Rad) at a flow rate of 1 mL/min at 4° C.

FIG. 17 shows SDS-PAGE gel biostability analysis of (a) Cas9 and (b) Cas9 ProSNA incubated with trypsin (protease), showing that while Cas9 degraded over a time course of 1 hour (as evidenced by the disappearance of Cas9 protein bands), Cas9 ProSNA remained.

FIG. 18 shows cell viability measurement with live and dead analysis of Cas9 ProSNAs in HaCat cells. Live cells were stained with Calcium AM and dead cells were stained with propidium iodide (PI). No significant cell toxicity was observed after treatment of Cas9 Protein, as determined by fluorescence microscopy. Scale bars: 300 μm.

FIG. 19 shows flow histograms depicting cellular uptake of AF647 modified Cas9 ProSNAs and native Cas9 in HaCat cells. Flow cytometry was used to measure the uptake of Cas9 ProSNA or native protein in HaCat cells after 4 hour treatments with 20 nM protein.

FIG. 20 shows nuclear import efficiency results of HaCat cells treated with Cas9-AF647 and Cas9 ProSNAs at different time points, showing enhanced nucleus import of Cas9 ProSNAs.

FIG. 21 depicts the SURVEYOR assay for detection of double strand break-induced micro insertions and deletions. Schematic of the SURVEYOR assay used to determine Cas9-mediated cleavage efficiency. First, genomic PCR (gPCR) is used to amplify the Cas9 target region from a heterogeneous population of modified and unmodified cells, and the gPCR products are rehybridized slowly to generate heteroduplexes. The reannealed heteroduplexes are cleaved by T7EI nuclease, whereas homoduplexes are left intact. Cas9-mediated cleavage efficiency (% indel) is calculated based on the fraction of cleaved DNA.

FIG. 22 shows genome editing analysis. Flow cytometry histogram results of HEK293T/EGFP cells treated with Cas9 protein, or Cas9 ProSNAs.

FIG. 23 shows surface reactive lysine chemistry enables DNA conjugation to Cas9. FIG. 24 shows the structure of Cas9 was retained after DNA functionalization.

FIG. 25 shows that the Cas9 ProSNAs demonstrated enhanced stability against protease degradation.

FIG. 26 shows that cells incubated with Cas9 ProSNAs demonstrate high cellular viability in multiple cell types, including HaCaT, HEK293T, hMSC, and RAW 264.7 cells.

FIG. 27 shows enhanced cellular uptake by cells treated with Cas9 ProSNAs as observed by AlexaFluor 647 fluorescence.

FIG. 28 depicts barriers to cellular delivery of gene editing proteins and advantages provided by SNAs comprising a protein (e.g., a fused protein) of the disclosure.

FIG. 29 shows that Cas9 SNAs fused with GALA and NLS demonstrated significant endosomal escape and nuclear import efficiency.

FIG. 30 shows Cas9 ProSNAs achieved high gene editing efficiency for both insertion and deletion compared to the control Cas9 protein in HaCaT and hMSC cells.

FIG. 31 demonstrates the editing efficiency of Cas9 ProSNAs in macrophage-like RAW264.7 cells. Cas9 ProSNAs demonstrated increase gene editing activity compared to the control Cas9 protein and commercial transfection agent.

FIG. 32 demonstrates the gene silencing activity of Cas9 ProSNAs in HEK293T cells. Cas9 ProSNAs demonstrated increased knockdown of GFP compared to the control Cas9 protein.

### DETAILED DESCRIPTION

Spherical Nucleic Acids (SNAs) are a class of nanoparticles functionalized with a dense layer of oligonucleotides surrounding an exchangeable nanoparticle core. This nucleic acid shell imparts several functionalities: the oligonucleotide coating forms a highly concentrated salt cloud that decreases endonuclease activity on the nanoparticle surface, and interacts with cell surface proteins, resulting in high cellular uptake in virtually all cell lines. The combination of these unique characteristics allows SNAs to behave as easily tailorable, single-entity agents.

### Terminology

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

As used in this specification and the appended claims, the articles “a” and “an” refer to one or to more than one (for example, to at least one) of the grammatical object of the article.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values. The terms “polynucleotide” and “oligonucleotide” are interchangeable as used herein.

A “linker” as used herein is a moiety that joins an oligonucleotide to a protein core of a protein-core spherical nucleic acid (ProSNA), as described herein. In any of the aspects or embodiments of the disclosure, a linker is a cleavable linker, a non-cleavable linker, a traceless linker, or a combination thereof.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting a therapeutic agent to a subject in need of treatment with such an agent. Such modes include, but are not limited to, oral, topical, intravenous, intraarterial, intraperitoneal, intramuscular, intratumoral, intradermal, intranasal, and subcutaneous administration.

As used herein, “treating” and “treatment” refers to any reduction in the severity and/or onset of symptoms associated with an abnormal scar. Accordingly, “treating” and “treatment” includes therapeutic and prophylactic measures. One of ordinary skill in the art will appreciate that any degree of protection from, or amelioration of, an abnormal scar is beneficial to a subject, such as a human patient. The quality of life of a patient is improved by reducing to any degree the severity of symptoms in a subject and/or delaying the appearance of symptoms.

As used herein, a “targeting oligonucleotide” is an oligonucleotide that directs a SNA to a particular tissue and/or to a particular cell type. In some embodiments, a targeting oligonucleotide is an aptamer. Thus, in some embodiments, a SNA of the disclosure comprises an aptamer attached to the exterior of the nanoparticle core, wherein the aptamer is designed to bind one or more receptors on the surface of a certain cell type.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. Single-stranded RNA sequences can be recognized by toll-like receptors 8 and 9, double-stranded RNA sequences can be recognized by toll-like receptor 3, and double-stranded DNA can be recognized by toll-like receptor 3 and cyclic GMP-AMP synthase (cGAS).

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), DNA or RNA inhibitors of innate immune receptors, an aptamer, a DNAzyme, or an aptazyme.

All references, patents, and patent applications disclosed herein are incorporated by reference with respect to the subject matter for which each is cited, which in some cases may encompass the entirety of the document.

### GENE EDITING PROTEINS

SNAs of the disclosure comprise one or more gene editing proteins. Gene editing proteins contemplated by the disclosure include, without limitation, a transcription activator-like effector-based nucleases (TALEN), a meganuclease, a nuclease, a zinc finger nuclease (ZFN), a CRISPR-associated protein, CRISPR/Cas9, Cas9, xCas9, Cas12a (Cpf1), Cas13, Cas13a, Cas14, CasX, CasY, a Class 1 Cas protein, a Class 2 Cas protein, MAD7, or a combination thereof. In any aspects or embodiments of the disclosure, genome editing is used to inhibit or reduce production of a target gene. In certain embodiments, the reduction of gene expression and subsequently of biological active protein expression can be achieved by insertion/deletion of nucleotides via non-homologous end joining (NHEJ) or the insertion of appropriate donor cassettes via homology directed repair (HDR) that lead to premature stop codons and the expression of non-functional proteins or by insertion of nucleotides.

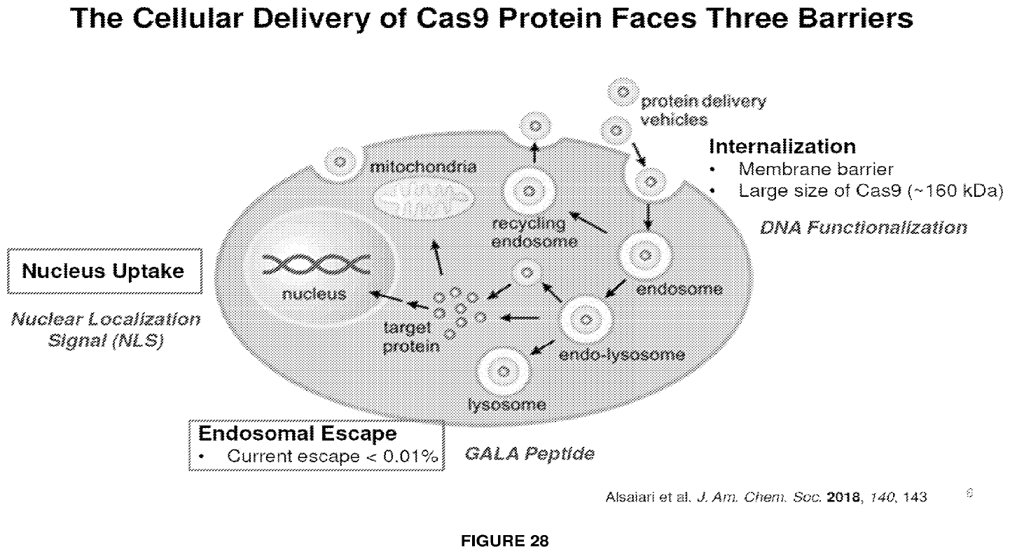

As depicted in FIG. 28, there are barriers to cellular entry of a gene editing protein. These barriers include internalization of the gene editing protein (due to the membrane barrier and the large size of gene editing proteins), how to achieve nuclear uptake of the gene editing protein, and how the gene editing protein can escape the endosome. Thus, in any of the aspects or embodiments of the disclosure, the gene editing protein is part of a “fused” protein. The term “fused” in this sense refers, in various aspects, to a protein comprising or consisting of the following elements fused together in order from N-terminus to C-terminus: (i) one or more GALA peptides; (ii) a gene editing protein, and (iii) a nuclear localization signal (NLS). In some aspects, the fused protein comprises or consists of the following elements fused together in order from N-terminus to C-terminus: (i) a gene editing protein, and (ii) a nuclear localization signal (NLS). The gene editing portion of the fused protein can be any gene editing protein known in the art and/or described herein, for example and without limitation a CRISPR-associated protein (Cas). In various embodiments, the Cas is Cas9, Cas12, Cas13, or a combination thereof. In some embodiments, the Cas9 is as described in Harrington, L. B., Paez-Espino, D., Staahl, B. T. et al. A thermostable Cas9 with increased lifetime in human plasma. Nat Commun 8, 1424 (2017). https://doi.org/10.1038/s41467-017-01408-4, incorporated by reference herein in its entirety. In some embodiments, the Cas9 comprises or consists of an amino acid sequence that is at least 95% identical to the amino acid sequence as set out in SEQ ID NO: 1 or SEQ ID NO: 25. In some embodiments, the Cas12 comprises or consists of an amino acid sequence that is at least 95% identical to the amino acid sequence as set out in SEQ ID NO: 27 (Strecker J, Jones S, Koopal B, Schmid-Burgk J, Zetsche B, Gao L, Makarova K S, Koonin E V, Zhang F. Nat Commun. 2019 Jan. 22; 10(1):212. doi: 10.1038/s41467-018-08224-4. 10.1038/s41467-018-08224-4 PubMed 30670702). In some embodiments, the Cas13 comprises or consists of an amino acid sequence that is at least 95% identical to the amino acid sequence as set out in SEQ ID NO: 29 (Smargon A A, Cox D B, Pyzocha N K, Zheng K, Slaymaker I M, Gootenberg J S, Abudayyeh O A, Essletzbichler P, Shmakov S, Makarova K S, Koonin E V, Zhang F. Mol Cell. 2017 Feb. 16; 65(4):618-630.e7. doi: 10.1016/j.molcel.2016.12.023. Epub 2017 Jan. 5. 10.1016/j.molcel.2016.12.023 PubMed 28065598). GALA peptides are known in the art (see, e.g., Schach et al., J. Am. Chem. Soc. 2015, 137, 38, 12199-12202, incorporated by reference herein in its entirety) and are described herein. The disclosure contemplates that in various embodiments, a fused protein comprises or consists of 1, 2, 3, 4, or 5 GALA peptides in tandem. In some embodiments, the N-terminus of a fused protein of the disclosure comprises or consists of 3 GALA peptides in tandem. In some embodiments, the N-terminus of a fused protein of the disclosure comprises or consists of 3 GALA peptides in tandem, wherein each GALA peptide comprises or consists of the amino acid sequence set forth in SEQ ID NO: 22. In any of the aspects or embodiments of the disclosure, the C-terminus of a fused protein as described herein comprises or consists of a NLS sequence. NLS sequences are known in the art (see, e.g., Cutrona, G., Carpaneto, E., Ulivi, M. et al. Effects in live cells of a c-myc anti-gene PNA linked to a nuclear localization signal. Nat Biotechnol 18, 300-303 (2000). https://doi.org/10.1038/73745, incorporated by reference herein in its entirety). In some embodiments, the NLS sequence is derived from the NLS of the SV40 virus large T-antigen and comprises or consists of the amino acid sequence PKKKRKV (SEQ ID NO: 23). In some embodiments, the NLS comprises or consists of the amino acid sequence KRTADGSEFESPKKKRKV (SEQ ID NO: 28). The disclosure also provides compositions comprising a fused protein as described herein and a pharmaceutically acceptable carrier. Fused proteins provided by the disclosure may be used in any of the ProSNAs, SNAs, compositions, and/or methods described herein. Thus, in some aspects, a ProSNA of the disclosure comprises (a) a protein core that comprises a fused protein; and (b) a shell of oligonucleotides attached to the protein core. In further aspects, the disclosure provides a SNA comprising (a) a nanoparticle core; (b) a shell of oligonucleotides attached to the external surface of the nanoparticle core; and (c) a fused protein.

Through in vitro studies using Streptococcus pyogenes type II CRISPR/Cas system it has been shown that the only components required for efficient CRISPR/Cas-mediated target DNA or genome modification are a Cas nuclease (e.g., a Cas9 nuclease), CRISPR RNA (crRNA) and trans-activating crRNA (tracrRNA). The wild-type mechanism of CRISPR/Cas-mediated DNA cleavage occurs via several steps. Transcription of the CRISPR array, containing small fragments (20-30 base-pairs) of the encountered (or target) DNA, into pre-crRNA, which undergoes maturation through the hybridization with tracrRNA via direct repeats of pre-crRNA. The hybridization of the pre-crRNA and tracrRNA, known as guide RNA (gRNA or sgRNA), associates with the Cas nuclease forming a ribonucleoprotein complex, which mediates conversion of pre-crRNA into mature crRNA. Mature crRNA:tracrRNA duplex directs Cas9 to the DNA target consisting of the protospacer and the requisite protospacer adjacent motif (CRISPR/cas protospacer-adjacent motif; PAM) via heteroduplex formation between the spacer region of the crRNA and the protospacer DNA on the host genome. The Cas9 nuclease mediates cleavage of the target DNA upstream of PAM to create a double-stranded break within the protospacer or a strand-specific nick using mutated Cas9 nuclease whereby one DNA strand-specific cleavage motif is mutated.

Thus, in various aspects involving gene editing, a SNA of the disclosure (e.g., ProSNA, LNP-SNA, LSNA) comprises a DNA or RNA gene editor substrate (e.g., a guide RNA) in addition to a gene editing protein, wherein the DNA or RNA gene editor substrate is, in various embodiments, attached to the surface of the SNA or encapsulated within the SNA. In some embodiments, a SNA that comprises a gene editing protein is delivered separately from the DNA or RNA gene editor substrate.

Other RNA-guided nucleases from related CRISPR systems that have also been adapted for programmable nucleic acid cleavage include Staphylococcus aureus Cas9 (SaCas9), CRISPR from Prevotella or Franciscella I (CpfI), Geobacillus Cas9 (GeoCas9), Campylobacter jejuni Cas9 (CjCas9), metagenomically derived CRISPR-CasX and CRISPR-CasY, CRISPR-Cas3, and CRISPR-C2c2, which cleaves RNA.

The CRISPR/Cas system has been modified to perform a number of functions besides gene knockout and editing, three examples of which are described below. Catalytically inactivated Cas9 (dCas9) has been fused to transcriptional activation and repression domains, thereby enabling programmable control of gene expression [Gilbert et al., Cell 154, 442-451 (2013); Zalatan et al., Cell 160, 339-350 (2015)]. The dCas9 transcriptional activator in particular enables novel screens analogous to siRNA or CRISPR knockout libraries, but where genes are over-expressed [Gilbert et al., Cell 159, 647-61 (2014)]. dCas9 fused to fluorescent proteins enable microscopic tracking of specific sites in the genome and study of sequence-specific nuclear organization [Chen et al., Cell 155, 1479-91 (2013)]. Finally, active Cas9 can be targeted to cleave a variety of nonfunctional genomic regions in a zygote, and the frequency and sequence of the mutation in each cell of the mature organism can be used to track lineages of cell differentiation during embryonic development [Mckenna et al., Science 42, 237-241 (2016)].

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA interacting enzyme (e.g., a methylase, a topoisomerase, an integrase, a transposase, or a ligase), a transcription activator or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

Accordingly, in some aspects, the disclosure provides SNAs (e.g., ProSNAs, LSNAs, LNP-SNAs) for use in the delivery of gene editing proteins. In various embodiments, the gene editing protein(s) are in a ribonucleoprotein (RNP) complex. The ribonucleoprotein (RNP) complex encapsulated in a SNA comprises, in various embodiments, CRISPR-associated protein 9 (Cas9) (SEQ ID NO: 1 or SEQ ID NO: 25), CRISPR RNA (crRNA), trans-activating crRNA (tracrRNA), and/or Transcription Activator-like Effector Nucleases (TALENs). In some embodiments, the Cas9 utilized in the compositions and methods of the disclosure is EnGen® Cas9 NLS, S. pyogenes (New England Biolabs Catalog Number M0646T). In any of the aspects or embodiments of the disclosure, a nucleotide or amino acid sequence of the disclosure comprises or consists of a sequence that is at least 80%, at least 85%, at least 90%, at least 95%, at least 99%, or 100% identical to a reference or wild type sequence. In any of the aspects or embodiments of the disclosure, the gene editing protein comprises or consists of an amino acid sequence that is at least 80%, at least 85%, at least 90%, at least 95%, at least 99%, or 100% identical to a reference or wild type sequence. In various embodiments, the gene editing protein is a Cas9 protein comprising or consisting of an amino acid sequence that is at least 80%, at least 85%, at least 90%, at least 95%, at least 99%, or 100% identical to to a reference or wild type Cas9 sequence. Thus, in various embodiments, the gene editing protein is a Cas9 protein comprising or consisting of an amino acid sequence that is at least 80%, at least 85%, at least 90%, at least 95%, at least 99%, or 100% identical to SEQ ID NO: 1 or SEQ ID NO: 25. In further embodiments, the gene editing protein is a Cas12 protein comprising or consisting of an amino acid sequence that is at least 80%, at least 85%, at least 90%, at least 95%, at least 99%, or 100% identical to SEQ ID NO: 27. In further embodiments, the gene editing protein is a Cas13 protein comprising or consisting of an amino acid sequence that is at least 80%, at least 85%, at least 90%, at least 95%, at least 99%, or 100% identical to SEQ ID NO: 29.

### SPHERICAL NUCLEIC ACIDS (SNAS)

As described herein, spherical nucleic acids (SNAs) are a unique class of nanomaterials comprising a spherical nanoparticle core functionalized with a highly oriented oligonucleotide shell. The oligonucleotide shell comprises one or more oligonucleotides attached to the external surface of the nanoparticle core. In various embodiments, the shell of oligonucleotides comprises an inhibitory oligonucleotide, an immunostimulatory oligonucleotide, a gene editor substrate DNA or RNA, a targeting oligonucleotide, or a combination thereof. The nanoparticle core can either be organic (e.g., a liposome), inorganic (e.g., gold, silver, or platinum), polymer-based (e.g., a poly (lactic-co-glycolic acid) (PLGA) particle), or hollow (e.g., silica-based). In various embodiments of the disclosure, the nanoparticle core is a protein (protein-core SNA (ProSNA)), a liposome (liposomal SNA (LSNA)), or a lipid nanoparticle (LNP-SNA).

The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis (see, e.g., U.S. Patent Application Publication No. 2010/0233270, incorporated by reference herein in its entirety).

### Protein-Core Spherical Nucleic Acids (ProSNAs)

Recently, protein spherical nucleic acids (ProSNAs), which comprise a dense shell of oligonucleotides attached (e.g., covalently attached) to a protein core, have emerged as exciting new architectures with diverse biological applications in protein delivery, assembly, and intracellular detection [Brodin, J. D.; Sprangers, A. J.; McMillan, J. R.; Mirkin, C. A. DNA-Mediated Cellular Delivery of Functional Enzymes. J. Am. Chem. Soc. 2015, 137 (47), 14838 -14841; Kusmierz, C. D.; Bujold, K. E.; Callmann, C. E.; Mirkin, C. A. Defining the Design Parameters for in Vivo Enzyme Delivery Through Protein Spherical Nucleic Acids. ACS Cent. Sci. 2020, 6 (5), 815-822]. The dense shell of oligonucleotides promotes cellular uptake, physiological stability and biocompatibility of protein relative to their individual components [Giljohann, D. A.; Seferos, D. S.; Patel, P. C.; Millstone, J. E.; Rosi, N. L.; Mirkin, C. A. Oligonucleotide Loading Determines Cellular Uptake of DNA-Modified Gold Nanoparticles. Nano Lett. 2007, 7 (12), 3818-3821]. This enhanced cellular internalization of SNAs is derived from the three-dimension architecture of the conjugates and its ability to engage scavenger receptors on the surfaces of most cells. Importantly, the favorable biological properties of SNAs are independent of their protein cores, which can therefore be chosen for protein delivery genome editing applications.

A “protein-core” as used herein comprises a gene editing protein. Thus, in any of the aspects or embodiments of the disclosure, a gene editing protein of the disclosure generally functions as the “core” of the protein-core SNA (SNA). A protein is a molecule comprising one or more polymers of amino acids. In various embodiments of the disclosure, a protein-core comprises or consists of a single protein (i.e., a single polymer of amino acids), a multimeric protein, a peptide (e.g., a polymer of amino acids that between about 2 and 50 amino acids in length), or a synthetic fusion protein of two or more proteins. Synthetic fusion proteins include, without limitation, an expressed fusion protein (expressed from a single gene) and post-expression fusions where proteins are conjugated together chemically. In any of the aspects or embodiments of the disclosure, a protein-core comprises or consists of a gene editing protein. Proteins are understood in the art and may be either naturally occurring or non-naturally occurring.

Protein-Core SNA Synthesis. The disclosure provides compositions and methods in which one or more oligonucleotides is associated with and/or attached to the surface of a protein-core SNA via a linker. The linker can be, in various embodiments, a cleavable linker, a non-cleavable linker, a traceless linker, or a combination thereof. In some embodiments, a cleavable linker is sensitive to (and is cleaved in response to) a reducing agent (e.g., glutathione (GSH), dithiothreitol (DTT)) or a reducing environment (e.g., inside a cell). In various embodiments, a cleavable linker is sensitive to (and is cleaved in response to) various chemical stimuli such as, for example, acidity (e.g., low pH), an enzyme (e.g., peptidase), light (e.g., NIR laser), and/or hydrolysis.

The linker links the protein-core to the oligonucleotide in the disclosed protein-core SNA (i.e., protein-core-LINKER-Oligonucleotide). In various embodiments, a single oligonucleotide is attached to a linker. In further embodiments, more than one oligonucleotide (e.g., two, three, or more) is attached to a single linker. In general, linkers contemplated by the disclosure include the following, which may be used solely or in combination in the ProSNAs of the disclosure: amide, thioether, triazole, oxime, urea, and thiourea. Some specifically contemplated linkers include carbamate alkylene, carbamate alkylenearyl dithiolate linkers, amide alkylene dithiolate linkers, amide alkylenearyl dithiolate linkers, and amide alkylene succinimidyl linkers. In some cases, the linker comprises-NH—C(O)—O—C₂₋₅alkylene-S-—S—C₂₋₇alkylene- or —NH—C(O)—C₂₋₅alkylene-S—S—C₂₋₇alkylene-. The carbon alpha to the —S—S— moiety can be branched, e.g., —C(XA)(XB)—S—S— or —S—S—C(YA)(YB)— or a combination thereof, where XA, XB, YA and YB are independently H, Me, Et, or iPr. The carbon alpha to the protein can be branched, e.g., —C(XA)(XB)—C₂₋₄alkylene-S—S—, where XA and XB are H, Me, Et, or iPr. In some cases, the linker is —NH—C(O)—O—CH₂₋₄—Ar—S—S—C₂₋₇alkylene-, and Ar is a meta- or para-substituted phenyl. In some cases, the linker is —NH—C(O)—C₂₋₄alkylene-N-succinimidyl-S—C₂₋₆alkylene-.

Additional linkers contemplated by the disclosure include those described in International Patent Publication No. WO 2018/213585, incorporated herein by reference in its entirety. In some embodiments, the linker is an SH linker, SM linker, SE linker, or SI linker. The disclosure contemplates multiple points of attachment for oligonucleotides on a protein-core.

An oligonucleotide of the disclosure may be modified at either the 5′ terminus or the 3′ terminus for attachment to a protein core.

An oligonucleotide of the disclosure can be modified at a terminus with an alkyne moiety, e.g., a DBCO-type moiety for reaction with the azide of the protein surface:

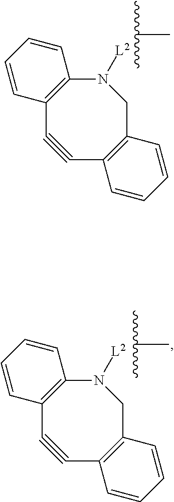

where L is a linker to a terminus of the polynucleotide. L² can be C₁₋₁₀alkylene, —C(O)—C₁₋₁₀alkylene-Y—, and —C(O)—C₁₋₁₀alkylene-Y—C₁₋₁₀alkylene- (OCH₂CH₂)ₘ—Y—; wherein each Y is independently selected from the group consisting of a bond, C(O), O, NH, C(O)NH, and NHC(O); and m is 0, 1, 2, 3, 4, or 5. For example, the DBCO functional group can be attached via a linker having a structure of

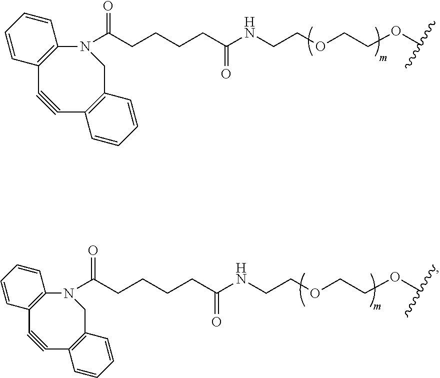

where the terminal “O” is from a terminal nucleotide on the polynucleotide. Use of this DBCO-type moiety results in a structure between the polynucleotide and the protein, in cases where a

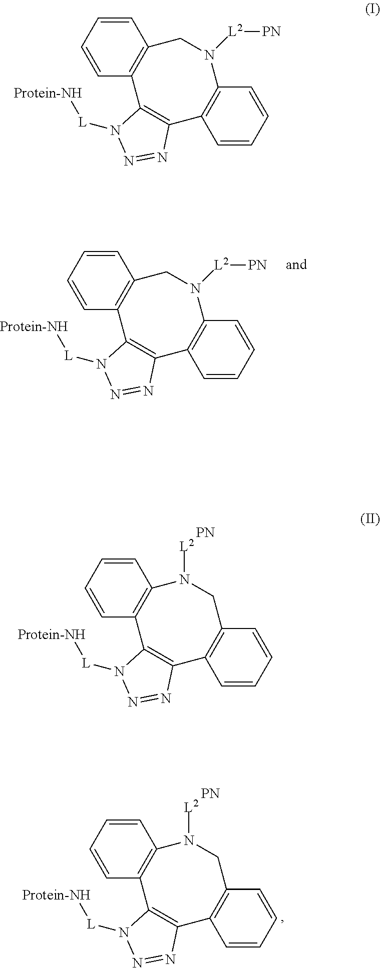

surface amine is modified, of:
where L and L² are each independently selected from C₁₋₁₀alkylene, —C(O)—C₁₋₁₀alkylene-Y—, and —C(O)—C₁₋₁₀alkylene-Y—C₁₋₁₀alkylene-(OCH₂CH₂)ₘ—Y—; each Y is independently selected from the group consisting of a bond, C(O), O, NH, C(O)NH, and NHC(O); m is 0, 1, 2, 3, 4, or 5; and PN is the polynucleotide. Similar structures where a surface thiol or surface carboxylate of the protein are modified can be made in a similar fashion to result in comparable linkage structures.

The protein can be modified at a surface functional group (e.g., a surface amine, a surface carboxylate, a surface thiol) with a linker that terminates with an azide functional group: Protein-X-L-N₃, X is from a surface amino group (e.g., —NH—), carboxylic group (e.g., —C(O)— or —C(O)O—), or thiol group (e.g., —S—) on the protein; L is selected from C₁₋₁₀alkylene, —Y—C(O)—C₁₋₁₀alkylene-Y—, and —Y—C(O)—C₁₋₁₀alkylene-Y—C₁₋₁₀alkylene-(OCH₂CH₂)ₘ—Y—; each Y is independently selected from the group consisting of a bond, C(O), O, NH, C(O)NH, and NHC(O); and m is 0, 1, 2, 3, 4, or 5. Introduction of the “L-N₃” functional group to the surface moiety of the protein can be accomplished using well-known techniques. For example, a surface amine of the protein can be reacted with an activated ester of a linker having a terminal N₃ to form an amide bond between the amine of the protein and the carboxylate of the activated ester of the linker reagent.

The oligonucleotide can be modified to include an alkyne functional group at a terminus of the oligonucleotide: Oligonucleotide-L₂—X—≡—R; L² is selected from C₁₋₁₀alkylene, —C(O)—C₁₋₁₀alkylene-Y—, and —C(O)—C₁₋₁₀alkylene-Y—C₁₋₁₀alkylene-(OCH₂CH₂)ₘ—Y—; each Y is independently selected from the group consisting of a bond, C(O), O, NH, C(O)NH, and NHC(O); m is 0, 1, 2, 3, 4, or 5; and X is a bond and R is H or C₁₋₁₀alkyl; or X and R together with the carbons to which they are attached form a 8-10 membered carbocyclic or 8-10 membered heterocyclic group. In some cases, the polynucleotide has a structure

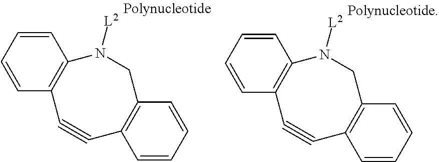

The protein, with the surface modified azide, and the polynucleotide, with a terminus modified to include an alkyne, can be reacted together to form a triazole ring in the presence of a copper (II) salt and a reducing agent to generate a copper (I) salt in situ. In some cases, a copper (I) salt is directly added. Contemplated reducing agents include ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, a sulfite compound, a stannous compound, a ferrous compound, sodium amalgam, tris(2-carboxyethyl)phosphine, hydroquinone, and mixtures thereof.

The surface functional group of the protein can be attached to the oligonucleotide using other attachment chemistries. For example, a surface amine can be directly conjugated to a carboxylate or activated ester at a terminus of the oligonucleotide, to form an amide bond. A surface carboxylate can be conjugated to an amine on a terminus of the oligonucleotide to form an amide bond. Alternatively, the surface carboxylate can be reacted with a diamine to form an amide bond at the surface carboxylate and an amine at the other terminus. This terminal amine can then be modified in a manner similar to that for a surface amine of the protein. A surface thiol can be conjugated with a thiol moiety on the polynucleotide to form a disulfide bond. Alternatively, the thiol can be conjugated with an activated ester on a terminus of a polynucleotide to form a thiocarboxylate. Alternatively, the thiol can be conjugated with a Michael acceptor (e.g., a succinimide) on a terminus of a polynucleotide to form a thioether.

A general, a representative procedure for synthesizing protein-core SNAs (ProSNAs) includes attaching a desired amount of oligonucleotide to the surface of the protein. Attachment is performed by iterating over a two-step process: (1) attachment of linker to the surface of the protein and purification; (2) attachment of oligonucleotide (e.g., . . . DNA) to the protein-conjugated linkers and purification. These two steps are repeated until a desired amount of oligonucleotide is attached to the protein. It will be understood that the foregoing procedure is exemplary in nature.

### Lipid Nanoparticle Spherical Nucleic Acids (LNP-SNAs)

Lipid nanoparticle spherical nucleic acids (LNP-SNAs) are comprised of a lipid nanoparticle core decorated with oligonucleotides. The lipid nanoparticle core comprises a gene editing protein, an ionizable lipid, a phospholipid, a sterol, and a lipid-polyethylene glycol (lipid-PEG) conjugate. The oligonucleotide shell comprises one or a plurality of oligonucleotides attached to the external surface of the lipid nanoparticle core. The spherical architecture of the oligonucleotide shell confers unique advantages over traditional nucleic acid delivery methods, including entry into nearly all cells independent of transfection agents, resistance to nuclease degradation, sequence-based function, targeting, and diagnostics.

Accordingly, in various aspects, the disclosure provides a lipid nanoparticle spherical nucleic acid (LNP-SNA) comprising (a) a lipid nanoparticle core; (b) a shell of oligonucleotides attached to the external surface of the lipid nanoparticle core; and (c) a gene editing protein. Thus, the LNP-SNA comprises a gene editing protein, an ionizable lipid, a phospholipid, a sterol, and a lipid-polyethylene glycol (lipid-PEG) conjugate. In some embodiments, the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), similar lipid/lipidoid structures, or a combination thereof. In some embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), or a combination thereof. In further embodiments, the sterol is 3β-Hydroxycholest-5-ene (Cholesterol), 9,10-Secocholesta-5,7,10(19)-trien-3β-ol (Vitamin D3), 9,10-Secoergosta-5,7,10(19),22-tetraen-3β-ol (Vitamin D2), Calcipotriol, 24-Ethyl-5,22-cholestadien-3β-ol (Stigmasterol), 22,23-Dihydrostigmasterol (β-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids. In some embodiments, the lipid-polyethylene glycol (lipid-PEG) conjugate comprises 2000 Dalton (Da) polyethylene glycol. In further embodiments, the lipid-polyethylene glycol (lipid-PEG) conjugate is lipid-PEG-maleimide. In still further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof.

Oligonucleotides contemplated for use according to the disclosure include those attached to a nanoparticle core through any means (e.g., covalent or non-covalent attachment). In any of the aspects or embodiments of the disclosure an oligonucleotide is attached to the exterior of a lipid nanoparticle core via a covalent attachment of the oligonucleotide to a lipid-polyethylene glycol (lipid-PEG) conjugate. In some embodiments, 10%, 20%, 30%, 40%, 50%, 60%, 70%, 80%, 90% or 100% of the oligonucleotides in the shell of oligonucleotides are covalently attached to the exterior of the lipid nanoparticle core through the lipid-PEG conjugate. In various embodiments, one or more oligonucleotides in the oligonucleotide shell is attached to the exterior of the lipid nanoparticle core through a lipid anchor group. The lipid anchor group is, in various embodiments, attached to the 5′- or 3′-end of the oligonucleotide. In various embodiments, the lipid anchor group is cholesterol or tocopherol.

In any of the aspects or embodiments of the disclosure, a LNP-SNA is synthesized such that a gene editing protein is encapsulated in the lipid nanoparticle core and a shell of oligonucleotides is attached to the exterior of the lipid nanoparticle core. In general and by way of example, lipid nanoparticles (LNPs) may be formulated by diluting the lipids and sterols in ethanol.

### Liposomal Spherical Nucleic Acids (LSNAs)

Liposomes are spherical, self-closed structures in a varying size range comprising one or several hydrophobic lipid bilayers with a hydrophilic core. The diameter of these lipid based carriers range from 0.15-1 micrometers, which is significantly higher than an effective therapeutic range of 20-100 nanometers. Liposomes termed small unilamellar vesicles (SUVs), can be synthesized in the 20-50 nanometer size range, but encounter challenges such as instability and aggregation leading to inter-particle fusion. This inter-particle fusion limits the use of SUVs in therapeutics.

Liposomal spherical nucleic acids (LSNAs) are an attractive platform for therapeutic delivery due to their chemically tunable structures, biocompatibility, and ability to rapidly enter cells without transfection reagents. The instant disclosure provides methods for delivering gene editing proteins into cells by encapsulating them in LSNAs. Encapsulated gene editing enzymes remain enzymatically active, and rapidly enter mammalian cells. These properties make this new form of LSNAs a delivery vehicle for gene editing therapeutics.

Previous SNA-mediated protein delivery strategies require chemical modification of amino acids on the protein, which can inhibit protein function. Proteins encapsulated in LSNAs can be delivered into cells without any chemical modifications. Further, cationic lipid-mediated strategies for protein delivery require an anionic protein complex. SNA-mediated delivery, however, uses neutral phospholipids, and should not require anionic proteins. Thus, this method also lends itself to the delivery of positively charged proteins, such as TALENs.

Accordingly, in some aspects the disclosure contemplates use of the LSNAs disclosed herein, comprising gene editing enzymes (e.g., CRISPR-associated protein 9 (Cas9) (Jinek et al., (2012) Science. 816-821; Zuris et al., Nat Biotechnol. 2015 January; 33(1):73-80, incorporated herein by reference in their entireties), CRISPR RNA (crRNA), and trans-activating crRNA (tracrRNA), Transcription Activator-like Effector Nucleases (TALENs)) and surface-functionalized oligonucleotides in methods of gene editing.

Accordingly, the present disclosure provides LSNAs for use in methods including but not limited to the in vitro or in vivo delivery of gene editing proteins (e.g., to cells). Liposomal particles, for example as disclosed in International Patent Application No. PCT/US2014/068429 (incorporated by reference herein in its entirety, particularly with respect to the discussion of liposomal particles) are also contemplated by the disclosure. Liposomal particles of the disclosure have at least a substantially spherical geometry, an internal side and an external side, and comprise a lipid bilayer. Thus, in various aspects, the disclosure provides a spherical nucleic acid (SNA) comprising (a) a liposomal core; (b) a shell of oligonucleotides attached to the external surface of the liposomal core; and (c) a gene editing protein. The lipid bilayer comprises a plurality of lipid groups comprising, in various embodiments, a lipid from the phosphocholine family of lipids or the phosphoethanolamine family of lipids. Lipids contemplated by the disclosure include, without limitation, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z -octadecenoyl)-sn-glycero-3-phosphoethanolamine (DOPE), 1,2-dihexadecanoyl-sn-glycero-3-phosphoethanolamine (DPPE), 1,2-dioleoyl-sn-glycero-3-phospho-(1′-rac-glycerol) (DOPG), 1,2-distearoyl-sn-glycero-3-phosphoethanolamine (DSPE), cardiolipin, lipid A, a combination thereof. In various embodiments, at least one oligonucleotide in the shell of oligonucleotides is attached to the exterior of the liposomal core through a lipid anchor group. In further embodiments, the lipid anchor group is attached to the 5′ end or the 3′ end of the at least one oligonucleotide. In still further embodiments, the lipid anchor group is tocopherol or cholesterol. Thus, in various embodiments, at least one (or all) of the oligonucleotides in the shell of oligonucleotides is an oligonucleotide-lipid conjugate containing a lipid anchor group, wherein said lipid anchor group is adsorbed into the lipid bilayer. The lipid anchor group comprises, in various embodiments, tocopherol, palmitoyl, dipalmitoyl, stearyl, distearyl, or cholesterol. In further aspects, the disclosure provides a LSNA having a substantially spherical geometry and comprising a lipid bilayer comprising a plurality of lipid groups; a ribonucleoprotein (RNP) complex encapsulated in the liposomal particle, the RNP comprising a gene editing protein (e.g., CRISPR-associated protein 9 (Cas9)) and guide RNA; and one or more oligonucleotides on the surface of the LSNA.

With respect to the surface density of oligonucleotides on the surface of a LSNA of the disclosure, it is contemplated that a LSNA as described herein comprises from about 1 to about 400 oligonucleotides on its surface. In various embodiments, a LSNA comprises from about 10 to about 100, or from 10 to about 90, or from about 10 to about 80, or from about 10 to about 70, or from about 10 to about 60, or from about 10 to about 50, or from about 10 to about 40, or from about 10 to about 30, or from about 10 to about 20, or from about 50 to about 100, or from about 60 to about 100, or from about 70 to about 100, or from about 80 to about 100, or from about 90 to about 100 oligonucleotides on its surface. In further embodiments, a LSNA comprises or consists of at least about 5, 10, 20, 30, 40, 50, 60, 70, 80, 90, 100, 150, 200, 250, 300, 350, or 400 oligonucleotides on its surface. In some embodiments, a LSNA comprises or consists of 70 oligonucleotides on its surface. Additional surface densities for SNAs are described herein below.

In some aspects, an architecture comprising a tocopherol modified oligonucleotide is disclosed. In various embodiments, tocopherol is contemplated to be on the 5′ end or the 3′ end of an oligonucleotide or modified form thereof. A tocopherol-modified oligonucleotide comprises a lipophilic end and a non-lipophilic end. The lipophilic end comprises tocopherol, and may be chosen from the group consisting of a tocopherol derivative, alpha-tocopherol, beta-tocopherol, gamma-tocopherol and delta-tocopherol. The lipophilic end, in further embodiments, comprises palmitoyl, dipalmitoyl, stearyl, cholesterol, or distearyl.

In further aspects, the disclosure contemplates that cholesterol or phospholipids are used instead of tocopherol. Cholesterol is attached in solid phase oligonucleotide synthesis, where it is mixed with the prepared liposomes to form SNAs. In some embodiments, liposomes composed of 95% 1,2-dioleoyl-sn-glycero-3 phosphatidylcholine (DOPC) and 5% 1,2-dipalmitoyl-sn-glycero-3-phosphoethanolamine-N-(6-azidohexanoyl) (DPPE-Azide) are prepared as described below. Then DBCO-modified oligonucleotides are added, which react with the azide lipid to functionalize the surface.

In still further aspects, a phospholipid conjugated oligonucleotide is prepared as follows: First, a phosphatidylethanolamine lipid, such as DOPE, is reacted with succinimidyl 4-(p-maleimidophenyl)butyrate (SMPB) by mixing 25 mg/mL lipid, 1 equivalent SMPB and 1 equivalent of N,N-Diisopropylethylamine in chloroform. The mixture is reacted overnight. Next, the product is purified by flash chromatography using silica column (solvent A: dichloromethane, solvent B: methanol). The thiol-modified oligonucleotide (3′ or 5′ end modified) is reduced with 0.2M DTT and 0.1 M phosphate buffer (pH 8) at 40° C. for 2 hours. The oligonucleotide is then purified in a size exclusion column using water. The phosphatidylethanolamine-SMPB lipid is dried over nitrogen gas and dissolved in ethanol in the same volume as the oligonucleotide. The oligonucleotide is then mixed with the lipid such that the reaction is 50:50 water and ethanol. This mixture is reacted overnight, and the excess lipid is extracted by washing the reaction mixture with chloroform three times. Next, the aqueous phase and the interface are dried and dissolved in water. All lipid-conjugated oligonucleotides as disclosed herein are contemplated to be used interchangeably in the preparation of LSNAs. The non-lipophilic end of the tocopherol-modified oligonucleotide is an oligonucleotide as described herein.

Methods of making oligonucleotides comprising a lipid anchor are disclosed herein. For example, first an oligonucleotide and phosphoramidite-modified-tocopherol are provided. Then, the oligonucleotide is exposed to the phosphoramidite-modified-tocopherol to create the tocopherol modified oligonucleotide. While not meant to be limiting, any chemistry known to one of skill in the art can be used to attach the tocopherol (or any lipid anchor) to the oligonucleotide, including amide linking or click chemistry.

The disclosure also provides methods of making LSNAs. In some embodiments, a phospholipid, solvent, and a tocopherol modified oligonucleotide are provided. Then, the phospholipid is added to the solvent to form a first mixture comprising liposomes. The size of the liposomes in the first mixture is between about 100 nanometers and about 150 nanometers. Next, the liposomes are disrupted to create a second mixture comprising liposomes and small unilamellar vesicles (SUV). The size of the liposomes and SUVs in the second mixture is between about 20 nanometers and about 150 nanometers. Next, the SUVs having a particle size between about 20 nanometers and about 50 nanometers are isolated from the second mixture. Finally, the tocopherol modified oligonucleotide is added to the isolated SUVs to make a liposomal particle. In various embodiments, the diameter of the LSNAs created by a method of the disclosure is less than or equal to about 50 nanometers. In some embodiments, a plurality of LSNAs is produced and the particles in the plurality have a mean diameter of less than or equal to about 50 nanometers (e.g., about 5 nanometers to about 50 nanometers, or about 5 nanometers to about 40 nanometers, or about 5 nanometers to about 30 nanometers, or about 5 nanometers to about 20 nanometers, or about 10 nanometers to about 50 nanometers, or about 10 nanometers to about 40 nanometers, or about 10 nanometers to about 30 nanometers, or about 10 nanometers to about 20 nanometers). In further embodiments, the particles in the plurality of LSNAs created by a method of the disclosure have a mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers.

In some aspects, the method comprises: (1) adding 1× PBS to dry lipids to a final concentration of 1-25 mg/mL (thus, in various embodiments, the final concentration is about 1, 2, 3, 4, 5, 6, 7, 8, 9, 10, 11, 12, 13, 14, 15, 16, 17, 18, 19, 20, 21, 22, 23, 24, or 25 mg/ml); (2) freezing rapidly in liquid nitrogen and thawing in a bath sonicator 3 times; (3) extruding through 200, 100, 80, 50 and 30 nm filters. Double filters are used and typically passed 2-10 times through each filter. In some embodiments, the process is stopped at 50 nm, but if 30 nm structures are desired, then the 30 nm filter is additionally added. In further aspects, when 30 nm liposomes are desired, one probe sonicates after step (2). Next, the liposomes are centrifuged at 21000× g for 10 minutes to remove metal shavings that come off in sonication and the mixture is extruded through a 30 nm filter as described in step (3).

Thus, in some aspects the disclosure provides a method of making a LSNA, comprising adding a phospholipid to a solvent to form a first mixture, said first mixture comprising a plurality of liposomes; disrupting said plurality of liposomes to create a second mixture, said second mixture comprising a liposome and a small unilamellar vesicle (SUV); isolating said SUV from said second mixture, said SUV having a particle size between about 20 nanometers and 50 nanometers; and adding an oligonucleotide or a plurality of oligonucleotides to the isolated SUV to make the LSNA.

### Oligonucleotides

The disclosure provides spherical nucleic acids (e.g., ProSNAs, LSNAS, LNP-SNAs) comprising a nanoparticle core and a shell of oligonucleotides attached to the exterior of the nanoparticle core. The shell of oligonucleotides comprises, in various embodiments, an inhibitory oligonucleotide, an immunostimulatory oligonucleotide, a targeting oligonucleotide, or a combination thereof. As described herein, in some embodiments the nanoparticle core comprises an encapsulated gene editing protein. Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded. In any aspects or embodiments of the disclosure, an oligonucleotide comprises a detectable marker.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e, a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where RH is selected from hydrogen and C_{1,4}-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂, —O—CH₂—CH=(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N=(including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH=(including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—O—, —O—P(O)₂—NR^{H}—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et, al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH2), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation. 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H -pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T, and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 5 nucleotides to about 100 nucleotides in length. More specifically, an oligonucleotide of the disclosure is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. In further embodiments, an oligonucleotide of the disclosure is about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In various embodiments, the shell of oligonucleotides attached to the exterior of the nanoparticle core of the SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. In various embodiments, the nanoparticle core comprises one or more oligonucleotides encapsulated therein.

In some embodiments, an oligonucleotide in the shell of oligonucleotides is an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Methods of attaching detectable markers (e.g., fluorophores, radiolabels) and therapeutic agents (e.g., an antibody) as described herein to an oligonucleotide are known in the art.

Spacers. In some aspects and embodiments, one or more oligonucleotides in the shell of oligonucleotides that is attached to the nanoparticle core of a SNA comprise a spacer. “Spacer” as used herein means a moiety that serves to increase distance between the nanoparticle core and the oligonucleotide, or to increase distance between individual oligonucleotides when attached to the nanoparticle core in multiple copies, or to improve the synthesis of the SNA. Thus, spacers are contemplated being located between an oligonucleotide and the nanoparticle core.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the nanoparticle core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

SNA surface density. Generally, a surface density of oligonucleotides that is at least about 2 pmoles/cm² will be adequate to provide a stable SNA. In some aspects, the surface density of a SNA of the disclosure (e.g., ProSNA, LSNA, LNP-SNA) is at least 15 pmoles/cm². Methods are also provided wherein the oligonucleotide is attached to the nanoparticle core of the SNA at a surface density of about 2 pmol/cm² to about 200 pmol/cm², or about 10 pmol/cm² to about 100 pmol/cm². In further embodiments, the surface density is at least about 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. In further embodiments, the surface density is less than about 2 pmol/cm², less than about 3 pmol/cm², less than about 4 pmol/cm², less than about 5 pmol/cm², less than about 6 pmol/cm², less than about 7 pmol/cm², less than about 8 pmol/cm², less than about 9 pmol/cm², less than about 10 pmol/cm², less than about 15 pmol/cm², less than about 19 pmol/cm², less than about 20 pmol/cm², less than about 25 pmol/cm², less than about 30 pmol/cm², less than about 35 pmol/cm², less than about 40 pmol/cm², less than about 45 pmol/cm², less than about 50 pmol/cm², less than about 55 pmol/cm², less than about 60 pmol/cm², less than about 65 pmol/cm², less than about 70 pmol/cm², less than about 75 pmol/cm², less than about t 80 pmol/cm², less than about 85 pmol/cm², less than about 90 pmol/cm², less than about 95 pmol/cm², less than about 100 pmol/cm², less than about 125 pmol/cm², less than about 150 pmol/cm², less than about 175 pmol/cm², less than about 200 pmol/cm², less than about 250 pmol/cm², less than about 300 pmol/cm², less than about 350 pmol/cm², less than about 400 pmol/cm², less than about 450 pmol/cm², less than about 500 pmol/cm², less than about 550 pmol/cm², less than about 600 pmol/cm², less than about 650 pmol/cm², less than about 700 pmol/cm², less than about 750 pmol/cm², less than about 800 pmol/cm², less than about 850 pmol/cm², less than about 900 pmol/cm², less than about 950 pmol/cm², or less than about 1000 pmol/cm².

Alternatively, the density of oligonucleotide attached to the SNA is measured by the number of oligonucleotides attached to the SNA. With respect to the surface density of oligonucleotides attached to a SNA of the disclosure, it is contemplated that a SNA as described herein comprises about 1 to about 2,500, or about 1 to about 500 oligonucleotides on its surface. In various embodiments, a SNA comprises about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20 oligonucleotides in the shell of oligonucleotides attached to the nanoparticle core. In some embodiments, a SNA comprises about 80 to about 140 oligonucleotides in the shell of oligonucleotides attached to the nanoparticle core. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In further embodiments, a SNA consists of 5, 10, 20, 30. 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In still further embodiments, the shell of oligonucleotides attached to the nanoparticle core of the SNA comprises 2, 3, 4, 5, 6, 7, 8, 9, 10, 11, 12, 13, 14, 15, 16, 17, 18, 19, 20 or more oligonucleotides. In some embodiments, the shell of oligonucleotides attached to the nanoparticle core of the SNA consists of 2, 3, 4, 5, 6, 7, 8, 9, 10, 11, 12, 13, 14, 15, 16, 17, 18, 19, or 20 oligonucleotides.

### Compositions

The disclosure also provides compositions that comprise a SNA of the disclosure, or a plurality thereof. In any of the aspects or embodiments of the disclosure, the composition further comprises a guide RNA. In some embodiments, the composition further comprises a pharmaceutically acceptable carrier. The term “carrier” refers to a vehicle within which the SNA as described herein is administered to a subject. Any conventional media or agent that is compatible with the SNAs according to the disclosure can be used. The term carrier encompasses diluents, excipients, adjuvants and a combination thereof. Pharmaceutically acceptable carriers are well known in the art (see, e.g., Remington's Pharmaceutical Sciences by Martin, 1975, the entire disclosure of which is herein incorporated by reference).

Exemplary “diluents” include water for injection, saline solution, buffers such as Tris, acetates, citrates or phosphates, fixed oils, polyethylene glycols, glycerine, propylene glycol or other synthetic solvents. Exemplary “excipients” include but are not limited to stabilizers such as amino acids and amino acid derivatives, polyethylene glycols and polyethylene glycol derivatives, polyols, acids, amines, polysaccharides or polysaccharide derivatives, salts, and surfactants; and pH-adjusting agents. In some embodiments, the SNAs provided herein comprise immunostimulatory oligonucleotides (for example and without limitation, a CpG oligonucleotide) as adjuvants. Other adjuvants known in the art may also be used in the compositions of the disclosure. For example, the adjuvant may be aluminum or a salt thereof, mineral oils, Freund adjuvant, vegetable oils, water-in-oil emulsion, mineral salts, small molecules (e.g., imiquimod, resiquimod), bacterial components (e.g., flagellin, monophosphoryl lipid A), or a combination thereof.

### Uses of SNAs in Gene Regulation

In some aspects of the disclosure, an oligonucleotide associated with a SNA (e.g., ProSNA, LNP-SNA, LSNA) inhibits the expression of a gene. Methods for inhibiting gene product expression provided herein include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In some aspects of the disclosure, it is contemplated that a SNA performs both a gene inhibitory function as well as an agent delivery function. In such aspects, an agent (e.g., a therapeutic agent) is associated with a SNA and the particle is additionally functionalized with one or more oligonucleotides designed to effect inhibition of target gene expression.

In various aspects, the methods include use of an oligonucleotide which is 100% complementary to the target polynucleotide, i.e., a perfect match, while in other aspects, the oligonucleotide is at least (meaning greater than or equal to) about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

It is understood in the art that the sequence of an antisense compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). The percent complementarity is determined over the length of the oligonucleotide. For example, given an antisense compound in which 18 of 20 nucleotides of the antisense compound are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The oligonucleotide utilized in such methods is either RNA or DNA. The RNA can be an inhibitory oligonucleotide, such as an inhibitory RNA (RNAi) that performs a regulatory function, and in various embodiments is selected from the group consisting of a small inhibitory RNA (siRNA), a single-stranded RNA (ssRNA), and a ribozyme. Alternatively, the RNA is microRNA that performs a regulatory function. The DNA is, in some embodiments, an antisense-DNA. In some embodiments, the RNA is a piwi-interacting RNA (piRNA).

### Uses of SNAs in Immune Regulation

Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines. chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 4, TLR 8 and TLR 9 that respond to specific oligonucleotides are located inside special intracellular compartments, called endosomes. The mechanism of modulation of, for example and without limitation, TLR 4, TLR 8 and TLR 9 receptors, is based on DNA-protein interactions.

As described herein, synthetic immunostimulatory oligonucleotides that contain CpG motifs that are similar to those found in bacterial DNA stimulate a similar response of the TLR receptors. Thus, CpG oligonucleotides of the disclosure have the ability to function as TLR agonists. Other TLR agonists contemplated by the disclosure include, without limitation, single-stranded RNA and small molecules (e.g., R848 (Resiquimod)). Therefore, immunomodulatory (e.g., immunostimulatory) oligonucleotides have various potential therapeutic uses, including treatment of immune deficiency and cancer. Thus, in some embodiments, a SNA of the disclosure is used in a method to modulate the activity of a toll-like receptor (TLR).

In some embodiments, a SNA of the disclosure (e.g., a ProSNA, LSNA, LNP-SNA) comprises an oligonucleotide that is a TLR antagonist. In some embodiments, the TLR antagonist is a single-stranded DNA (ssDNA).

In some embodiments, down regulation of the immune system involves knocking down the gene responsible for the expression of the Toll-like receptor. This antisense approach involves use of a SNA of the disclosure to inhibit the expression of any toll-like protein.

Accordingly, in some embodiments, methods of utilizing SNAs as described herein for modulating toll-like receptors are disclosed. The method either up-regulates or down-regulates the Toll-like-receptor activity through the use of a TLR agonist or a TLR antagonist, respectively. The method comprises contacting a cell having a toll-like receptor with a SNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Uses of SNAs to Treat a Disorder

In some embodiments, a SNA of the disclosure (e.g., ProSNA, LSNA, LNP-SNA) is used to treat a disorder. Thus, in some aspects, the disclosure provides methods of treating a disorder comprising administering an effective amount of a SNA of the disclosure to a subject (e.g., a human subject) in need thereof, wherein the administering treats the disorder. In various embodiments, the disorder is cancer, an infectious disease, a pulmonary disease, a gastrointestinal disease, a hematologic disease, a viral disease, an inflammatory disease, an autoimmune disease, a neurodegenerative disease, an inherited disease, cardiovascular disease, or a combination thereof. An “effective amount” of the SNA is an amount sufficient to, for example, effect gene editing and treat the disorder. An effective amount of the SNA is also the amount to, for example, inhibit gene expression, activate an innate immune response, or a combination thereof and treat the disorder. Thus, methods of activating an innate immune response are also contemplated herein, such methods comprising administering a SNA of the disclosure to a subject in need thereof in an amount effective to activate an innate immune response in the subject.

A SNA of the disclosure can be administered via any suitable route, such as parenteral administration, intramuscular injection, subcutaneous injection, intradermal administration, and/or mucosal administration such as oral or intranasal. Additional routes of administration include but are not limited to intravenous, intraperitoneal, intranasal administration, intra-vaginal, intra-rectal, and oral administration. A combination of different routes of administration, separately or at the same time, is also contemplated by the disclosure.

### Therapeutic Agents

The SNAs provided herein optionally further comprise a therapeutic agent, or a plurality thereof. The therapeutic agent is, in various embodiments, simply associated with an oligonucleotide in the shell of oligonucleotides attached to the exterior of the nanoparticle core of the SNA, and/or the therapeutic agent is associated with the nanoparticle core of the SNA, and/or the therapeutic agent is encapsulated in the SNA. In some embodiments, the therapeutic agent is associated with the end of an oligonucleotide in the shell of oligonucleotides that is not attached to the nanoparticle core (e.g., if the oligonucleotide is attached to the nanoparticle core through its 3′ end, then the therapeutic agent is associated with the 5′ end of the oligonucleotide). Alternatively, in some embodiments, the therapeutic agent is associated with the end of an oligonucleotide in the shell of oligonucleotides that is attached to the nanoparticle core (e.g., if the oligonucleotide is attached to the nanoparticle core through its 3′ end, then the therapeutic agent is associated with the 3′ end of the oligonucleotide). In some embodiments, the therapeutic agent is covalently associated with an oligonucleotide in the shell of oligonucleotides that is attached to the exterior of the nanoparticle core of the SNA. In some embodiments, the therapeutic agent is non-covalently associated with an oligonucleotide in the shell of oligonucleotides that is attached to the exterior of the nanoparticle core of the SNA. However, it is understood that the disclosure provides SNAs wherein one or more therapeutic agents are both covalently and non-covalently associated with oligonucleotides in the shell of oligonucleotides that is attached to the exterior of the nanoparticle core of the SNA. It will also be understood that non-covalent associations include hybridization, protein binding, and/or hydrophobic interactions. In some embodiments, a therapeutic agent is administered separately from a SNA of the disclosure. Thus, in some embodiments, a therapeutic agent is administered before, after, or concurrently with a SNA of the disclosure to treat a disorder.

Therapeutic agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### EXAMPLES

With respect to the Examples below, reference to use of a “CRISPR-SNA” may indicate utilization of a Cas9 protein that does not include any GALA peptide sequences. Also with respect to the Examples below, reference to use of a “Cas9 SNA” may indicate utilization of a “fused” Cas9 protein as described herein, which comprises the following structure in order from N-terminus to C-terminus: (i) one or more GALA peptides; (ii) a gene editing protein, and (iii) a nuclear localization signal (NLS).

### Example 1

### Use of LSNAs in Gene Editing

The present disclosure provides methods for delivering gene-editing proteins into mammalian cells using spherical nucleic acids. Enzymatically active ribonucleoprotein (RNP) complexes of Streptococcus pyogenes Cas9 with tracrRNA and crRNA are synthesized, then RNPs are encapsulated in liposomes made from 95% 1,2-dioleoyl-sn-glycero-3 phosphatidylcholine (DOPC) and 5% 1,2-dipalmitoyl-sn-glycero-3-phosphoethanolamine-N-(6-azidohexanoyl) (DPPE-Azide). The liposomes are then functionalized with 5′ DBCO-modified DNA, to generate LSNAs. These particles contain enzymatically active Cas9 and are efficiently taken up by mammalian cells.

### Methods

Unless otherwise noted, all reagents were purchased from commercial sources and used as received. For oligonucleotide, crRNA and tracrRNA synthesis, all phosphoramidites and reagents were purchased from Glen Research, Co. (Sterling, VA, USA). All lipids were purchased from Avanti Polar Lipids (Alabaster, AL, USA) either in dry powder form or chloroform and used without further purification. EnGen® Cas9 NLS (Cas9), Proteinase K and Phusion PCR kits were purchased from New England Biolabs (Ipswich, MA, USA). Alexa Fluor 647 NHS ester dye (Alexa 647) was purchased from Lumiprobe Corp. (Cockneysville, MD, USA). Plasmids were purchased from AddGene (Cambridge, MA, USA. GelRed dye was purchased from Biotium Inc. (Fremont, CA, USA). All other reagents were purchased from Sigma-Aldrich (St. Louis, MO, USA). C166-GFP cells were purchased from ATCC (Manassas, VA, USA), and Opti-MEM was purchased from Life Technologies (Carlsbad, CA).

### Cas9 Labeling and Quantification

In order to track and quantify Cas9, 2 nanomoles of Cas9 was incubated with 10 nanomoles of Alexa 647 NHS Ester, in 1× HBS overnight at 4° C., generating Alexa-Cas9. To remove unreacted dye, Alexa-Cas9 was run through a NAP5 column equilibrated in 1× HBS, and eluted in 1 mL 1× HBS. 2 nanomoles unmodified Cas9 was exchanged into 1× HBS using a NAP5 column, and combined with the Alexa Cas9. The concentration of Cas9 and Alexa dye were calculated using the absorbance at 280 nm and 650 nm, respectively, and the molar ratio of Alexa dye to Cas9 was calculated. The Alexa-Cas9 was then diluted to 1 μM. A 20 μL aliquot was reserved for activity and concentration assays.

### Cas9 Ribonucleoprotein Synthesis and Concentration

Ten nanomoles crRNA and tracrRNA were generated by incubating 10 μM crRNA with 10 μM tracrRNA in 1× HBS at 95° C., for 5 minutes, and allowed to cool to room temperature for 10 minutes. Ten nanomoles of crRNA/tracrRNA complex is then mixed with 4 nanomoles of 1 μM Alexa-Cas9 and allowed to sit at room temperature for 10 minutes, to form the Cas9 ribonucleoprotein (RNP). RNPs were then concentrated in Amicon 10K spin filters for five minute stretches, then resuspending, until the retained liquid volume reaches 500 μL or less. Cas9 concentration was again quantified using the absorbance of the Alexa 647 dye. Twenty μL were set aside for activity and concentration measurements.

### Synthesis and Purification of SNAs

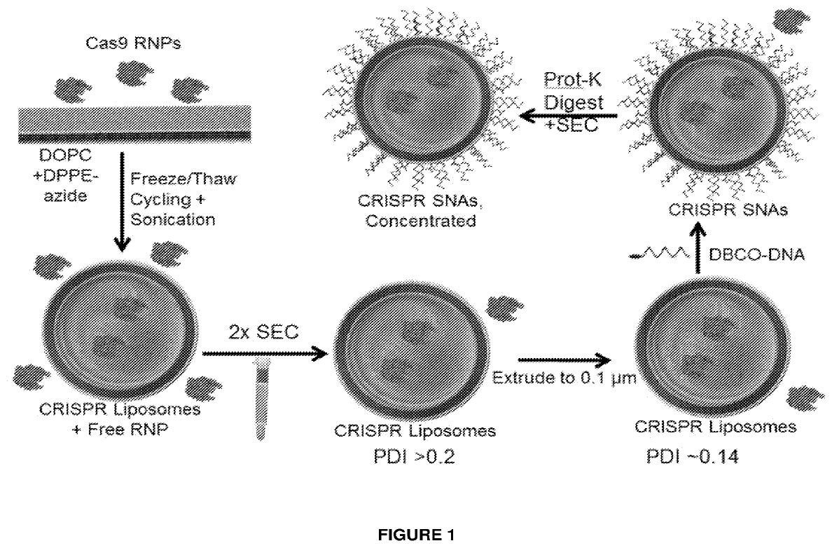

To synthesize liposomes encapsulating Cas9 RNPs, a dehydrated phospholipid film was generated by lyophilizing a mixture of 3 mg DOPC and 0.15 mg DPPE-Azide in chloroform. The lipid film was then rehydrated with 400 μL of Alexa 647-labeled ribonucleoprotein complexes (Alexa-RNPs) in 1× HBS, at a concentration of 5-8 μM. This solution was then subjected to 7 freeze/thaw cycles using liquid nitrogen and a room-temperature bath sonicator to generate single unilamellar vesicles (SUVs). The SUVs were run through a column packed with Sepharose 6B and equilibrated in 1× HBS to separate them from unencapsulated RNPs. To reduce polydispersity, the SUVs were extruded twice through 200 nm and then 100 nm membrane filters. To remove the remaining unencapsulated RNPs, SUVs were incubated for 1 hour at room temperature with proteinase K (10 U, in 500 μL 1× NEB Buffer 2+1× HBS). SUVs were separated from digested RNPs using a column packed with Superdex 200 and equilibrated in 1× HBS. To generate SNAs, the SUVs were then incubated overnight with oligonucleotides functionalized on the 5′ end with DBCO and internally with Cy3 (approximately 1 DNA per 20 phospholipids). SNAs were then separated from free oligonucleotides using a column packed with Superdex 200 and equilibrated in 1× HBS. See FIG. 1.

### Quantification of Cas9 and DNA Loading

To measure SUV concentrations, inductively coupled plasma optical emission spectrometry (ICP-OES) and a phosphorus standard were used to calculate phospholipid concentration. Liposome diameter was measured via dynamic light scattering (DLS), and the number of phospholipids per liposome were calculated using Equation 1, below. SUV concentration was calculated by dividing phospholipid concentration by the number of phospholipids per SUV.

After synthesizing SNAs (FIG. 1), the concentration of oligonucleotides was measured in a plate reader by treating SNA samples with 0.1% Tween 20 detergent (to disrupt the liposomes and disperse the oligonucleotides), and comparing Cy3 fluorescence in SNA samples to a standard curve generated from free DBCO- and Cy3-labeled oligonucleotides. The concentration of liposomes was determined with ICP-OES as above, with phosphorus concentration corrected based on the concentration of oligonucleotides and the number of phosphorus atoms per oligonucleotide.

To calculate the concentration of RNPs, a standard curve was generated from the reserved Alexa-RNP aliquot. The concentration of RNPs was determined by measuring Alexa 647 fluorescence from the liposome samples, and then plotting it on the linear regression of the Alexa-RNP standard curve in a plate reader.

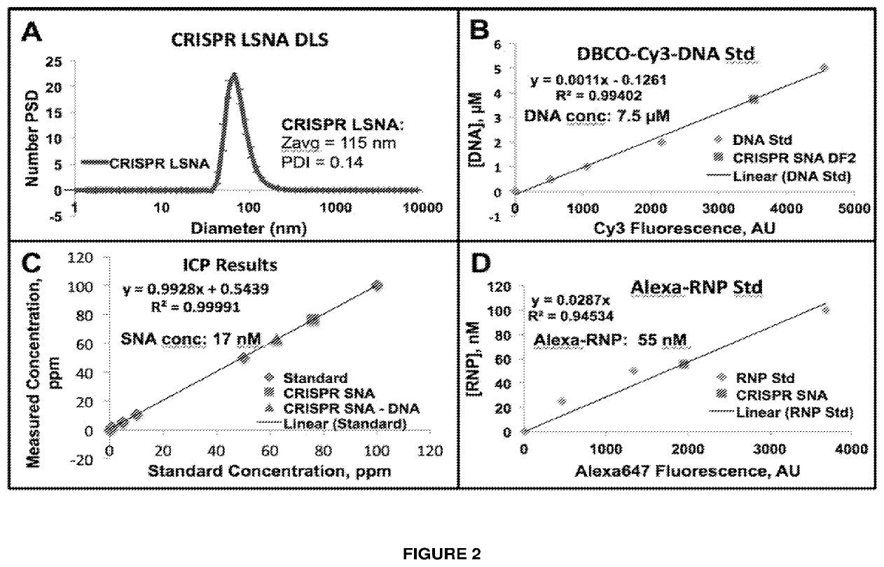

In a representative synthesis, 115 nm CRISPR SNAs were generated with approximately 450 DNA strands per particle, and encapsulated approximately 3 RNPs per liposome (FIG. 2).

### In Vitro Cas9 DNA Cleavage Assay

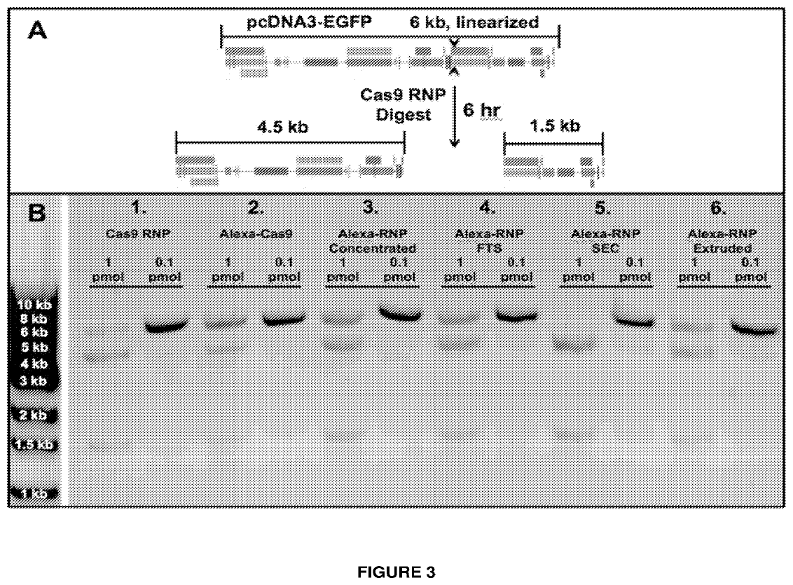

To measure Cas9 enzymatic activity, RNPs targeting the EGFP gene were synthesized and used to make CRISPR SNAs. Purified plasmid pcDNA3-EGFP was linearized by digesting with restriction enzyme Sma I. Active RNPs incubated with the linearized plasmid cleave it into a 2 kb and a 4 kb fragment, which can be seen on a 1% agarose electrophoresis gel run in TBE buffer for 30 minutes. To verify that RNPs do not degrade or lose activity during synthesis of the CRISPR SNAs, 200 nanograms linearized plasmids were incubated with the 1 pmol and 0.1 pmol Alexa RNP immediately after making them, after freeze/thaw cycling, after size exclusion, and after extrusion. The RNPs did not lose activity at these steps (FIG. 3).

### RNPs Remain Active Throughout SNA Synthesis Procedure—Protease Stability Studies

To verify that RNPs are encapsulated inside SNAs, clean CRISPR SNAs were incubated with proteinase K in NEB's restriction enzyme buffer 2 for 1 hour at room temperature. As a control, Alexa-RNPs were mixed with empty SNAs and incubated with proteinase K. The incubated samples were then eluted in 200 μL fractions through a Superdex 200 size exclusion column equilibrated in 1× HBS. These fractions were then imaged in a fluorescent gel scanner for Cy3 and Alexa Fluor 647 fluorescence. Whereas the encapsulated RNPs co-eluted with SNAs after proteinase K digestion, RNPs incubated with empty SNAs were digested, and RNP-associated Alexa fluorescence therefore eluted much later than SNA-associated Cy3 fluorescence.

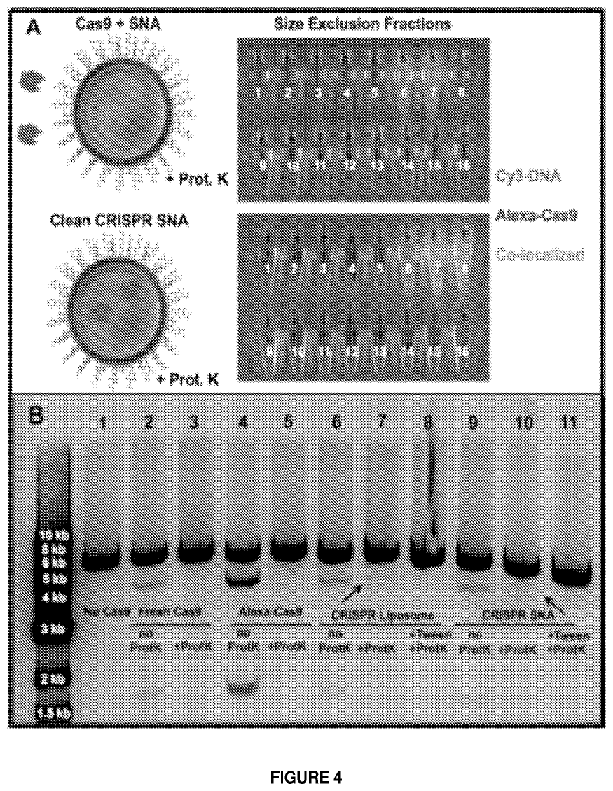

To verify that the encapsulated RNPs are still active, in vitro Cas9 DNA cleavage assays were run on several samples. The liposomes in CRISPR SNAs were disrupted with 0.1% Tween 20 detergent either before or after incubating them with proteinase K as above. In vitro DNA cleavage activity assays were performed after inactivating Proteinase K with 1 mM phenylmethylsulfonyl fluoride (PMSF). For the control RNPs, Tween had no effect on activity, but proteinase K incubation abolished activity. However, CRISPR SNAs maintained their activity if Tween was added after proteinase K incubation, but showed no activity if Tween was added before proteinase K incubation (FIG. 4). This indicated that the RNPs in CRISPR SNAs are both encapsulated (protected from protease digestion) and enzymatically active.

### Cell Uptake Studies

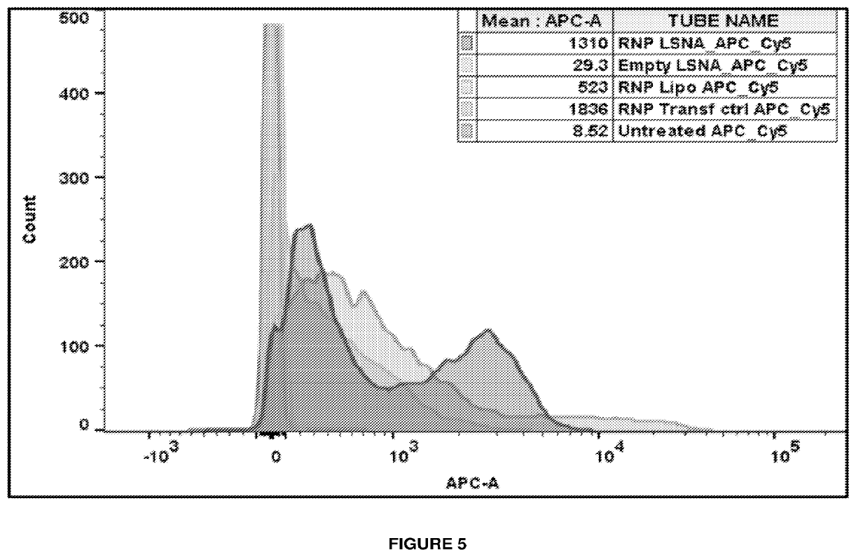

To determine if SNAs can deliver RNPs into cells, C166-GFP cells were incubated with CRISPR SNAs, empty SNAs, RNPs encapsulated in bare liposomes, and RNPs complexed with RNAiMAX transfection reagent, for 16 hours in Opti-MEM reduced serum media. Uptake of RNPs labeled with Alexa Fluor 647 was then measured via flow cytometry. Cells treated with CRISPR-SNAs had higher median fluorescence and a higher proportion of highly fluorescent (fluorescence>1000 AU) cells than those treated with RNP/RNAiMAX mixtures or RNPs encapsulated in bare liposomes, while untreated cells showed almost no fluorescence (FIG. 5). This data indicated that gene-editing enzymes encapsulated in liposomal SNAs are actively taken up into mammalian cells.

### Example 2

This example details the synthesis of a CRISPR/Cas9 ProSNA as an efficient genome editing delivery platform for a Cas9-sgRNA complex. As described herein, Cas9 serves as the nanoparticle core of ProSNAs. Surface lysine amines were reacted with small polyethylene glycol polymers with an azide and an amine-reactive N-hydroxy succinimide moiety at opposing termini. The covalently attached azides were then reacted with DNA strands containing the strained cyclooctyne, dibenzocyclooctyne (DBCO) at the 5′-terminus. The sequence used here (dGGT)₁₀ was chosen based on previous work that showed enhanced cellular uptake of SNAs with G-rich shells relative to poly dT shells. The three-dimension oligonucleotide shell creates a steric and electrostatic barrier to stabilize Cas9 proteins and renders them functional with respect to cellular entry. This strategy allows facile generation of genome editing tool with outstanding biocompatibility and cell uptake performance, and excellent genome editing activity of approximately 42.5% in human cell lines. Our findings demonstrate that the Cas9 ProSNA has attractive perspectives in the genome editing and gene silencing.

### Materials

LB broth with agar (Cat. No. L2897-250G) and LB broth were purchased from Sigma. Isopropyl β-D-1-thiogalactopyranoside (Cat. No. DSI5600) were purchased from dot scientific inc. Phosphate-buffered saline (PBS, pH 7.4) was purchased from Gibco Life Technologies. SA MALDI Matrix (Cat. No. 90032), Alexa Fluor 647 (Cat. No. A37573) and NHS-PEG4-Azide (Cat. No. 26130) were purchased from ThermoFisher. T7 RNA Polymerase (M0251S) was purchased from NEB. Ultrapure water (18.25 MΩ·cm, 25° C.) was used to prepare all solutions.

### Oligonucleotide Design, Synthesis and Purification

Oligonucleotides were synthesized on solid supports using reagents obtained from Glen Research and standard protocols. Products were cleaved from the solid support using 30% NH₄OH overnight at room temperature, and purified using reverse-phase HPLC with a gradient of 0 to 75% acetonitrile in triethylammonium acetate buffer over 45 minutes. After HPLC purification, the final dimethoxytrityl group was removed in 20% acetic acid for 2 hours, followed by an extraction in ethylacetate. The masses of the oligonucleotides were confirmed using matrix-assisted laser desorption ionization mass spectrometry using 3-hydroxypicolinic acid as a matrix. sgRNA was synthesized with NEB T7 Transcription Kit according to the manual.

| | |
|---|---|
| T₄(GGT)₁₀ | DBCO-dT-TTT(TGG)₁₀ |
| | (SEQ ID NO: 5) |
| DNase I-sgRNA | CATCAAGCTGACTAGATAATCTAGC |
| | TGATCGTGGACCGATCATACGTATA |
| | ATGCCGTAAGATCACGGGTCGCAGC |
| | ACAGCTCGCGGTCCAGTAGTGATCG |
| | ACACTGCTCGATCCGCTCGCACCGC |
| | TAGCTAATACGACTCACTATAGGCC |
| | CAGACTGAGCACGTGAGTTTTAGAG |
| | CTAGAAATAGCAAGTTAAAATAAGG |
| | CTAGTCCGTTATCAACTTGAAAAAG |
| | TGGCACCGAGTCGGTGCTTTTAAAA |
| | AGCTTGGATCGACGA |
| | (SEQ ID NO: 2) |
| GRIN2B-sgRNA | CATCAAGCTGACTAGATAATCTAGC |
| | TGATCGTGGACCGATCATACGTATA |
| | ATGCCGTAAGATCACGGGTCGCAGC |
| | ACAGCTCGCGGTCCAGTAGTGATCG |
| | ACACTGCTCGATCCGCTCGCACCGC |
| | TAGCGAAATTAATACGACTCACTAT |
| | AGGTCAACTCGTCGACTCCCTGCAG |
| | TCATAGTTCCCCTGAGAAATCAGGG |
| | TTACTATGATAAGGGCTTTCTGCCT |
| | AAGGCAGACTGACCCGCGGCGTTGG |
| | GGATCGCCTGTCGCCCGCTTTTGGG |
| | GGGCATTCCCCATCCTT |
| | (SEQ ID NO: 3) |
| GFP-sgRNA | CATCAAGCTGACTAGATAATCTAGC |
| | TGATCGTGGACCGATCATACGTATA |
| | ATGCCGTAAGATCACGGGTCGCAGC |
| | ACAGCTCGCGGTCCAGTAGTGATCG |
| | ACACTGCTCGATCCGCTCGCACCGC |
| | TAGCGAAATTAATACGACTCACTAT |
| | AGGTATGGCTAGCATGACTGGTGGG |
| | TCATAGTTCCCCTGAGAAATCAGGG |
| | TTACTATGATAAGGGCTTTCTGCCT |
| | AAGGCAGACTGACCCGCGGCGTTGG |
| | GGATCGCCTGTCGCCCGCTTTTGGG |
| | GGGCATTCCCCATCCTT |
| | (SEQ ID NO: 4) |

### Synthesis and Characterization of Cas9 SNA

Cas9 expression and purification. The Cas9 plasmid (#87703) was transformed into One Shot®BL21 (DE3) Chemically Competent E. coli (Thermo Fisher) by electricity shock, and cells were grown overnight on LB Agar plates with 100 μg/mL Ampicillin. Single colonies were picked, and 7 mL cultures were grown overnight at 37° C. in LB broth. These cultures were added to 750 mL of 2×YBT Broth and 100 μg/mL Ampicillin, and cells were grown at 37° C., to an optical density of 0.6-0.9, then induced with 1 mM Isopropyl β-D-1-thiogalactopyranoside overnight at 17° C. Cells were spun down (6000 g, 15 minutes) and resuspended in 100 mL of 1× PBS, then lysed using a high-pressure homogenizer. The cell lysate was clarified by centrifugation at 30000 g for 30 minutes and loaded onto a Bio-Scale™ Mini Profinity™ IMAC Cartridge (Bio-Rad). The column was washed with 100 mL of 1× PBS, then eluted in the same buffer with 250 mM imidazole. The eluted fraction was further purified by dialysis.

Reaction of Surface-Accessible Cysteines with Alexa Fluor 647 (AF647). The Cas9 protein was dissolved in 1× phosphate-buffered saline (1× PBS; Thermo Fisher Scientific). Then, 10 equivalents of Alexa Fluor 647-C2-maleimide (Thermo Fisher Scientific), dissolved in DMSO, were added to approximately 10 μM Cas9 in 1500 μL 1× PBS and the reaction was shaken (900 rpm) overnight. Unconjugated Alexa Fluor 647 was removed by repeated rounds of centrifugation using a 100 kDa filter until the filtrate did not have a detectable absorbance at 650 nm by UV-Vis. The number of Alexa Fluor 647 modifications per protein was calculated based on UV-Vis spectroscopy.

Reaction of Surface-Accessible Lysines with NHS-PEG4-Azide. 50 equivalents of NHS-PEG₄-azide crosslinker (Thermo Fisher Scientific), dissolved in anhydrous DMSO at a concentration of 100 mM, were added to approximately 45 μM Cas9-AF647 in 550 μL 1× PBS. The reaction was shaken (900 rpm) overnight at 25° C. Unconjugated linker was removed by 10 rounds of centrifugation using a 100 kDa filter. The number of azide modifications was assessed by MALDI-MS using sinapinic acid (Thermo Fisher Scientific) as a matrix in a Bruker AutoFlex-III.

DNA conjugation. DNA conjugation was carried out immediately after purification. 350 equivalents of DBCO-dT terminated DNA strands were first lyophilized, then 10 μM Cas9-AF647-azide in 450 μL 1× PBS was added to rehydrate the DNA. This solution was incubated for 72 hours at 25° C. with shaking (900 rpm). Unreacted DNA strands were removed by successive rounds of centrifugation in a 100 kDa filter until the filtrate did not have a detectable absorbance at 260 nm. Typically, complete removal of DNA required 30-40 washing steps. The number of DNA strands per protein was calculated based on UV-Vis spectroscopy and MALDI-MS.

Binding and cleavage activities of Cas9 SNA-sgRNA complexes. To assemble Cas9 SNA-sgRNA complexes, purified Cas9 SNA and sgRNA targeting a non-coding region within human genome were incubated in 1× NEBuffer 4.1 for 30 minutes at 37° C. with a concentration of 30 nM and 60 nM, respectively. Afterwards, Cy5-labeled DNA bearing target sequence was added to give a concentration of 150 nM, and the mixture was further incubated for 30 minutes under the same condition. Before analysis using 6% native PAGE gel, 10 μL of reaction was mixed with 2 μL 6× native loading buffer to investigate cleavage activities.

In Vitro Investigations on Cas9 SNA. Cell lines HaCaT (human keratinocyte cell line), EGFP expressing HEK293 (Human embryonal kidney cells, HEK293/EGFP) were purchased from American Type Culture Collection. Cells were cultured in Dulbecco's Modified Eagle Medium (DMEM) media supplemented with 10% fetal bovine serum and 1% penicillin-streptomycin at 37° C., in a humidified 5% CO₂ atmosphere.

Cell uptake in HaCat cells. HaCaT cells were seeded in flow cytometry tube (0.7×10⁵, 0.5 mL), and were cultured overnight in DMEM with 10% FBS. Afterwards, the culture medium was replaced with 450 μL of OPTI-MEM, and 50 μL Cas9 SNA was added and mixed to give final concentrations of 20 nM for different time intervals (0.5 hour, 1 hour, 2 hours, 4 hours, 6 hours, 8 hours). Post-treatment, cells were washed with 1× PBS, 300 μL trypsinized (Gibco), 300 μL 1× PBS was added to wash, 300 G 5 minutes, then the cells were resuspended in 1 mL of PBS. The cells were counted the density adjusted with PBS to 1×10⁶ cells in a 1 mL volume. 1 μL of the lived and dead dye was added to 1 mL of the cell suspension and mixed well; lived and dead stained 0.5 hour, incubated at room temperature for 30 minutes, protected from light. Cells were washed once with 0.5 mL of PBS and then fixed in 150 μL 4% paraformaldehyde (Thermo Fisher Scientific) for 15 minutes. Then 450 μL 1× PBS was added, washed for 3 minutes, after which 200 μL PBS was added, and then analyzed by flow cytometry using a BD LSRFortessa measuring the fluorescence (excitation 640 nm, emission 655-685 nm) of at least 30000 single-cell events per sample. Raw FCS files were gated based on forward and side scatter intensities and analyzed on FlowJo.

Cellular viability. Standard Cell Counting Kit-8 (CCK-8) assays were utilized to assess cellular viability. Briefly, cells were seeded in 96-well plates (1×10⁴ per well), and cultured in 200 μL DMEM media of 1% FBS overnight. Then 200 μL OPTI-MEM media of 2% FBS containing different concentrations (50 nM, 100 nM, 200 nM, 300 nM, 400 nM and 500 nM) of Cas9 SNA were added, followed by incubation for 24 hours. Afterwards, media was replaced with 200 μL of 10% CCK-8 in PBS. After continuous incubation for 0.5 hour at 37° C., 150 μL media was used to measure the absorbance at 450 nm using a microplate reader. Cellular viability was also evaluated by calcein-AM/PI staining.

In vitro gene silencing. HEK293 cells constantly expressing EGFP (HEK293/EGFP) were employed to assess gene silencing effects of Cas9 SNA. HEK293/EGFP cells were seeded in a 24-well plate (24 well plate, 1.3×10⁵ per well, 0.5 mL), and cultured at 37° C. overnight. The media was changed to 2% FBS in OPTI-MUM for 5 hours. After incubation with Cas9 SNA in POTI-MUM for 6 hours, targeting the coding region of the EGFP for 24 hours, cells were replaced with fresh medium and cultured for 5 days. Then cells were digested with trypsin-EDTA solution, and resuspended in 0.3 mL PBS for flow cytometry.

Surveyor assay. HEK293/EGFP cells were seeded in a 24-well plate (5×10⁴ cells per well), and cultured at 37° C. overnight. After the incubation with assembled Cas9 SNA (100 nM, targeting the human DNase I hyperactive site, human GRIN₂B site, and EGFP site for 24 hours, cells were replaced with fresh media and cultured for another 4 days. Then cells were harvested for genomic DNA extraction using a genomic DNA extraction kit. 250 ng of DNA extraction was combined with 2 μL of NEBuffer 2 (NEB) in a total volume of 19 μL and denatured, then re-annealed with thermocycling at 95° C., for 5 minutes, 95 to 85° C., at 2° C./s; 85 to 20° C., at 0.2° C./s. The re-annealed DNA was incubated with 1 μL of T7 Endonuclease I (10 U/μL, NEB) at 37° C., for 15 minutes. 10 μL of 50% glycerol was added to the T7 Endonuclease reaction and 12 μL was analyzed on PAGE gel (Bio-Rad) electrophoresed for 30 minutes at 200 V, then stained with 1× SYBR Gold (Life Technologies) for 30 minutes. Cas9-induced cleavage bands and the uncleaved band were used to calculated genome editing efficiencies using ImageJ. Targeted genome modifications were also detected by Sanger sequencing.

### Results

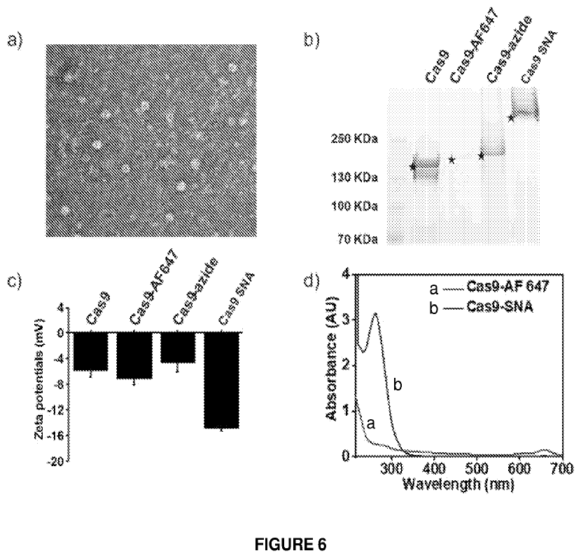

Recombinant Cas9 proteins were purified from Escherichia coli BL²¹ (DE3), and the binding/cleavage activities of Cas9-sgRNA complexes were confirmed in solution. Cas9 ProSNAs were synthesized through a previously developed method. Specifically, the Cas9 protein was tagged with Alexa Fluor 647 (AF647) to facilitate tracking in vitro and calculate the concentration of Cas9 SNA. Then, surface lysine amines were reacted with small polyethylene glycol polymers with an azide and an amine-reactive N-hydroxy succinimide moiety at opposing termini. The covalently attached azides were then reacted with DNA strands containing the strained cyclooctyne, dibenzocyclooctyne (DBCO) at the 5′ -terminus via copper-free click chemistry. The successfully synthesized Cas9 SNAs were characterized with transmission electron microscopy (TEM) with an average size of 10 nm (FIG. 6a). The purity of the synthesized protein was confirmed using SDS-PAGE gel (FIG. 6b). The gel image shows obvious molecular weight changes after each synthesized step, demonstrating the covalent attachment of oligonucleotides rather than nonspecific association with its surface. After functionalizing with DNA, the average zeta potential was changed to −15.8 mV (FIG. 6c), which enhanced the solution stability with more negative charges. The level of DNA modification was determined by comparing the difference in absorbance at 260 nm between AF Cas9 and ProSNA Cas9 (FIG. 6d). These results clearly indicated the successful DNA functionalization of Cas9.

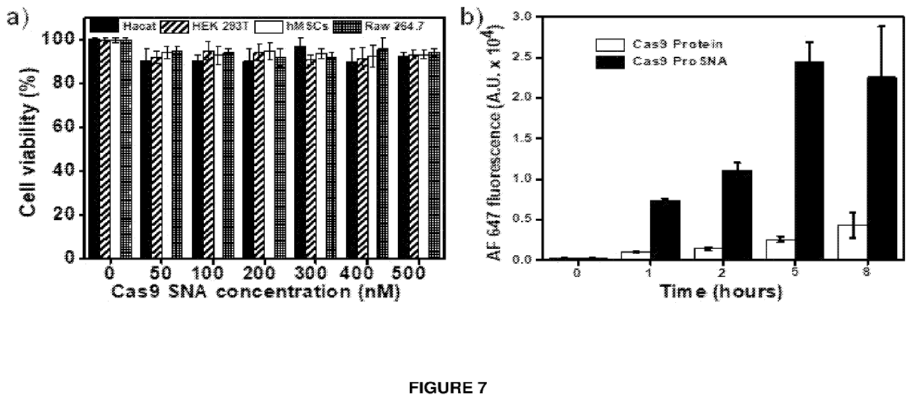

Because good biocompatibility is a prerequisite to biological applications, the viability of several cell lines was evaluated. The cytotoxicity of the Cas9 SNA was investigated using HaCat, HEK293T/EGFP, hMSCs, and Raw 264.7 cell lines as models (FIG. 7a). The standard CCK-8 was used to determine the cell viability. Although the concentration of the Cas9 SNA exceeded the typical concentration used in the in vitro experiments, no cytotoxicity was observed. The cytosolic delivery of Cas9 SNA was investigated by using HaCat cell line as a model. Cells were incubated with 20 nM protein for 0-8 hours, and their uptake performance was determined by flow cytometry (FIG. 7b). Compared to cells incubated with Cas9, Cas9 SNA showed an approximate 10-fold increase in cellular uptake. The enhanced cellular uptake of Cas9 SNA was ascribed to the engagement of cell-surface scavenger receptors followed by caveolae-mediated endocytosis.

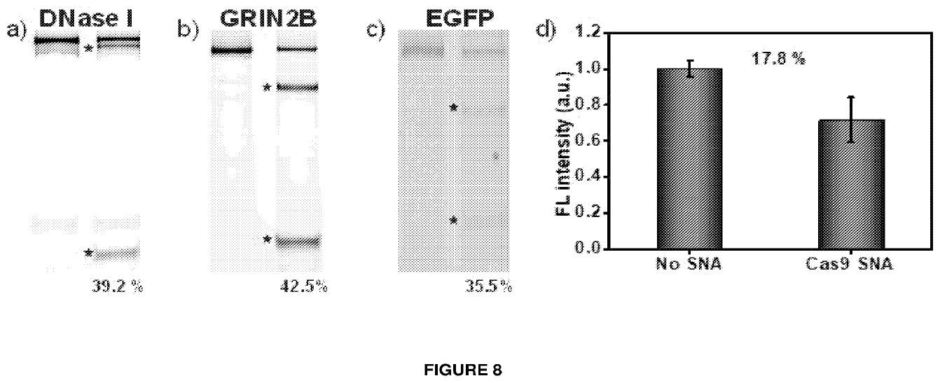

Next, the capability of Cas9 SNA in genome editing was evaluated. Cas9 SNA targeting a DNase I hypersensitive site within the human genome, namely, which is relatively safe and accessible for genome editing, was delivered to HEK 293T/EGFP cells. Surveyor assays revealed an indel frequency of 39.2% (FIG. 8a). Subsequently, Cas9 SNA targeting a site (namely GRIN2B) in gene GRIN2B related to rare neurodevelopmental disorders, was also determined, resulting in an indel frequency of 42.5% (FIG. 8b). The capability of Cas9 SNA in gene silencing was also evaluated, using a sgRNA targeting the coding region of enhanced green fluorescent protein (EGFP). The corresponding indel and EGFP silencing efficiencies were 35.5% (FIG. 8c). The gene silencing performance was also confirmed with the EGFP fluorescence change by flow cytometry, with an efficiency of 17.8%. These results demonstrated that the Cas9 SNA achieved very high editing efficiencies.

In conclusion, a CRISPR/Cas9 SNA has been established for efficient genome editing and gene silencing. The Cas9 SNA effectively entered cells by scavenger receptors pathways. Furthermore, in vitro studies demonstrated that Cas9 SNA resulted in efficient genome editing and gene silencing with good biocompatibility. This simple and versatile cytosolic delivery approach can be extended to gene therapy biomedical applications, and its superior biocompatibility opens new avenues in gene therapy and personalized medicine.

### Example 3

This example describes additional experiments using a CRISPR/Cas9 ProSNA.

### Design, Expression, and Purification of Cas9

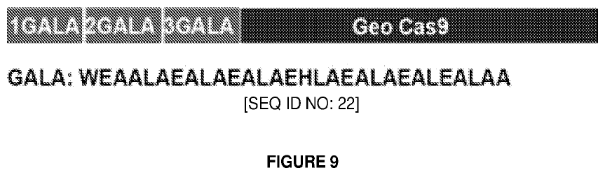

Construction of Cas9 expression vectors. pET-MBP-NLS-Geo_st expression vector (Addgene Plasmid #87703 (Harrington et al., Nat Commun. 2017 Nov. 10; 8(1):1424. doi: 10.1038/s41467-017-01408-4)) was further engineered by inserting three successive GALA peptide (3GALA) at N terminus of Geo Cas9 (FIG. 9). Sequences of all used are listed in the Table 1. GALA gene sequences were bought from Integrated DNA Technologies and cloned using Golden Gate assembly (GG). pET-MBP-NLS-Geo_st vector was firstly amplified in the PCR thermocycler (ABI), followed by removal of the original plasmid template by DpnI digestion and gel purification. Subsequently, 3GALA gene sequences were subcloned by GG-assembly into the amplified vector. The constructed vector was transformed into One Shot®BL21(DE3) by electroporation, and confirmed with traditional Sanger Sequencing, giving the 3GALA Cas9 vector. Note that the C-terminus of Cas9 contained nuclear localization signals. The amino acid sequence of the fused protein (SEQ ID NO: 24) is shown below.

MKSSHHHHHHHHHHGSSMKIEEGKLVIWINGDKGYNGLAEVGKKF
EKDTGIKVTVEHPDKLEEKFPQVAATGDGPDIIFWAHDRFGGYAQ
SGLLAEITPDKAFQDKLYPFTWDAVRYNGKLIAYPIAVEALSLIY
NKDLLPNPPKTWEEIPALDKELKAKGKSALMFNLQEPYFTWPLIA
ADGGYAFKYENGKYDIKDVGVDNAGAKAGLTFLVDLIKNKHMNAD
TDYSIAEAAFNKGETAMTINGPWAWSNIDTSKVNYGVTVLPTFKG
QPSKPFVGVLSAGINAASPNKELAKEFLENYLLTDEGLEAVNKDK
PLGAVALKSYEEELAKDPRIAATMENAQKGEIMPNIPQMSAFWYA
VRTAVINAASGRQTVDEALKDAQTNSSSNNNNNNNNNNLGIEENL
YFQSMWEAALAEALAEALAEHLAEALAEALEALAAWEAALAEALA
EALAEHLAEALAEALEALAAWEAALAEALAEALAEHLAEALAEAL
EALAASGGSSGGSSGSETPGTSESATPESSGGSSGGSMRYKIGLD
IGITSVGWAVMNLDIPRIEDLGVRIFDRAENPQTGESLALPRRLA
RSARRRLRRRKHRLERIRRLVIREGILTKEELDKLFEEKHEIDVW
QLRVEALDRKLNNDELARVLLHLAKRRGFKSNRKSERSNKENSTM
LKHIEENRAILSSYRTVGEMIVKDPKFALHKRNKGENYTNTIARD
DLEREIRLIFSKQREFGNMSCTEEFENEYITIWASQRPVASKDDI
EKKVGFCTFEPKEKRAPKATYTFQSFIAWEHINKLRLISPSGARG
LTDEERRLLYEQAFQKNKITYHDIRTLLHLPDDTYFKGIVYDRGE
SRKQNENIRFLELDAYHQIRKAVDKVYGKGKSSSFLPIDFDTFGY
ALTLFKDDADIHSYLRNEYEQNGKRMPNLANKVYDNELIEELLNL
SFTKFGHLSLKALRSILPYMEQGEVYSSACERAGYTFTGPKKKQK
TMLLPNIPPIANPVVMRALTQARKVVNAIIKKYGSPVSIHIELAR
DLSQTFDERRKTKKEQDENRKKNETAIRQLMEYGLTLNPTGHDIV
KFKLWSEQNGRCAYSLQPIEIERLLEPGYVEVDHVIPYSRSLDDS
YTNKVLVLTRENREKGNRIPAEYLGVGTERWQQFETFVLTNKQFS
KKKRDRLLRLHYDENEETEFKNRNLNDTRYISRFFANFIREHLKF
AESDDKQKVYTVNGRVTAHLRSRWEFNKNREESDLHHAVDAVIVA
CTTPSDIAKVTAFYQRREQNKELAKKTEPHFPQPWPHFADELRAR
LSKHPKESIKALNLGNYDDQKLESLQPVFVSRMPKRSVTGAAHQE
TLRRYVGIDERSGKIQTVVKTKLSEIKLDASGHFPMYGKESDPRT
YEAIRQRLLEHNNDPKKAFQEPLYKPKKNGEPGPVIRTVKIIDTK
NQVIPLNDGKTVAYNSNIVRVDVFEKDGKYYCVPVYTMDIMKGIL
PNKAIEPNKPYSEWKEMTEDYTFRFSLYPNDLIRIELPREKTVKT
AAGEEINVKDVFVYYKTIDSANGGLELISHDHRFSLRGVGSRTLK
RFEKYQVDVLGNIYKVRGEKRVGLASSAHSKPGKTIRPLQSTRDP
KKKRKV (SEQ ID NO: 24)
Bolded sequence
(WEAALAEALAEALAEHLAEALAEALEALAAWEAALAEALAEALA
EHLAEALAEALEALAAWEAALAEALAEALAEHLAEALAEALEALA
A (SEQ ID NO: 26)) is the 3GALA Peptide
Underlined sequence
(MRYKIGLDIGITSVGWAVMNLDIPRIEDLGVRIFDRAENPQTGES
LALPRRLARSARRRLRRRKHRLERIRRLVIREGILTKEELDKLFE
EKHEIDVWQLRVEALDRKLNNDELARVLLHLAKRRGFKSNRKSER
SNKENSTMLKHIEENRAILSSYRTVGEMIVKDPKFALHKRNKGEN
YTNTIARDDLEREIRLIFSKQREFGNMSCTEEFENEYITIWASQR
PVASKDDIEKKVGFCTFEPKEKRAPKATYTFQSFIAWEHINKLRL
ISPSGARGLTDEERRLLYEQAFQKNKITYHDIRTLLHLPDDTYFK
GIVYDRGESRKQNENIRFLELDAYHQIRKAVDKVYGKGKSSSFLP
IDFDTFGYALTLFKDDADIHSYLRNEYEQNGKRMPNLANKVYDNE
LIEELLNLSFTKFGHLSLKALRSILPYMEQGEVYSSACERAGYTF
TGPKKKQKTMLLPNIPPIANPVVMRALTQARKVVNAIIKKYGSPV
SIHIELARDLSQTFDERRKTKKEQDENRKKNETAIRQLMEYGLTL
NPTGHDIVKFKLWSEQNGRCAYSLQPIEIERLLEPGYVEVDHVIP
YSRSLDDSYTNKVLVLTRENREKGNRIPAEYLGVGTERWQQFETF
VLTNKQFSKKKRDRLLRLHYDENEETEFKNRNLNDTRYISRFFAN
FIREHLKFAESDDKQKVYTVNGRVTAHLRSRWEFNKNREESDLHH
AVDAVIVACTTPSDIAKVTAFYQRREQNKELAKKTEPHFPQPWPH
FADELRARLSKHPKESIKALNLGNYDDQKLESLQPVFVSRMPKRS
VTGAAHQETLRRYVGIDERSGKIQTVVKTKLSEIKLDASGHFPMY
GKESDPRTYEAIRQRLLEHNNDPKKAFQEPLYKPKKNGEPGPVIR
TVKIIDTKNQVIPLNDGKTVAYNSNIVRVDVFEKDGKYYCVPVYT
MDIMKGILPNKAIEPNKPYSEWKEMTEDYTFRFSLYPNDLIRIEL
PREKTVKTAAGEEINVKDVFVYYKTIDSANGGLELISHDHRFSLR
GVGSRTLKRFEKYQVDVLGNIYKVRGEKRVGLASSAHSKPGKTIR
PLQSTRD
(SEQ ID NO: 25) is Cas9.

Bolded and italic sequence (PKKKRKV (SEQ ID NO: 23)) is the NLS.

TABLE 1
Primer design and GALA fragments.

| | |
|---|---|
| 3GALA-For | 5′-ATGCGTTATAAGATTGGCC-3′ |
| | (SEQ ID NO: 6) |
| 3GALA-Rev | 5′-CATGGATTGGAAGTACAGG-3′ |
| | (SEQ ID NO: 7) |
| 3GALA | 5′-cctgtacttccaatccatgtgggaagc |
| | tgccctggctgaagcactggctgaagcgct |
| | ggccgaacatctggcagaagcgctggcgga |
| | agcactggaagcactggcagcgtgggaagc |
| | tgccctggctgaagcactggctgaagcgct |
| | ggccgaacatctggcagaagcgctggcgga |
| | agcactggaagcactggcagcgtctggagg |
| | atctagcggaggatcctctggcagcgagac |
| | accaggaacaagcgagtcagcaacaccaga |
| | gagcagtggcggcagcagcggcggcagcat |
| | gcgttataagattggcc-3′ |
| | (SEQ ID NO: 8) |

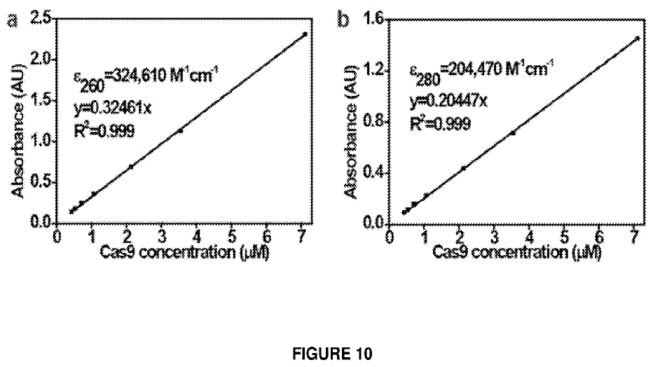

Cas9 production and purification. Recombinant Cas9 overexpression vector bearing an N-terminal 3GALA peptide was transformed into One Shot®BL21(DE3) by electricity shock, and grown overnight on LB-Ampicillin Agar plates(100 μg/mL Ampicillin). The resulting expression colony was inoculated in 7 mL (LB, 100 μg/mL Ampicillin) starter cultures which were shaken vigorously overnight at 37° C. The next day, starter culture were inoculated to 750 mL 2×YBT Broth (100 μg/mL Ampicillin) and grown at 37° C., to an optical density of 0.8, gene expression was subsequently induced with 1 mM Isopropyl β-D-1-thiogalactopyranoside followed by incubation at 17° C. overnight. Cells were harvested (6000 g, 15 minutes) and resuspended in 100 mL of lysis buffer (20 mM HEPES, pH 7.5 RT, 0.5 mM TCEP, 500 mM NaCl, 1 mM PMSF), then lysed by high-pressure homogenizer. The lysate fraction was clarified by centrifugation at 30 000 g for 30 minutes and loaded onto a 5 mL Bio-Scale™ Mini Profinity™ IMAC Cartridge (Bio-Rad) pre-equilibrated in binding buffer (20 mM HEPES, pH 7.5 RT, 500 mM NaCl). Bound protein was eluted by wash buffer (20 mM HEPES, pH 7.5, 500 mM NaCl, 250 mM imidazole). The maltose-binding protein were cleaved from eluted protein by TEV protease overnight and captured by a second MBP-affinity step. The resulting protein was loaded onto a heparin column, and eluted with a gradient from 300 to 1250 mM NaCl. The eluent fraction containing Cas9 were purified by Bio-Scale™ Mini Bio-Gel® P-6 Desalting Cartridges pre-equilibrated in storage buffer (20 mM HEPES, pH 7.5, 5% glycerol, 150 mM NaCl, 1 mM TCEP) and the concentrations were measured by a NanoDrop 8000 Spectrophotometer (Thermo Scientific) (FIG. 10). Proteins were purified at a constant temperature of 4° C., and flash frozen in liquid nitrogen and stored at −20° C.

### Oligonucleotide and sgRNA Synthesis

Oligonucleotide synthesis and purification. All phosphoramidites and DNA synthesis reagents were obtained from Glen Research. The sequences used in this work are listed in Table 2. DNA synthesis was performed by a MerMade 12 oligonucleotide synthesizer (MM12, Bio Automation Inc., Texas, USA) or an ABI 394 synthesizer on controlled pore glass (CPG) beads at 10 μmol scales. All the oligonucleotides were deprotected from the CPG beads using 30% NH₄OH overnight at room temperature. An Organomation® Multivap® Nitrogen Evaporator was then used to remove ammonia under a stream of Nitrogen. The remaining solution was filtered through a 0.2 μM filter to remove the CPG beads. The filtrate fractions were purified by reverse-phase high-performance liquid chromatography (RP-HPLC, Varian ProStar 210, Agilent Technologies Inc., Palo Alto, CA, USA) to isolate the product. An Agilent Dynamax Microsorb C4 column and a gradient of 0 to 75% B over 45 min (A=triethylammonium acetate buffer, B=acetonitrile) were used. The collected fractions were lyophilized and re-dissolved in 20% acetic acid for 2 hours and the cleaved dimethoxytrityl group was removed by ethyl acetate extraction. Matrix-assisted laser desorption/ionization time of flight mass spectrometry (MALDI-TOF MS; RapiFlex, Bruker)) was used to confirm the masses of oligonucleotides using 2′,6′-dihydroxyacetophenone and diammonium hydrogen citrate as matrix. The DNA concentration was determined by measuring the solution absorbance at λ=260 nm using UV-vis spectroscopy Cary 5000 UV-vis spectrophotometer, Varian), using the extinction coefficient of the oligonucleotide obtained from the IDT Oligo Analyzer Tool.

sgRNA design and synthesis. Synthetic dsDNA template of sgRNA bearing a consensus 5′ the T7 promoter binding site followed by the 20-bp sgRNA target sequence were in vitro transcribed using MEGAscript™ T7 Transcription Kit (ThermoFisher). Transcription was conducted in buffer containing 20 mM Tris-HCl (pH 8.0), 30 mM MgCl2, 10 mM DTT, 5 mM each NTP, 100 μg/mL T7 polymerase, RNase Inhibitor (Promega) and 100 ng DNA template. The reactions were incubated at 37° C., for approximately 18 hours. In vitro transcribed RNA was precipitated with ethanol and redissolved in water, and sgRNA concentration was finally quantified by Nano Drop 8000 Spectrophotometer (Thermo Scientific) and flash frozen in liquid nitrogen and stored at −20° C. The sequences are listed in Table 2. DNase I-sgRNA, GRIN2B-sgRNA, Grin2b-sgRNA, and EGFP-sgRNA were used to generate sgRNAs for genome editing or gene silencing at DNase I, GRIN2B, Grin2b, and EGFP sites.

TABLE 2
DNA sequences used in this Example.

| | DNA sequence |
|---|---|
| T₄(GGT)₁₀ | 5′-DBCO-dT-TTTTGGTGGTGGTGGTGGT |
| | GGTGGTGGTG GTGG-3′ (SEQ |
| | ID NO: 5) |
| DNase I-For | 5′-Cy3-CTTGTAGCTACGCCTGTGATGGG |
| | CT-3′ (SEQ ID NO: 9) |
| DNase I-Rev | 5′-Cy3-TGAGGCTGGCCCCTTCCAGG-3′ |
| | (SEQ ID NO: 10) |
| GRIN2B-For | 5′-Cy3-TGAAATCGAGGATCTGGGCGATG |
| | GC-3′ (SEQ ID NO: 11) |
| GRIN2B-Rev | 5′-Cy3-CAGGAGGGCCAGGAGATTTGTGT |
| | ATGC-3′ (SEQ ID NO: 12) |
| Grin2b-For | 5′-Cy3-CCTTTTTACCTTATCTGCCATTA |
| | TC-3′ (SEQ ID NO: 13) |
| Grin2b-Rev | 5′-Cy3-CAGACACTTCAAGGATGCGTTC |
| | C-3′ (SEQ ID NO: 14) |
| GFP-For | 5′-Cy3-ACGTAAACGGCCACAAGTTC-3′ |
| | (SEQ ID NO: 15) |
| GFP-Rev | 5′-Cy3-TGCTCAGGTAGTGGTTGTCG-3′ |
| | (SEQ ID NO: 16) |
| DNase I-sgRNA | 5′-catcaagctgactagataatctagctg |
| | atcgtggaccgatcatacgtataatgccgt |
| | aagatcacgggtcgcagcacagctcgcggt |
| | cCagtagtgatcgacactgctcgatccgct |
| | cgcaccgctagctaatacgactcactatag |
| | gcccagactgagcacgtgagttttagagct |
| | agaaatagcaagttaaaataaggctagtcc |
| | gttatcaacttgaaaaagtggcaccgagtc |
| | ggtgcttttaaaaagcttggatcgacga- |
| | 3′ (SEQ ID NO: 2) |
| GRIN2B-sgRNA | 5′-catcaagctgactagataatctagctg |
| | atcgtggaccgatcatacgtataatgccgt |
| | aagatcacgggtcgcagcacagctcgcggt |
| | ccagtagtgatcgacactgctcgatccgct |
| | cgcaccgctagcgaaattaatacgactcac |
| | tataggtcaactcgtcgactccctgcagtc |
| | atagttcccctgagaaatcagggttactat |
| | gataagggctttctgcctaaggcagactga |
| | cccgcggcgttggggatcgcctgtcgcccg |
| | cttttgggggcattccccatcctt-3′ |
| | (SEQ ID NO: 3) |
| Grin2b-sgRNA | 5′-catcaagctgactagataatctagctg |
| | atcgtggaccgatcatacgtataatgccgt |
| | aagatcacgggtcgcagcacagctcgcggt |
| | ccagtagtgatcgacactgctcgatccgct |
| | cgcaccgctagcgaaattaatacgactcac |
| | tataggatggcttcctggtccgtgtcagtc |
| | atagttcccctgagaaatcagggttactat |
| | gataagggctttctgcctaaggcagactga |
| | cccgcggcgttggggatcgcctgtcgcccg |
| | cttttggcgggcattccccatcctt-3′ |
| | (SEQ ID NO: 17) |
| EGFP-sgRNA | 5′-catcaagctgactagataatctagctg |
| | atcgtggaccgatcatacgtataatgccgt |
| | aagatcacgggtcgcagcacagctcgcggt |
| | ccagtagtgatcgacactgctcgatccgct |
| | cgcaccgctagcgaaattaatacgactcac |
| | tataggtatggctagcatgactggtgggtc |
| | atagttcccctgagaaatcagggttactat |
| | gataagggctttctgcctaaggcagactga |
| | cccgcggcgttggggatcgcctgtcgcccg |
| | cttttggcgggcattccccatcctt-3′ |
| | (SEQ ID NO: 4) |

DBCO: 5′-Dimethoxytrityl-5-[(6-oxo-6-(dibenzo[b,f]azacyclooct-4-yn-1-yl)-capramido-N-hex-6-yl)-3-acrylimido]-2′-deoxyUridine,3′-[(2-cyanoethyl)-(N,N-diisopropyl)]-phosphoramidite (5′-DBCO-dT-CE Phosphoramidite)
Cy3: 1-[3-(4-monomethoxytrityloxy)propyl]-1′-[3-[(2-cyanoethyl)-(N,N-diisopropyl)phosphoramidityl]propyl]-3,3,3′,3′-tetramethylindocarbocyanine chloride (Cyanine 3 Phosphoramidite)

### Synthesis and Characterization of Cas9 ProSNAs

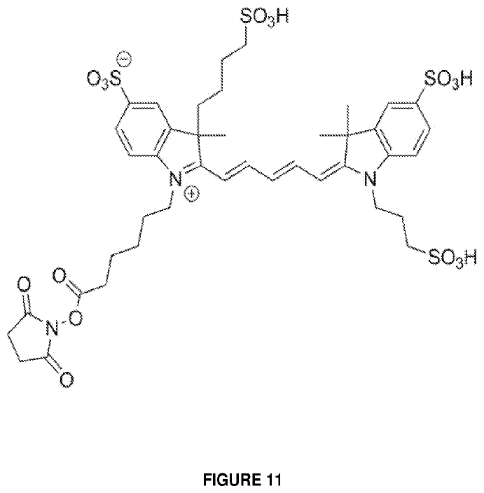

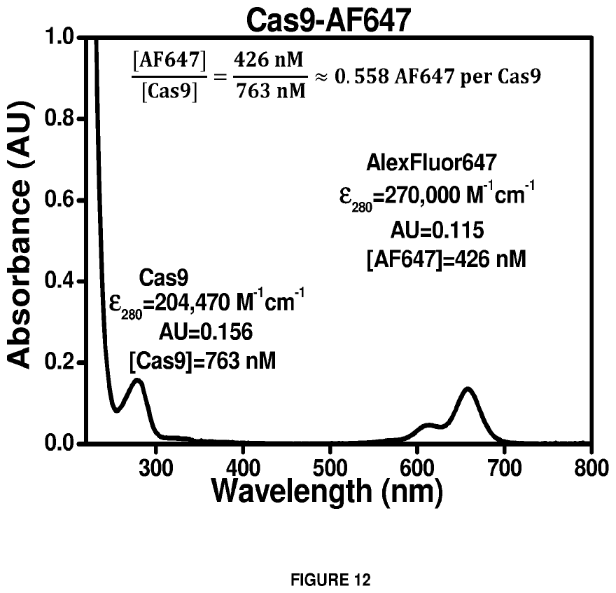

Reaction with Alexa Fluor 647 (AF647). The Cas9 protein was firstly modified with amino-active Alexa Fluor™ 647 NHS Ester (Thermo Fisher Scientific) (FIG. 11). Alexa Fluor™ 647 NHS Ester was dissolved in DMSO to obtain a 10 mM stock solution, as determined by UV-Visible absorbance spectroscopy (ε647=270,000 M⁻¹cm⁻¹). 5 equivalents excess of AF-647 were added to a solution of Cas9 protein, and the reaction was shaken at 900 rpm overnight. Excess Alexa Fluor 647 was monitored at 650 nm and removed by size exclusion chromatography on a Bio-Rad FPLC. The number of Alexa Fluor 647 modifications per protein was collected on a Cary-500 UV-vis spectrophotometer and their respective extinction coefficients (εCas9=204,470 M⁻¹cm⁻¹ at 280 nm and 324,610 M⁻¹cm⁻¹ at 260 nm; εAF-647=270,000 at 650 nm) (FIG. 12).

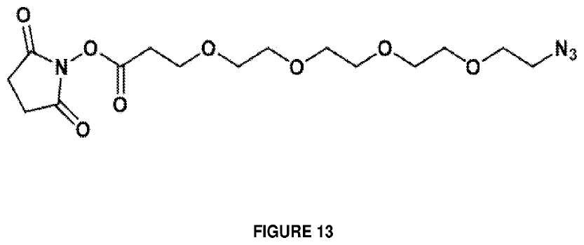

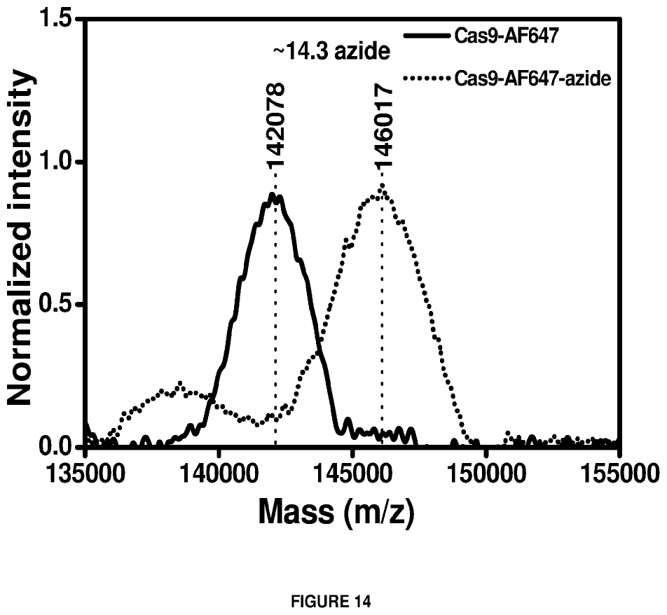

Reaction of Surface-Accessible Lysines with NHS-PEG₄-Azide. Surface amines were converted to azides by reaction with tetraethylene glycol linkers containing an N-hydroxysuccinimide (NHS) ester and an azide moiety at opposing termini (NHS-PEG₄-N₃, Thermo Scientific) (FIG. 13). 600 equivalents of NHS-PEG₄-azide crosslinker were added to a solution of Cas9-AF647. The reaction was shaken (900 rpm) overnight at 4° C. Two hours later, unconjugated azide linkers were removed by size exclusion chromatography on a Bio-Rad FPLC. The number of azide linker modifications was identified by MALDI-MS using sinapinic acid (Thermo Fisher Scientific) as a matrix in a Bruker AutoFlex-III. Each linker conjugation leads to mass increase of 275 m/z (FIG. 14).

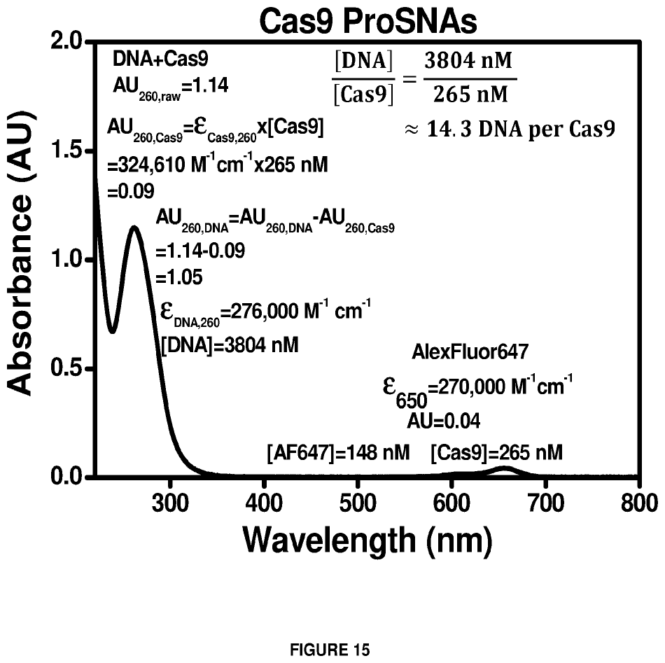

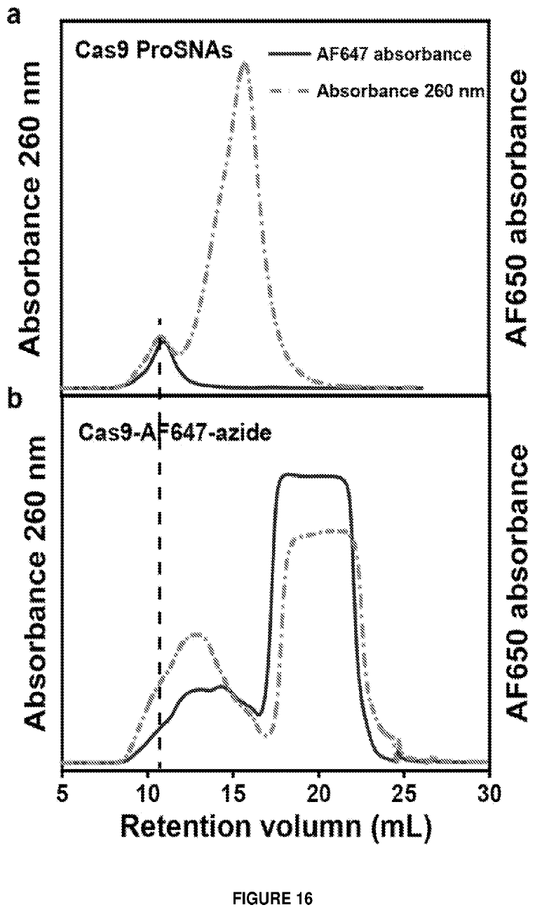

DNA conjugation. DNA conjugation was carried out immediately after Azide-functionalized Cas9 purification. 300-fold excess of DBCO terminated DNA were reacted Cas9-azide through click reaction. This reaction solution was incubated for 72 hours at 4° C. with shaking at 900 rpm. After 3 days, unreacted DNA strands were removed by size exclusion chromatography 650 on a Bio-Rad FPLC. The number of DNA per protein was determined by UV-visible absorbance spectroscopy based on the absorbance of the conjugated AlexaFluor dyes (FIG. 15). The AF-647 fluorophore was used to calculate the concentration of protein, because the absorbance overlaps at 260 nm. After subtraction of the protein absorbance, the concentration of DNA was determined based on extinction coefficients calculated using the online IDT oligo analyzer (ε260=276,000 M⁻¹cm⁻¹) (FIG. 16).

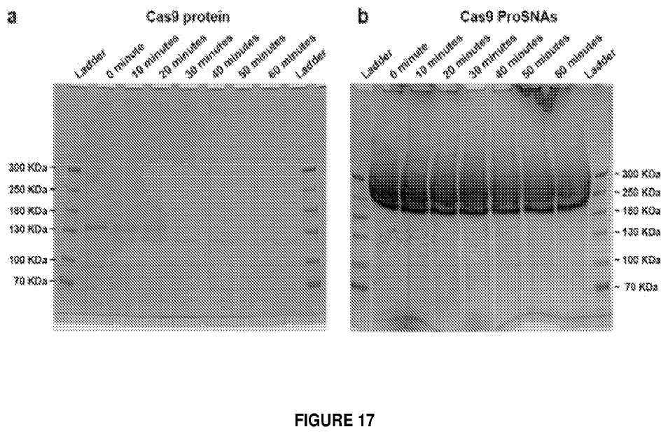

Biostability analysis of Cas9 SNA. To demonstrate whether surface conjugation of DNA could protect protein from protease degradation, both the native Cas9 protein and the Cas9 ProSNA were incubated with a trypsin (protease) and SDS-PAGE gel was performed. The reaction solution was incubated in NEB buffer 2 for over 1 hour at 37° C. It was observed that the native protein band incubated with trypsin decreased significantly after 10 minutes. However, the Cas9 ProSNA degradation was not observed suggesting that the DNA shell was able to protect the protein from substantial degradation by trypsin (FIG. 17).

### In Vitro Investigations of Cas9 ProSNAs

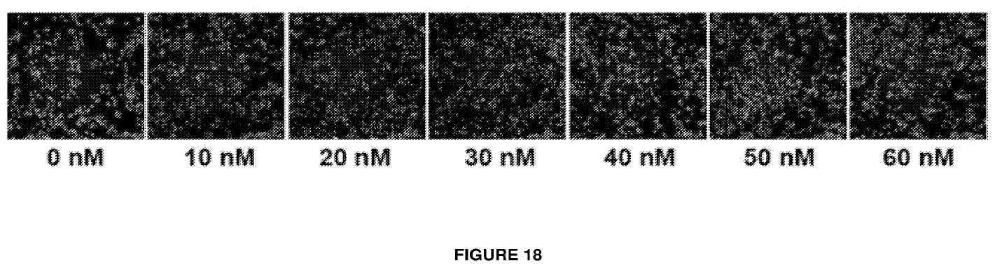

Cellular viability. Cell viability was determined with standard Cell Counting Kit-8 (CCK-8) assays. The CCK-8 reagent contains WST-8 (2-(2-methoxy-4-nitrophenyl)-3-(4-nitrophenyl)-5-(2,4-disulfophenyl)-2H-tetrazolium, monosodium salt), which can freely enter live cells. Upon cellular entry, WST-8 (a weakly fluorescent compound), is reduced by cellular dehydrogenases to orange formazan dye. Specifically, cells were seeded in 96-well cell culture plates (1×10⁴ per well), in DMEM media of 10% FBS overnight. Next, the cell culture media was replaced with 200 μL media containing different concentrations of Cas9 ProSNAs, followed by incubation for another 24 hours. Afterwards, cells were washed with 1× PBS and replaced with 10% CCK-8 in PBS. The cells were further incubated for 30 minutes. Finally, the absorbance of CCK at 450 nm was measured by BioTek Synergy H4 Hybrid Plate Reader. The experiment was performed in triplicates. Cellular viability was also evaluated by calcein-AM/PI staining. In brief, cells were seeded in 24-well plates (5×10⁴ per well), and cultured overnight, followed by the incubation in medium containing Cas9 ProSNAs for 24 hours. After that, cells were treated with 500 μL PBS containing 2 μg/mL calcein-AM and 3 μg/mL PI together. The viable or dead cells were observed by Biotek Synergy H4 Hybrid Plate Reader with 488 nm excitation for calcin-AM and 535 nm for PI (FIG. 18).

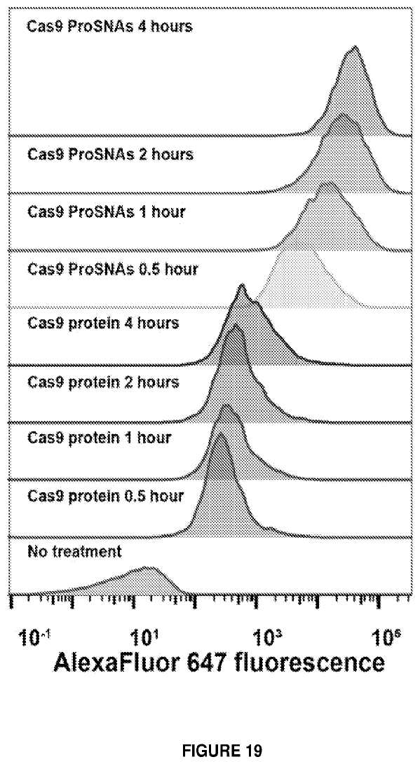

Cell uptake in HaCat cells by flow cytometry. HaCaT cells were seeded in 48 well plates (60,000 per well), and cultured overnight in DMEM with 10% fetal bovine serum (FBS) and 1% Penicillin Streptomycin. Afterwards, the culture medium was replaced with OPTI-MEM containing Cas9 ProSNAs or Cas9 AF647 to give final concentrations of 20 nM for different time intervals (0.5 hour, 1 hour, 2 hours, 4 hours, 6 hours, 8 hours). At the end of each treatment, cells were washed with 1× PBS, 300 μL trypsinized (Gibco), 300 μL 1× PBS, and centrifuged at 300 G for 5 minutes, then resuspended in 1 mL of PBS. 1 μL of the lived and dead dye was added to the cell suspension. 30 minutes later, cells were collected by centrifugation and fixed in 4% paraformaldehyde (Thermo Fisher Scientific). Flow cytometry was then conducted using a Becton Dickinson LSR II to measure the fluorescence (excitation 640 nm, emission 655-685 nm) of 10,000 single cell events per sample. Raw FCS files were gated based on forward and side scatter intensities and analyzed on FlowJo (FIG. 19).

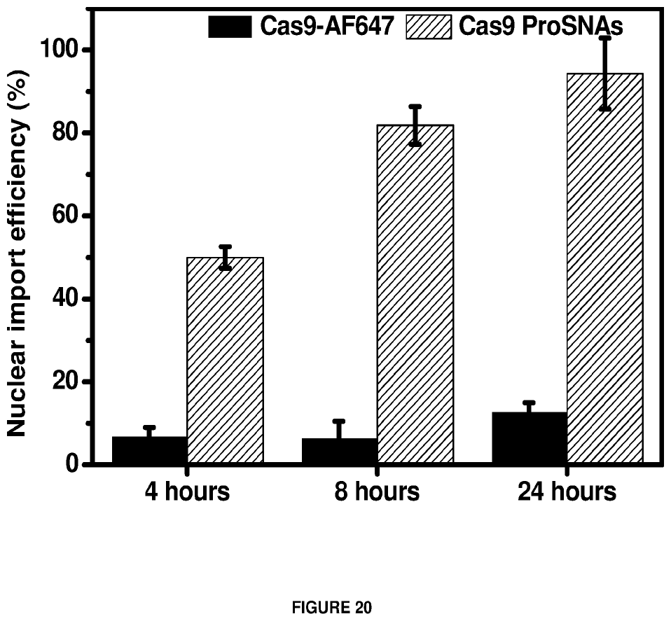

Intracellular confocal microscopy. Intracellular delivery of Cas9 ProSNAs were evaluated by the confocal laser scanning microscope (Zeiss LSM 810 microscope). To investigate endosomal escape of Cas9 ProSNAs or Cas9 AF647, HaCat cells (1×10⁴ per well) were seeded in borosilicate 8-chambered cover glass slides (Nalge Nunc International). 8 hours later, the cells were incubated with Lysosome dyes (CellLight™ Lysosomes-GFP, BacMam 2.0) at 37° C., and incubated with 500 μL of OptiMEM containing Cas9 ProSNAs or Cas9 AF647 (20 nM) for different time intervals, followed by washing with PBS and staining with nucleus dyes (Hoechst, 1 μg/mL) for 10 minutes at room temperature prior to fixing cells with 4% PFA for 10 minutes. After that, live cells were imaged by fluorescence microscopy with 405 nm for Hoechst, 488 nm for Lysosomes-GFP and 561 nm for AF 647 labelled Cas9 ProSNAs or Cas9 AF647, respectively. Nuclear import efficiency was determined by confocal microscopy as percentages of nuclei overlapped by AF647, and around 100 cells were analyzed for each sample (n=3) (FIG. 20).

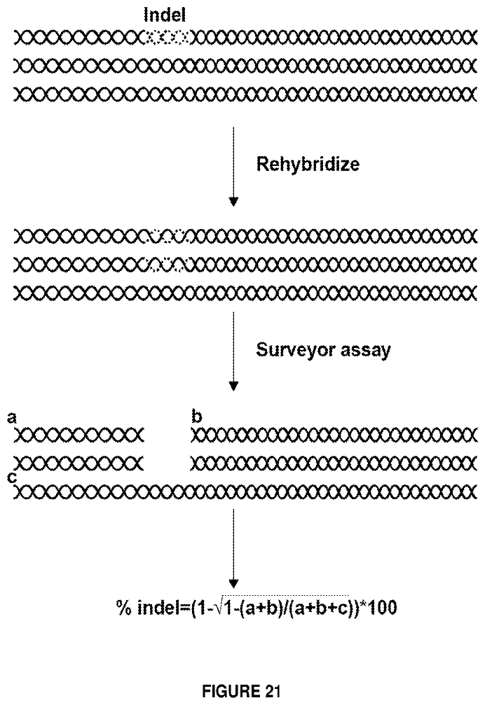

Surveyor assay (FIG. 21). HaCat, hBMSCs, RAW 264.7 and A549/EGFP cells were seeded in a 48-well plate (5'10⁴ cells per well), and cultured at 37° C. overnight. After the incubation with assembled Cas9 ProSNAs (50 nM, targeting the human DNase I hyperactive site: AGTGCTGGAGAATGGGTCACAgtggCAAA (SEQ ID NO: 18), human GRIN2B site: AGTCATTGGCAGCTACAGGCAgagaCAAA (SEQ ID NO: 19), homologous mouse Grin2b site: ATGGCTTCCTGGTCCGTGTCAtccgCGAA (SEQ ID NO: 20), and EGFP site: ACGACTTCTTCAAGTCCGCCAtgccCGAA (SEQ ID NO: 21) (underlining indicates the genome editing target)) in OPTIMEM for 4 hours, cells were replaced with fresh media and cultured for another 3 days. Then cells were harvested for the genomic DNA extraction using Quick Extraction Solution (Epicentre), followed by amplifying by PCR. For the idle assay, 250 ng of DNA extraction was combined with 2 μL of NEBuffer 2 (NEB) in a total volume of 19 μL and denatured, then re-annealed with thermocycling at 95° C., for 5 minutes, 95 to 85° C., at 2° C./s; 85 to 20° C., at 0.2° C./s. The re-annealed DNA was incubated with 1 μL of T7 Endonuclease I (10 U/μL, NEB) at 37° C., for 15 minutes. 10 μL of 50% glycerol was added to the T7 Endonuclease reaction and 12 μL was analyzed on PAGE gel (Bio-Rad) electrophoresed for 30 minutes at 200 V. Cas9-induced cleavage bands and the uncleaved band were used to calculated genome editing efficiencies using ImageJ.

Lipofectamine CRISPRMAX Cas9 transfection. The Lipofectamine CRISPRMAX transfection reagent was employed for transfecting Cas9-sgRNA complex into cells according to the provided transfection protocol. Briefly, 1 μL Cas9 Plus reagent was added to 25 μL Opti-MEM medium containing Cas9 protein (500 nM) and sgRNA (1 μM), followed by incubating at room temperature for 5 minutes (Tube1). Furthermore, 1.5 μL lipofectamine CRISPRMAX reagent was added into 25 μL Opti-MEM medium and further incubated for 5 minutes at room temperature (Tube2). After that, the Cas9-sgRNA Plus mixture from Tube1 was mixed with the lipofectamine CRISPRMAX solution from Tube2, followed by an incubation for 10 minutes at room temperature. Subsequently, 50 μL of the prepared Cas9-sgRNA transfection complex was dropped into each well (48-well plate, 5×10⁴ per well).

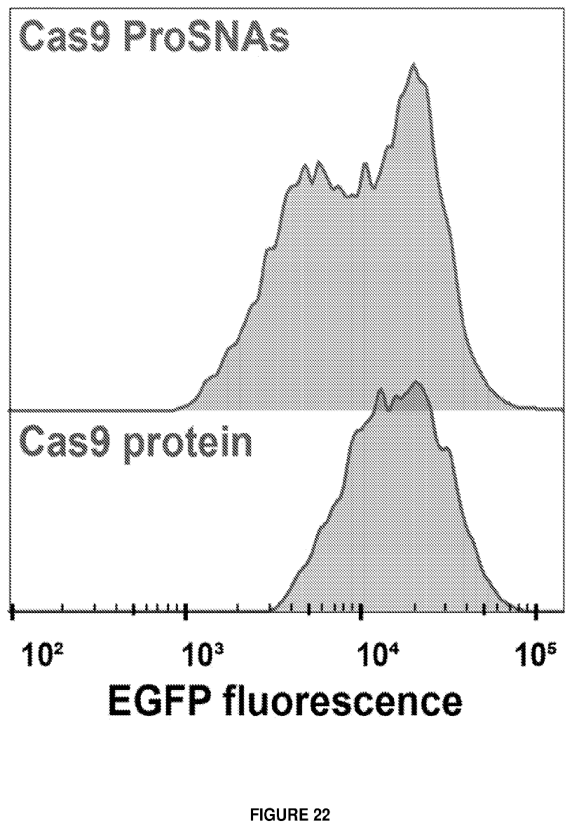

In vitro gene silencing. HEK293T cells constantly expressing EGFP (HEK293T/EGFP) were employed to assess gene silencing effects of Cas9 ProSNAs. HEK293T/EGFP cells were seeded in a 48-well plate (5×10⁴ per well, 0.5 mL), and cultured at 37° C. overnight. Then change the medium to 2% FBS in OPTI-MUM for 5 hours. After incubation with Cas9 ProSNAs in POTI-MUM for 6 hours, targeting the coding region of the EGFP for 24 hours, cells were replaced with fresh medium and cultured for 3 days. Then cells were digested with trypsin-EDTA solution, and resuspended in 0.3 mL PBS for flow cytometry (BD FACSCANTO II, the channel of EGFP). All data were analyzed using FCS Express Flow Cytometry Data Analysis (FIG. 22).

This example showed that the Cas9 ProSNAs improved cellular internalization up to approximately 45-fold. Furthermore, the employment of d(GGT)₁₀ sequence allowed facile generation of genome editing tool with outstanding biocompatibility, protease biostability, and excellent genome editing indel efficiency of approximately 45.4%. These observations lead to the design of nucleic acid functionalized (bio)macromolecular cargo, which can be used universally so that the cell function can be specific and transient manipulated.

### Example 4

This example describes additional experiments using a CRISPR/Cas9 ProSNA.

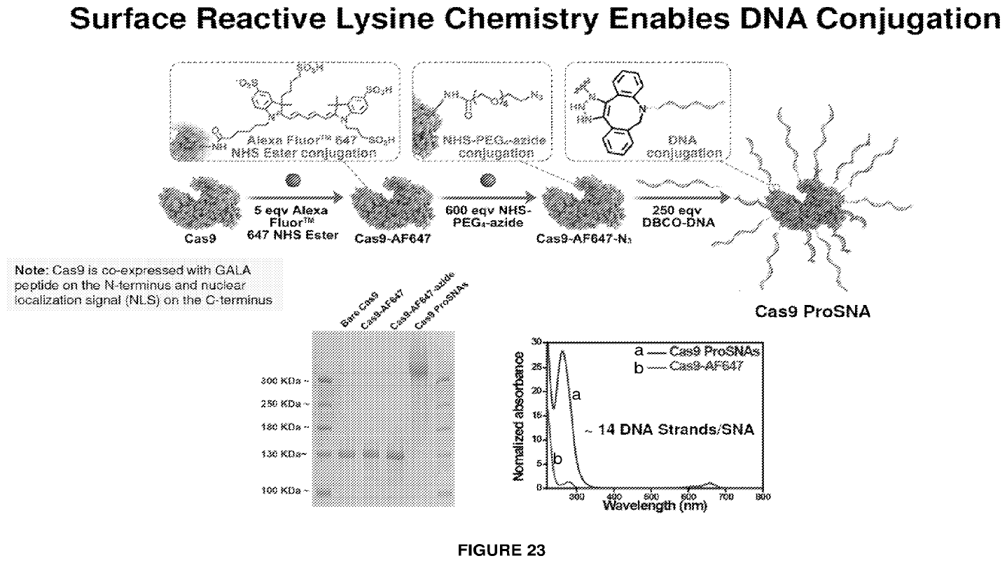

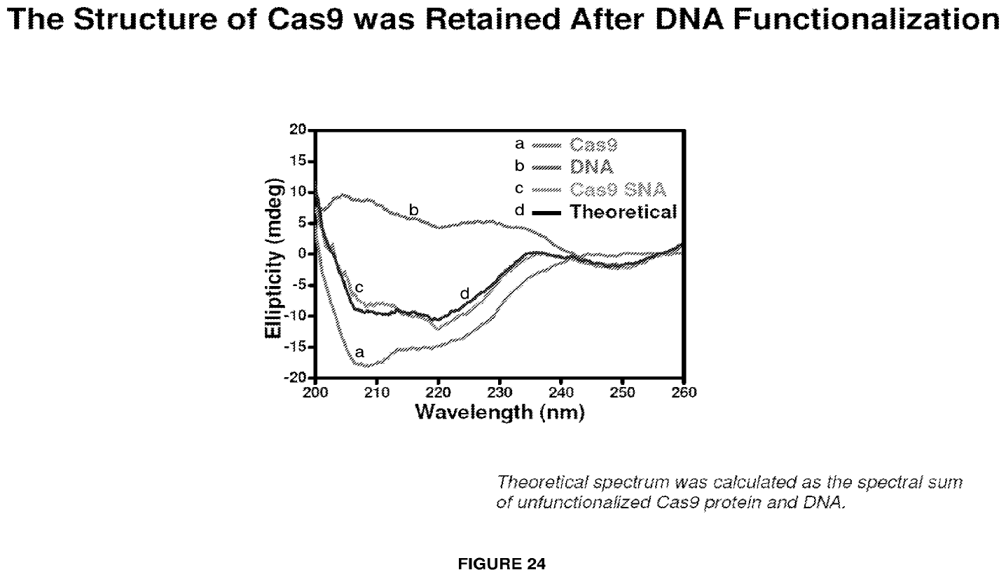

Functionalization of Alexa Fluor™ 647. The Cas9 protein was modified with amino-active Alexa Fluor™ 647 NHS Ester (AF647, Thermo Fisher Scientific). AF647 was dissolved in DMSO to obtain a 10 mM stock solution, as determined by UV-Visible spectrophotometry (ε₆₄₇=270,000 M−1cm−1). Five excess equivalents of AF647 were added to a solution of Cas9 protein, and the reaction was shaken at 900 rpm overnight. Excess AF647 was removed by size exclusion chromatography on a Bio-Rad FPLC. The spectrum of AF647 modified protein was collected on a Cary-500 UV-Visible spectrophotometer (Molecular Devices Inc, USA) and the number of modifications was calculated using their respective extinction coefficients (Cas9: ε₂₈₀=204,470 M⁻¹cm⁻¹ at 280 nm and ε₂₆₀=324.610 M⁻¹cm⁻¹ at 260 nm; AF647: ε₆₅₀=270,000 at 650 nm). (FIG. 23)

UV-Visible spectrum of AF647 fluorophore modified Cas9. Spectrum was obtained at ambient temperature on a Cary5000 spectrophotometer. Protein and AF647 concentrations were calculated from the absorbance at 280 nm and 650 nm, respectively. The AF647 fluorophore was used to calculate the concentration of protein after DNA conjugation and track cellular uptake both in the flow cytometry and confocal imaging experiments. (FIG. 23)

Reaction of surface-accessible lysine with NHS-PEG₄-Azide. Surface lysines were converted to azides by reaction with tetraethylene glycol linkers containing an N-hydroxysuccinimide (NHS) ester and an azide moiety at the opposing termini (NHS-PEG₄-N₃, Thermo Scientific). 600 equivalents of NHS-PEG₄-azide linker were added to a solution of Cas9-AF647. Two hours later, unconjugated linkers were removed by size exclusion chromatography on a Bio-Rad FPLC. The number of azide modifications was identified by MALDI-MS using sinapinic acid (Thermo Fisher Scientific) as a matrix. Each linker conjugation resulted in a mass increase of 275 m/z. (FIG. 23)

DNA conjugation. DNA conjugation reaction was carried out immediately after the purification of azide modified Cas9. 300-fold excess of DBCO terminated DNA was reacted with Cas9-AF647-azide through click reaction. This reaction solution was incubated for 3 days at 4° C. After 3 days, unreacted DNA strands were removed by size exclusion chromatography (ENrich™ SEC 650 column, Bio-Rad Inc., USA) on a Bio-Rad FPLC. The number of DNA modifications per protein was determined by UV-Visible spectrophotometry. The AF647 fluorophore was used to calculate the concentration of protein after DNA conjugation, as both absorbances overlap at 260 nm. After subtraction of the protein absorbance, the concentration of DNA was determined based on extinction coefficient obtained from Integrated DNA Technologies oligo analyzer (ε₂₆₀=276,000 M⁻¹cm⁻¹). (FIG. 23)

Determination number of DNA strands on Cas9 ProSNAs with UV-Visible spectrophotometry. Spectrum was collected on a Cary5000 spectrophotometer. Protein and DNA concentrations were calculated from the absorbance at 650 nm and 260 nm, respectively. (FIG. 23)

Circular dichroism spectroscopy. Circular Dichroism (CD) was used to characterize the intact Cas9 protein structure after DNA functionalization. All samples were buffer exchanged in PBS and CD spectra were collected on a Jasco J-1700 spectrophotometer at room temperature. Cas9 and DNA samples were prepared at the concentrations of 500 nM and 7.13 μM, respectively. A theoretical spectrum of Cas9 ProSNAs was calculated by summing the spectra of Cas9-AF647 and free DNA. The collected spectrum of Cas9 ProSNAs conformed with the calculated spectrum. (FIG. 24)

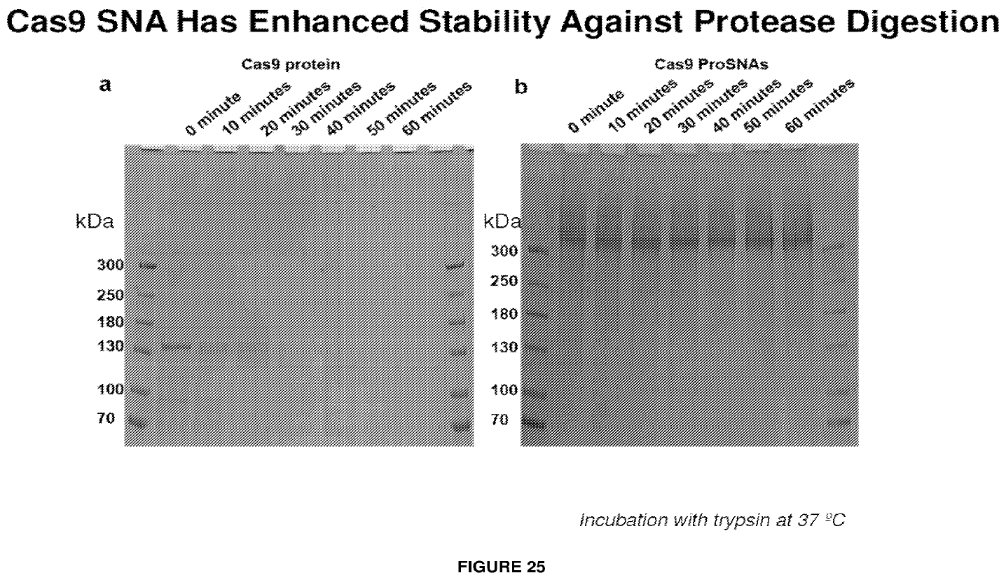

Biostability analysis of Cas9 ProSNAs. Sodium dodecyl sulphate-polyacrylamide gel electrophoresis (SDS-PAGE) was used to investigate whether surface conjugation of DNA could protect Cas9 protein from trypsin degradation. Both native Cas9 protein and Cas9 ProSNAs were incubated with trypsin at 37° C., and 30 μL of proteins were loaded for analyses across different time points. It was observed that the bands corresponding Cas9 protein diminished significantly as short as 10 minutes of incubation with trypsin. However, Cas9 ProSNAs showed almost no degradation for the time course of this study due to the DNA conjugation. (FIG. 25)

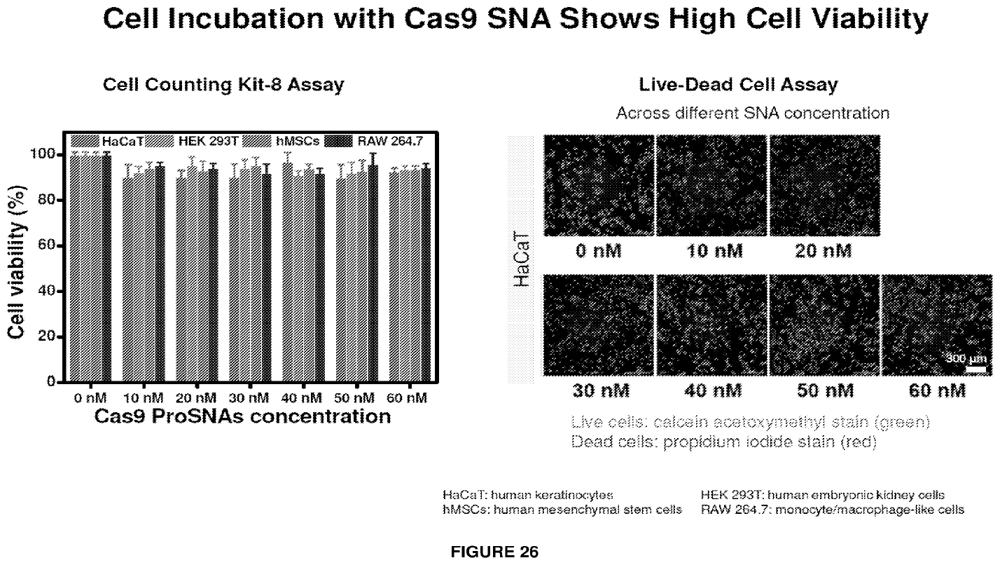

Cellular viability. Cell viability was determined with standard Cell Counting Kit-8 (CCK-8) assays. The CCK-8 reagent contained 2-(2-methoxy-4-nitrophenyl)-3-(4-nitrophenyl)-5-(2,4-disulfophenyl)-2H-tetrazolium (WST-8), which can freely enter live cells. Upon cellular entry, WST-8 was reduced by cellular dehydrogenases to orange formazan dye (absorbance at 460 nm). Specifically, cells were seeded in 96-well cell culture plates (104 per well) in DMEM media with 10% FBS overnight. Next, the cell culture media was replaced with fresh media containing different concentrations of Cas9 ProSNAs and incubated for another 24 hours. Cells were next washed with PBS and replaced with 10% CCK-8. The cells were further incubated for 30 minutes and the absorbance value at 460 nm was measured by BioTek Synergy H4 Hybrid Plate Reader. All experiments were conducted in independent triplicates. Cellular viability was also evaluated by live/dead staining. In brief, cells were seeded in 24-well plates (5×10⁴ per well) and cultured overnight, followed by incubation in cell medium containing Cas9 ProSNAs for 24 hours. After that, cells were treated with calcein acetoxymethyl (2 μg/mL) and propidium iodide (3 μg/mL) together. The viable or dead cells were observed by Biotek Synergy H4 Hybrid Plate Reader (BioRad, USA) with 488 nm excitation for calcin-AM and 535 nm for propidium iodide. (FIG. 26)

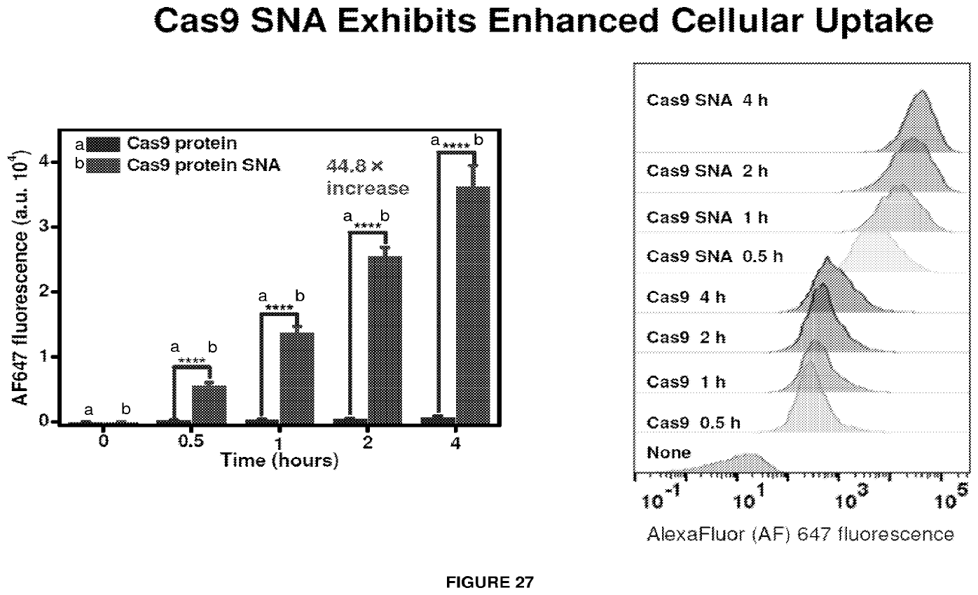

Cellular uptake in HaCat cells by flow cytometry. HaCaT cells were seeded in 48 well plates (60,000 per well) and cultured in DMEM with 10% fetal bovine serum (FBS) and 1% penicillin and streptomycin overnight. Afterwards, the cell culture media were replaced with Opti-MEM containing Cas9 ProSNAs or Cas9-AF647 to give a final concentration of 20 nM for different time intervals (0.5-hour, 1 hour, 2 hours, 4 hours, 6 hours and 8 hours). At the end of each treatment, cells were washed PBS, trypsinized (Gibco) and centrifuged at 800×g for 5 minutes and fixed with fixation buffer (BioLegend). Flow cytometry was then conducted using a Becton Dickinson LSR II to measure the fluorescence (excitation 640 nm, emission 655-685 nm) of at least 10,000 single cell events per sample. All experiments were conducted in triplicates. (FIG. 27)

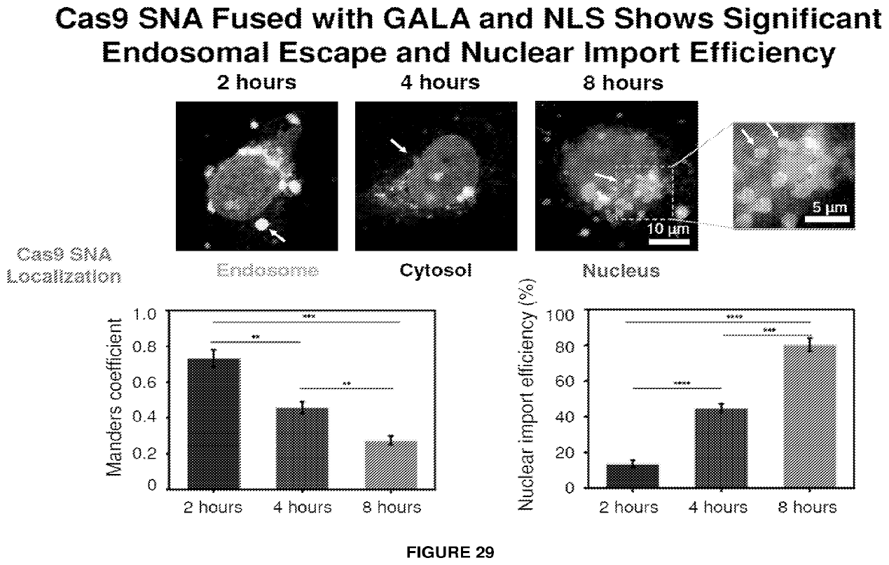

Intracellular delivery analysis by confocal microscopy. Intracellular delivery of Cas9 ProSNAs was evaluated by the confocal laser scanning microscope (Zeiss LSM 810, German). To investigate the endosomal escape of Cas9 ProSNAs or Cas9 AF647, HaCat cells (10⁴ per well) were seeded in borosilicate 8-chambered cover glass slides (Nalge Nunc International). After 8 hours, cells were incubated with endosome stain (CellLight™ Late Endosomes-GFP, BacMam 2.0) containing Cas9 ProSNAs or Cas9 AF647 (20 nM) across different time intervals. Excess proteins were washed with PBS and nucleus were stained with Hoechst (1 μg/mL) for 1 minute prior to fixing cells with 4% paraformaldehyde (Thermo Fisher Scientific) for 15 minutes. After that, cells were imaged by confocal fluorescence microscopy with 405 nm for Hoechst, 488 nm for Lysosomes-GFP and 561 nm for Cas9 ProSNAs or Cas9 AF647 at the same time. Nuclear import efficiency was determined from confocal microscopy images counting nucleuses overlapped by AF647, and around 100 cells were analyzed for each sample. (FIG. 29)

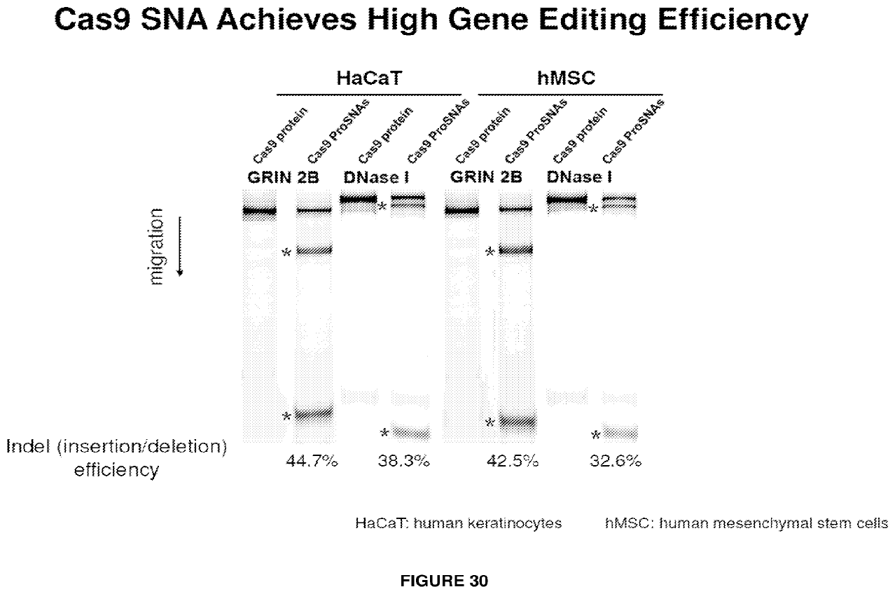

Surveyor assay to detect genome editing indel efficiency. HaCat, hBMSCs, RAW 264.7 and HEK293T/EGFP cells were seeded in 48-well plates (5×10⁴ cells per well) and cultured overnight. After transfection with assembled Cas9 ProSNAs-sgRNA complex (50 nM) in Opti-MEM for 4 hours, cells were replaced with fresh media and cultured for another 3 days. Genomic DNA was next extracted from cells using genomic DNA extraction Kit (Quick Extraction Solution, Epicentre) following the manufacturer's protocol. Briefly, cells were resuspended in QuickExtract solution and incubated at 65° C., for 15 minutes and 98° C., for 6 minutes. Then 5 μL extraction solution was amplified by PCR reaction with target region primers. For the idle formation assay, an aliquot of 5 μL of PCR product was mixed T7 endonuclease I (T7EI) buffer in a total volume of 19 μL and denatured, then re-annealed with thermocycling to allow heteroduplex formation (95° C., for 10 minutes, 95 to 85° C. ramping at −2° C./s, 85 to 20° C. ramping at −0.2° C./s. The re-annealed product was incubated with 1 μL of T7EI (10 U/μL, NEB) for 15 minutes and analyzed on 4-15% poly-acrylamide gels (BioRad). Cas9-induced cleavage bands and the uncleaved bands were visualized on Gel Doc gel imaging system (BioRad) and quantified using ImageJ densitometry analysis. Genome editing efficiency scheme was determined as shown in FIG. 21. The results are shown in FIG. 30.

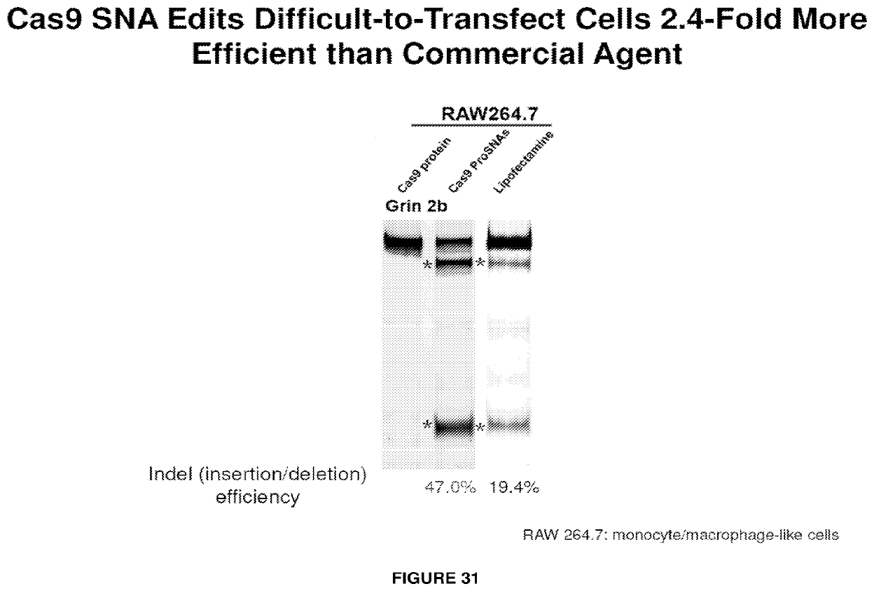

Lipofectamine CRISPRMAX Cas9-sgRNA complex transfection. The Lipofectamine™ CRISPRMAX™ transfection reagent was used to transfect Cas9-sgRNA complex into RAW 264.7 cells according to the manufactural transfection protocol. Briefly, Cas9 Plus reagent was added to medium containing Cas9 protein and sgRNA, followed by incubation at room temperature for 5 minutes to form Cas9-sgRNA complex. Then lipofectamine CRISPRMAX reagent was mixed with Opti-MEM medium and incubated for another 5 minutes. After that, the Cas9-sgRNA mixture was mixed with the lipofectamine CRISPRMAX solution, followed by incubation for 10 minutes. Subsequently, 50 μL of the prepared Cas9-sgRNA transfection complex (50 nM of final concentration) was added and mixed to the cell medium for 4 hours. After Cas9-sgRNA complex treatment, the cells were cultured in the corresponding media for 3 days. Then the cells were harvested for the subsequent Surveyor assay. Results are shown in FIG. 31.

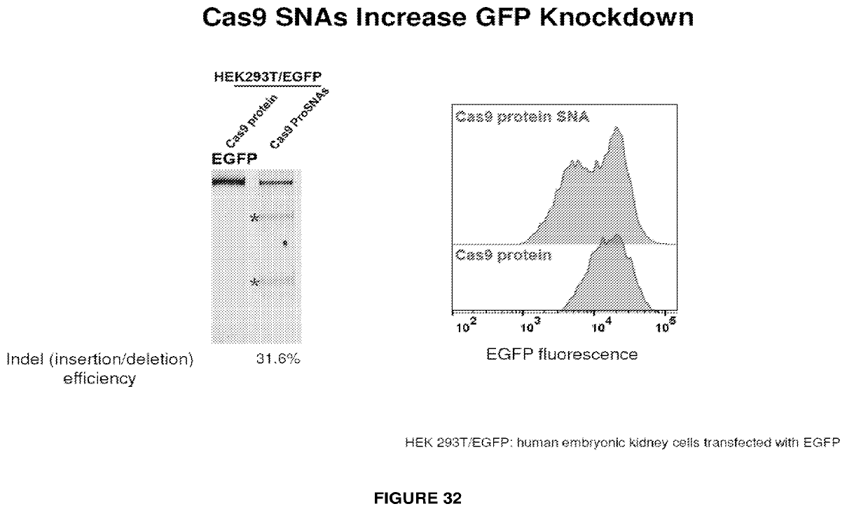

In vitro gene silencing. HEK293T cells containing EGFP gene (HEK293T/EGFP) were employed to assess gene silencing effect of Cas9 ProSNAs. HEK293T/EGFP cells were seeded in a 48-well plate (5×10⁴ per well) and cultured overnight. After incubation with Cas9 ProSNAs (50 nM) targeting the coding region of the EGFP in Opti-MEM for 4 hours, cells were replaced with fresh medium and cultured for another 3 days. Then cells were digested with trypsin-EDTA solution and resuspended in the lived and dead cell suspension solution. 30 minutes later, cells were washed with PBS and fixed for flow cytometry (Becton Dickinson LSR II, the channel of EGFP). All experiments were conducted in independent triplicates. Results are shown in FIG. 32.

What is claimed is:

1. A protein-core spherical nucleic acid (ProSNA) comprising:
(a) a protein core that comprises a gene editing protein; and
(b) a shell of oligonucleotides attached to the protein core.

2. The ProSNA of claim 1, wherein each oligonucleotide in the shell of oligonucleotides is covalently attached to the protein core.

3. The ProSNA of claim 2, wherein each oligonucleotide in the shell of oligonucleotides is attached to the protein core through a linker.

4. The ProSNA of claim 3, wherein the linker is a cleavable linker, a non-cleavable linker, a traceless linker, or a combination thereof.

5. The ProSNA of claim 3 or claim 4, wherein the linker is a carbamate alkylene dithiolate linker.

6. The ProSNA of claim 5, wherein at least one oligonucleotide in the shell of oligonucleotides comprises protein-core-NH—C(O)—O—C₂₋₅alkylene-S—S—C₂₋₇alkylene-oligonucleotide, or protein-core-NH—C(O)—O—CH₂—Ar—S—S—C₂₋₇alkylene-Oligonucleotide, and Ar comprises a meta- or para-substituted phenyl.

7. The ProSNA of claim 5, wherein at least one oligonucleotide in the shell of oligonucleotides comprises protein-core-NH—C(O)—O—C(ZA)(ZB)C₁₋₄alkylene-C(XA)(XB)—S—S—C(YA)(YB)C₁₋₆alkylene-Oligonucleotide, and ZA, ZB, XA, XB, YA, and YB are each independently H, Me, Et, or iPr.

8. The ProSNA of claim 5, wherein at least one oligonucleotide in the shell of oligonucleotides comprises protein-core-NH—C(O)—O—C(XA)(XB)—Ar—S—S—C(YA)(YB)C₂₋₆alkylene-Oligonucleotide, and XA, XB, YA, and YB are each independently H, Me, Et, or iPr.

9. The ProSNA of claim 3, wherein the linker is an amide alkylene dithiolate linker.

10. The ProSNA of claim 9, wherein at least one oligonucleotide in the shell of oligonucleotides comprises protein-core-NH—C(O)—C₂₋₅alkylene-S—S—C₂₋₇alkylene-Oligonucleotide.

11. The ProSNA of claim 9, wherein at least one oligonucleotide in the shell of oligonucleotides comprises protein-core-NH—C(O)—C₁₋₄alkylene-C(XA)(XB)—S—S—C(YA)(YB)C₁₋₆alkylene-Oligonucleotide, and XA, XB, YA and YB are each independently H, Me, Et, or iPr.

12. The ProSNA of claim 3, wherein the linker is an amide alkylene thioether linker.

13. The ProSNA of claim 12, wherein at least one oligonucleotide in the shell of oligonucleotides comprises protein-core-NH—C(O)—C₂₋₄alkylene-N-succinimidyl-S—C₂₋₆alkylene-Oligonucleotide.

14. A spherical nucleic acid (SNA) comprising:
(a) a nanoparticle core;
(b) a shell of oligonucleotides attached to the external surface of the nanoparticle core; and
(c) a gene editing protein.

15. The SNA of claim 14, wherein the nanoparticle core is a liposomal core or a lipid nanoparticle core.

16. The SNA of claim 15, wherein the lipid nanoparticle core comprises an ionizable lipid, a phospholipid, a sterol, and a lipid-polyethylene glycol (lipid-PEG) conjugate.

17. The SNA of claim 16, wherein each oligonucleotide in the shell of oligonucleotides is covalently attached to the exterior of the lipid nanoparticle core through the lipid-PEG conjugate.

18. The SNA of any one of claims 15-17, wherein the gene editing protein is encapsulated in the lipid nanoparticle core.

19. The SNA of any one of claims 15-18, wherein the ProSNA of any one of claims 1-13 is encapsulated in the lipid nanoparticle core.

20. The SNA of any one of claims 15-18, wherein a ribonucleoprotein (RNP) complex is encapsulated in the lipid nanoparticle core, the RNP comprising the gene editing protein, clustered regularly interspaced short palindromic repeat (CRISPR) RNA (crRNA), and trans-activating crRNA (tracrRNA).

21. The SNA of claim 15, wherein the liposomal core comprises a plurality of lipid groups.

22. The SNA of claim 15 or claim 21, wherein the gene editing protein is encapsulated in the liposomal core.

23. The SNA of claim 22, wherein the ProSNA of any one of claims 1-13 is encapsulated in the liposomal nanoparticle core.

24. The SNA of claim 15 or claim 21, wherein a ribonucleoprotein (RNP) complex is encapsulated in the lipid nanoparticle core, the RNP comprising the gene editing protein, CRISPR RNA (crRNA), and trans-activating crRNA (tracrRNA).

25. The SNA of any one of claims 21-24, wherein the plurality of lipid groups comprises a lipid selected from the group consisting of the phosphatidylcholine, phosphatidylglycerol, and phosphatidylethanolamine families of lipids.

26. The SNA of claim 25, wherein the lipid is selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE).

27. The SNA of any one of claims 14-26, wherein at least one oligonucleotide in the shell of oligonucleotides is attached to the exterior of the liposomal or lipid nanoparticle core through a lipid anchor group.

28. The SNA of claim 27, wherein the lipid anchor group is attached to the 5′ end or the 3′ end of the at least one oligonucleotide.

29. The SNA of claim 27 or claim 28, wherein the lipid anchor group is tocopherol or cholesterol.

30. The ProSNA of any one of claims 1-13, or the SNA of any one of claims 14-29, wherein the gene editing protein is a CRISPR-associated protein (Cas).

31. The ProSNA or SNA of claim 30, wherein the Cas is Cas9, Cas12, Cas13, or a combination thereof.

32. The ProSNA or SNA of any one of claims 1-31, wherein at least one oligonucleotide in the shell of oligonucleotides is modified on its 5′ end and/or 3′ end with dibenzocyclooctyl (DBCO).

33. The ProSNA or SNA of any one of claims 1-32, wherein the shell of oligonucleotides comprises single-stranded DNA, double-stranded DNA, single-stranded RNA, double-stranded RNA, or a combination thereof.

34. The ProSNA or SNA of any one of claims 1-33, wherein at least one oligonucleotide in the shell of oligonucleotides is a modified oligonucleotide.

35. The ProSNA or SNA of any one of claims 1-34, wherein the shell of oligonucleotides comprises about 2 to about 100 oligonucleotides.

36. The ProSNA or SNA of claim 35, wherein the shell of oligonucleotides comprises about 10 to about 80 oligonucleotides.

37. The ProSNA or SNA of claim 35, wherein the shell of oligonucleotides comprises about 5 to about 50 oligonucleotides.

38. The ProSNA or SNA of claim 35, wherein the shell of oligonucleotides comprises about 5 to about 20 oligonucleotides.

39. The ProSNA or SNA of claim 35, wherein the shell of oligonucleotides comprises about 14 oligonucleotides.

40. The ProSNA or SNA of claim 35, wherein the shell of oligonucleotides comprises about 15 oligonucleotides.

41. The ProSNA or SNA of any one of claims 1-40, wherein each oligonucleotide in the shell of oligonucleotides is about 5 to about 100 nucleotides in length.

42. The ProSNA or SNA of claim 41, wherein each oligonucleotide in the shell of oligonucleotides is about 10 to about 50 nucleotides in length.

43. The ProSNA or SNA of any one of claims 1-42, wherein one or more oligonucleotides in the shell of oligonucleotides comprises a (GGX)ₙ nucleotide sequence, wherein n is 2-20 and X is a nucleobase (A, C, T, G, or U).

44. The ProSNA or SNA of claim 43, wherein the (GGX)ₙ nucleotide sequence is on the 5′ end of the one or more oligonucleotides.

45. The ProSNA or SNA of claim 43, wherein the (GGX)ₙ nucleotide sequence is on the 3′ end of the one or more oligonucleotides.

46. The ProSNA or SNA of any one of claims 43-45, wherein the (GGX)ₙ nucleotide sequence is a (GGT)ₙ nucleotide sequence.

47. The ProSNA or SNA of any one of claims 1-46, wherein diameter of the ProSNA or SNA is about 1 nanometer (nm) to about 500 nm.

48. The SNA of any one of claims 14-47, wherein diameter of the SNA is less than or equal to about 50 nanometers.

49. The ProSNA or SNA of any one of claims 1-47, or the SNA of claim 48, wherein at least one oligonucleotide in the shell of oligonucleotides is a targeting oligonucleotide.

50. The ProSNA or SNA of any one of claims 1-47, or the SNA of claim 48, wherein the shell of oligonucleotides comprises an inhibitory oligonucleotide, an immunostimulatory oligonucleotide, a gene editor substrate DNA or RNA, or a combination thereof.

51. The ProSNA or SNA of claim 50, wherein the inhibitory oligonucleotide is an antisense oligonucleotide, small interfering RNA (siRNA), an aptamer, a short hairpin RNA (shRNA), a DNAzyme, or an aptazyme.

52. The ProSNA or SNA of claim 50, wherein the immunostimulatory oligonucleotide is a CpG-motif containing oligonucleotide, a double-stranded DNA oligonucleotide, or a single-stranded RNA oligonucleotide.

53. The ProSNA or SNA of claim 50, wherein each of the immunostimulatory oligonucleotides is a toll-like receptor (TLR) agonist.

54. The ProSNA or SNA of claim 53, wherein the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

55. A composition comprising a plurality of the protein-core spherical nucleic acids (ProSNAs) of any one of claim 1-13, 30-47, or 49-54.

56. The composition of claim 55, further comprising a guide RNA.

57. The composition of claim 55 or claim 56, wherein at least two of the ProSNAs comprise a different protein core.

58. A composition comprising a plurality of the spherical nucleic acids (SNAs) of any one of claims 14-54.

59. The composition of claim 57, wherein at least two of the SNAs comprise a different nanoparticle core.

60. A method of delivering a gene editing protein to a cell comprising contacting the cell with the ProSNA of any one of claim 1-13, 30-47, or 49-54.

61. A method of delivering a gene editing protein to a cell comprising contacting the cell with the composition of any one of claims 55-57.

62. A method of delivering a gene editing protein to a cell comprising contacting the cell with the SNA of any one of claims 14-54.

63. A method of delivering a gene editing protein to a cell comprising contacting the cell with the composition of claim 58 or claim 59.

64. A method of treating, ameliorating, and/or preventing a disorder in a subject comprising administering to the subject an effective amount of (i) the ProSNA of any one of claim 1-13, 30-47, or 49-54, (ii) the SNA of any one of claims 14-54, (iii) the composition of claims 55-59, or (iv) a combination thereof.

65. The method of claim 64, wherein the disorder is cancer, an infectious disease, an autoimmune disease, a neurodegenerative disease, an inherited disease, cardiovascular disease, or a combination thereof.

66. A fused protein comprising the following, arranged from N-terminus to C-terminus as follows:
(i) one or more GALA peptides;
(ii) a gene editing protein, and
(iii) a nuclear localization signal (NLS).

67. The fused protein of claim 66, wherein the one or more GALA peptides comprises three successive GALA peptides.

68. The fused protein of claim 66 or claim 67, wherein each of the one or more GALA peptides comprises or consists of an amino acid sequence that is at least 90% identical to the amino acid sequence as set out in SEQ ID NO: 22.

69. The fused protein of any one of claims 66-68, wherein the one or more GALA peptides comprises or consists of the amino acid sequence as set out in SEQ ID NO: 26.

70. The fused protein of any one of claims 66-69, wherein the gene editing protein is a CRISPR-associated protein (Cas).

71. The fused protein of claim 70, wherein the Cas is Cas9, Cas12, Cas13, or a combination thereof.

72. The fused protein of claim 71, wherein the Cas9 comprises or consists of an amino acid sequence that is at least 95% identical to the amino acid sequence as set out in SEQ ID NO: 1 or SEQ ID NO: 25.

73. The fused protein of claim 71 or claim 72, wherein the Cas12 comprises or consists of an amino acid sequence that is at least 95% identical to the amino acid sequence as set out in SEQ ID NO: 27.

74. The fused protein of any one of claims 71-73, wherein the Cas13 comprises or consists of an amino acid sequence that is at least 95% identical to the amino acid sequence as set out in SEQ ID NO: 29.

75. The fused protein of any one of claims 66-74, wherein the NLS comprises or consists of an amino acid sequence that is at least 95% identical to the amino acid sequence as set out in SEQ ID NO: 23 or SEQ ID NO: 28.

76. A composition comprising the fused protein of any one of claims 66-75 and a pharmaceutically acceptable carrier.

77. The ProSNA of any one of claim 1-13, 30-47, or 49-54, or the composition of any one of claims 55-57, wherein the gene editing protein is the fused protein of any one of claims 66-75.

78. The SNA of any one of claims 14-54, or the composition of claim 58 or 59, wherein the gene editing protein is the fused protein of any one of claims 66-75.

79. A method of delivering a gene editing protein to a cell comprising contacting the cell with the fused protein of any one of claims 66-75.

80. A method of delivering a gene editing protein to a cell comprising contacting the cell with the composition of claim 76.

81. A method of treating, ameliorating, and/or preventing a disorder in a subject comprising administering to the subject an effective amount of (i) the fused protein of any one of claims 66-75, (ii) the composition of claim 76, or (iii) a combination thereof.

82. The method of claim 81, wherein the disorder is cancer, an infectious disease, an autoimmune disease, a neurodegenerative disease, an inherited disease, cardiovascular disease, or a combination thereof.


SEQUENCE LISTING (29 sequences)

SEQ ID NO: 1 - protein, 1368 aa, Streptococcus pyogenes [sequence omitted]

SEQ ID NO: 2 - DNA, 265 bp, Artificial Sequence
  Synthetic
catcaagctgactagataatctagctgatcgtggaccgatcatacgtataatgccgtaagatcacgggtcgcagcacagctcgcggtccagtagtgatcgacactgctcgatccgctcgcaccgctagctaatacgactcactataggcccagactgagcacgtgagttttagagctagaaatagcaagttaaaataaggctagtccgttatcaacttgaaaaagtggcaccgagtcggtgcttttaaaaagcttggatcgacga

SEQ ID NO: 3 - DNA, 292 bp, Artificial Sequence
  Synthetic
catcaagctgactagataatctagctgatcgtggaccgatcatacgtataatgccgtaagatcacgggtcgcagcacagctcgcggtccagtagtgatcgacactgctcgatccgctcgcaccgctagcgaaattaatacgactcactataggtcaactcgtcgactccctgcagtcatagttcccctgagaaatcagggttactatgataagggctttctgcctaaggcagactgacccgcggcgttggggatcgcctgtcgcccgcttttggcgggcattccccatcctt

SEQ ID NO: 4 - DNA, 292 bp, Artificial Sequence
  Synthetic
catcaagctgactagataatctagctgatcgtggaccgatcatacgtataatgccgtaagatcacgggtcgcagcacagctcgcggtccagtagtgatcgacactgctcgatccgctcgcaccgctagcgaaattaatacgactcactataggtatggctagcatgactggtgggtcatagttcccctgagaaatcagggttactatgataagggctttctgcctaaggcagactgacccgcggcgttggggatcgcctgtcgcccgcttttggcgggcattccccatcctt

SEQ ID NO: 5 - DNA, 34 bp, Artificial Sequence
  Synthetic
  misc_feature (1)..(1): 5′-Dimethoxytrityl-5-[(6-oxo-6-(dibenzo [b,f]azacyclooct-4-yn-1-yl)-capramido-N-hex-6-yl)-3-acrylimido]- 2′-deoxyUridine,3′-[(2-cyanoethyl)-(N,N-diisopropyl)]- phosphoramidite (5′- DBCO-dT-CE Phosphoramidite) (DBCO-d)
tttttggtggtggtggtggtggtggtggtggtgg

SEQ ID NO: 6 - DNA, 19 bp, Artificial Sequence
  Synthetic
atgcgttataagattggcc

SEQ ID NO: 7 - DNA, 19 bp, Artificial Sequence
  Synthetic
catggattggaagtacagg

SEQ ID NO: 8 - DNA, 314 bp, Artificial Sequence
  Synthetic
cctgtacttccaatccatgtgggaagctgccctggctgaagcactggctgaagcgctggccgaacatctggcagaagcgctggcggaagcactggaagcactggcagcgtgggaagctgccctggctgaagcactggctgaagcgctggccgaacatctggcagaagcgctggcggaagcactggaagcactggcagcgtctggaggatctagcggaggatcctctggcagcgagacaccaggaacaagcgagtcagcaacaccagagagcagtggcggcagcagcggcggcagcatgcgttataagattggcc

SEQ ID NO: 9 - DNA, 25 bp, Artificial Sequence
  Synthetic
  misc_feature (1)..(1): 1-[3-(4-monomethoxytrityloxy)propyl]-1′- [3-[(2-cyanoethyl)-(N,N-diisopropyl)phosphoramidityl]propyl]- 3,3,3′,3′-tetramethylindocarb ocyanine chloride (Cyanine 3 Phosphoramidite) (Cy3)
cttgtagctacgcctgtgatgggct

SEQ ID NO: 10 - DNA, 20 bp, Artificial Sequence
  Synthetic
  misc_feature (1)..(1): 1-[3-(4-monomethoxytrityloxy)propyl]-1′-[3- [(2-cyanoethyl)-(N,N-diisopropyl)phosphoramidityl]propyl]-3,3,3′, 3′-tetramethylindocarb ocyanine chloride (Cyanine 3 Phosphoramidite) (Cy3)
tgaggctggccccttccagg

SEQ ID NO: 11 - DNA, 25 bp, Artificial Sequence
  Synthetic
  misc_feature (1)..(1): 1-[3-(4-monomethoxytrityloxy)propyl]-1′-[3- [(2-cyanoethyl)-(N,N-diisopropyl)phosphoramidityl]propyl]-3,3,3′, 3′-tetramethylindocarb ocyanine chloride (Cyanine 3 Phosphoramidite) (Cy3)
tgaaatcgaggatctgggcgatggc

SEQ ID NO: 12 - DNA, 27 bp, Artificial Sequence
  Synthetic
  misc_feature (1)..(1): 1-[3-(4-monomethoxytrityloxy)propyl]-1′- [3-[(2-cyanoethyl)-(N,N-diisopropyl)phosphoramidityl]propyl]-3,3, 3′,3′-tetramethylindocarb ocyanine chloride (Cyanine 3 Phosphoramidite) (Cy3)
caggagggccaggagatttgtgtatgc

SEQ ID NO: 13 - DNA, 25 bp, Artificial Sequence
  Synthetic
  misc_feature (1)..(1): 1-[3-(4-monomethoxytrityloxy)propyl]-1′- [3-[(2-cyanoethyl)-(N,N-diisopropyl)phosphoramidityl]propyl]-3,3, 3′,3′-tetramethylindocarb ocyanine chloride (Cyanine 3 Phosphoramidite) (Cy3)
cctttttaccttatctgccattatc

SEQ ID NO: 14 - DNA, 23 bp, Artificial Sequence
  Synthetic
  misc_feature (1)..(1): 1-[3-(4-monomethoxytrityloxy)propyl]-1′- [3-[(2-cyanoethyl)-(N,N-diisopropyl)phosphoramidityl]propyl]-3,3, 3′,3′-tetramethylindocarb ocyanine chloride (Cyanine 3 Phosphoramidite) (Cy3)
cagacacttcaaggatgcgttcc

SEQ ID NO: 15 - DNA, 20 bp, Artificial Sequence
  Synthetic
  misc_feature (1)..(1): 1-[3-(4-monomethoxytrityloxy)propyl]-1′- [3-[(2-cyanoethyl)-(N,N-diisopropyl)phosphoramidityl]propyl]-3,3, 3′,3′-tetramethylindocarbocyanine chloride (Cyanine 3 Phosphoramidite) (Cy3)
acgtaaacggccacaagttc

SEQ ID NO: 16 - DNA, 20 bp, Artificial Sequence
  Synthetic
  misc_feature (1)..(1): 1-[3-(4-monomethoxytrityloxy)propyl]-1′- [3-[(2-cyanoethyl)-(N,N-diisopropyl)phosphoramidityl]propyl]-3,3, 3′,3′-tetramethylindocarbocyanine chloride (Cyanine 3 Phosphoramidite) (Cy3)
tgctcaggtagtggttgtcg

SEQ ID NO: 17 - DNA, 292 bp, Artificial Sequence
  Synthetic
catcaagctgactagataatctagctgatcgtggaccgatcatacgtataatgccgtaagatcacgggtcgcagcacagctcgcggtccagtagtgatcgacactgctcgatccgctcgcaccgctagcgaaattaatacgactcactataggatggcttcctggtccgtgtcagtcatagttcccctgagaaatcagggttactatgataagggctttctgcctaaggcagactgacccgcggcgttggggatcgcctgtcgcccgcttttggcgggcattccccatcctt

SEQ ID NO: 18 - DNA, 29 bp, Artificial Sequence
  Synthetic
agtgctggagaatgggtcacagtggcaaa

SEQ ID NO: 19 - DNA, 29 bp, Artificial Sequence
  Synthetic
agtcattggcagctacaggcagagacaaa

SEQ ID NO: 20 - DNA, 29 bp, Artificial Sequence
  Synthetic
atggcttcctggtccgtgtcatccgcgaa

SEQ ID NO: 21 - DNA, 29 bp, Artificial Sequence
  Synthetic
acgacttcttcaagtccgccatgcccgaa

SEQ ID NO: 22 - protein, 30 aa, Artificial Sequence
  Synthetic
WEAALAEALAEALAEHLAEALAEALEALAA

SEQ ID NO: 23 - protein, 7 aa, Artificial Sequence
  Synthetic
  MISC_FEATURE: SV40 NLS
PKKKRKV

SEQ ID NO: 24 - protein, 1626 aa, Artificial Sequence
  Synthetic
  MISC_FEATURE: Fused Protein
MKSSHHHHHHHHHHGSSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEENLYFQSMWEAALAEALAEALAEHLAEALAEALEALAAWEAALAEALAEALAEHLAEALAEALEALAAWEAALAEALAEALAEHLAEALAEALEALAASGGSSGGSSGSETPGTSESATPESSGGSSGGSMRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRDPKKKRKV

SEQ ID NO: 25 - protein, 1087 aa, Artificial Sequence
  Synthetic
  MISC_FEATURE: Geo Cas9
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 26 - protein, 90 aa, Artificial Sequence
  Synthetic
  MISC_FEATURE: 3GALA Peptide
WEAALAEALAEALAEHLAEALAEALEALAAWEAALAEALAEALAEHLAEALAEALEALAAWEAALAEALAEALAEHLAEALAEALEALAA

SEQ ID NO: 27 - protein, 1054 aa, Artificial Sequence
  Synthetic
  MISC_FEATURE: Cas12 (NCBI Reference Sequence WP_142383421.1)
MNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIV

SEQ ID NO: 28 - protein, 18 aa, Artificial Sequence
  Synthetic
  MISC_FEATURE: NLS
KRTADGSEFESPKKKRKV

SEQ ID NO: 29 - protein, 791 aa, Artificial Sequence
  Synthetic
  MISC_FEATURE: Cas13b (NCBI Reference Sequence WP_002664492.1)
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQ